(19) United States
(12) Patent Application Publication
(10) Pub. No.: US 2025/0387278 A1
(43) Pub. Date: Dec. 25, 2025

(54) EMERGENCY CASE AND USE OF AN EMERGENCY CASE

(71) Applicant: W. Söhngen GmbH, Taunusstein (DE)

(72) Inventors: Melanie MAY, Wiesbaden (DE); Pascal ARNOLD, Heusweiler (DE)

(21) Appl. No.: 18/750,029

(22) Filed: Jun. 21, 2024

(30) Foreign Application Priority Data
Aug. 28, 2023 (EP) 23193760.8
Oct. 24, 2023 (EP) 23205486.6

(51) Int. Cl. A61F 17/00; A45C 5/00; A45C 13/02; A45C 15/00; C09K 11/77

(52) U.S. Cl. CPC A61F 17/00; A45C 5/00; A45C 13/02; A45C 15/00; C09K 11/7792

(57) ABSTRACT

An emergency case (100, 200) comprises at least one case shell (10), wherein a handle (12) is formed in the case shell (10). Shoulder ends of the handle are located on the upper outer corners (14a, 14b) of the case shell (10). First portions (16a, 16b) of the handle extend vertically upward, and second portions (18a, 18b) extend towards the inner longitudinal edge of the case shell. A third portion connects the two second portions (18a, 18b) along the inner longitudinal edge (22).

### FIELD OF THE INVENTION

The present disclosure relates to workstations at which display monitors and other equipment is made available to one or more operators.

The invention relates to an emergency case.

The invention relates as well to a phosphorescent first aid case, in particular a phosphorescent emergency case or first aid kit. It also relates to a method for producing a phosphorescent first aid case.

In the present case, an emergency case is in particular understood here to be a case that is filled with at least one of: plasters and dressings, disposable gloves, first aid scissors and tweezers, hygienic face masks, hygienic clothing, emergency showers and eyewash kits, disinfectants and dispensers therefor, defibrillators and accessories, and respiratory masks.

In the present case, an emergency case is considered to be covered by the term “first aid case” within the meaning of the invention. In the present case, first aid cases are in particular understood to be those first aid cases that comply with German DIN standards 13157 or 13169.

The invention relates in particular to the configuration of a handle on the emergency case.

### BACKGROUND OF THE INVENTION

An emergency case comprising two handle parts on two halves that open relative to one another is known from CN 0214550771 U.

Furthermore, from emergency medicine, a divisible case according to CN 208877606 U is known in which a handle part is arranged on each of the components, wherein the two handle parts complement one another when the parts of the case are joined together.

The invention relates, inter alia, to the configuration of decoration and/or information provided on the first aid case.

The German utility model DE 86 00 553 U1 discloses a first aid case which comprises two case shells, of which two centrally mounted knob apparatuses are attached to one case shell. The knob apparatuses are configured as a wheel of which one segment is cut out. Two pins which correspond to the knobs and are surrounded when the knobs are rotated are provided on the other half. The single handle for the first aid case is also located on the case half comprising the pins. The two case halves thus have to be produced independently of one another in any case before they are interconnected to form the first aid case.

The European patent application having the file reference EP23152967.8 describes a first aid case that consists of two emergency case halves.

In other prior art, first aid cases are known of which the visibility is increased by affixing highly reflective or fluorescent stickers. In particular, in the prior art, the so-called “first aid cross”, which is a white cross on a green or red background, is affixed to first aid cases in the form of a highly reflective or fluorescent sticker.

EP 0 691 114 A2 discloses an emergency case which comprises two hard shells which can be interconnected in a hinged manner. Inserts are introduced into the hard shells, and partition walls can in turn be introduced into the inserts in order to divide the inserts into compartments.

DE 88 14 319 U1 describes providing partition walls in a container for receiving first aid materials, with notched bars being attached to the inside of the side walls for fastening the partition walls.

### SUMMARY OF THE INVENTION

The problem addressed by the invention is to provide an improved emergency case and to facilitate corresponding uses.

A further problem addressed by the invention is to provide an improved first aid case having increased visibility, and to provide a method by means of which a first aid case of this kind can be produced.

A further problem addressed by the invention is to provide an improved emergency case and to facilitate corresponding uses.

A further problem addressed by the invention is to provide an emergency case comprising an improved seal and a double emergency case comprising improved seals, and to provide a method by means of which an emergency case comprising an improved seal can be produced.

The emergency case according to the invention comprises in one possible embodiment at least one case shell, wherein a handle is formed on the case shell. This handle is arranged with two shoulder ends each on an upper outer corner of the case shell. The term “upper outer corner” relates to an upright position of the emergency case. Proceeding from each of the upper outer corners, the handle extends vertically upward in a first portion. (Since there are two outer corners, there are two first portions.) In a second portion preferably following the first portion, the handle extends toward the inner longitudinal edge of the case shell remote from the (outer) corners. (Since the first portion extends vertically upward, this should be understood to mean that the second portion likewise extends above the case shell upper face, wherein the extension toward the inner longitudinal edge of the case shell can also be formulated such that, in plan view, the second portion reaches the inner longitudinal edge.) A third portion of the handle connecting the two second portions further extends along the inner longitudinal edge. In other words, the handle extends vertically upward from a first upper outer corner in a first portion, then to the inner longitudinal edge in the second portion, then along the inner longitudinal edge in the third portion, then back toward the outer face of the case shell in another second portion, and then vertically downward toward the second outer corner.

An emergency case of this kind has the advantage that, by fastening the handle to the outer corners and owing to the presence of the third portion along the inner longitudinal edge, transport is facilitated, because the handle itself ensures that the case held by hand on the third portion is balanced out.

In addition, use of the emergency case as set out in the claims includes the followings: proceeding from the situation whereby the emergency case is lying flat on an outer face of the case shell or is lying with an outer face of the case shell on the ground or another surface, the user can grasp the third portion of the case with their hand and pull it toward them. This use is not possible in any way with conventional emergency cases that have rounded handles, for example.

Emergency cases have to be able to be carried easily and to be rapidly available. The emergency case according to the invention is easily carried and rapidly available. It can also be readily used when a doctor providing treatment is standing or kneeling beside the patient.

The doctor or other ambulance personnel or the like can in particular set down the case on the ground beside a patient, then kneel down to the patient and pull the case over by stretching their arms. As a result, valuable time can be saved during treatment of the patient.

According to one embodiment of the invention, the transition between the first portion and the second portion is rounded so that there are no disruptive corners. Alternatively or additionally, the transition between the second portion and the third portion is rounded so that there are no disruptive corners.

In one embodiment, the second portion does not extend toward the inner longitudinal edge immediately from the point at which the first portion ends. Instead, it may be advantageous for the second portion of the handle to initially still extend in parallel with an outer longitudinal edge of the case shell in one section (i.e. partially) and to extend toward the inner longitudinal edge in a straight line only in a further section, wherein the section having the straight extension is not necessarily perpendicular to the outer longitudinal edge, but instead can be at a suitable angle of 15 to 30°, for example. As a result, the case not only has a pleasing appearance, but also, functionally, it is easier to grasp from above, and at the same time it is also ensured that the weight is balanced out when holding the case.

The third portion can extend centrally along the inner longitudinal edge. This is of course a particularly stable embodiment when the third portion, which is easily grasped by the user, extends centrally.

In another embodiment, each handle is formed to be symmetrical, i.e. the first portions and the second portions are mirror inverted relative to one another, and the third portion connects them. The mirror plane intersects the third portion into two halves.

In principle, the emergency case can consist of just one single case shell. It can, however, also comprise two case shells that are complementary to one another. This can have the advantage that the case can be configured to be opened up easily, in particular symmetrically.

When a handle is provided on each of the two case shells, with the handles preferably being symmetrical to one another, i.e. configured to be mirrored, the entire emergency case can be carried in a particularly stable manner. This is particularly applicable when the third portions of the handles contact one another.

In another embodiment, a stacking centering apparatus is formed at the end of each handle. In the present case, a “stacking centering apparatus” is understood such that a plurality of identical emergency cases or case shells can comprise stacking centering apparatuses that are complementary to one another, such that they can be stacked in a stable manner by means of an interlocking fit, if not a latching fit.

The stacking centering apparatuses at the end of each handle, i.e. at the outer corners, of course correspond to the stacking centering apparatuses of a second emergency case, again at the outer corners. Stacking centering apparatuses can also be provided at the lower outer corners of the case shell. It may then be unimportant which way the emergency cases (that are being stacked) are oriented.

In another embodiment of the emergency case comprising case shells that are complementary to one another, a handle is formed only on one of the two case shells, wherein a cover cap is provided on each of the outer corners of the case shell without a handle. In this way, a case shell can be provided with either a handle or a cover cap, e.g. by means of a suitable plug-in connection.

A stacking centering apparatus can also be formed at each of the cover caps, and the apparatus is complementary to the stacking centering apparatuses of other cover caps or is complementary to one another at the ends of the handles.

The stacking centering apparatuses of a case shell are preferably complementary to one another at the upper outer corners (so as to be reversed). Identical case shells can then be easily stacked up.

Each case shell is preferably part of an emergency case half, which is detachably connected to another emergency case half. By means of the ability to establish a detachable connection, the emergency case can be separated into two parts.

This is particularly advantageous when both emergency case halves, i.e. both case shells, have a handle.

In another embodiment, each case shell (in the preceding example, each emergency case half) has an inner lid. This makes it possible to carry a case shell on its own, and specifically by the handle, and the specific configuration of the handle having the third portion above the inner longitudinal edge ensures stabilization.

Stacking centering apparatuses can also be provided on each inner lid, such that the case shells can be stacked individually.

According to another embodiment, an emergency case is provided that fluoresces. In this way, it is clearly visible at night, meaning that time can be saved during use.

For example, the emergency case can have walls that are configured to be at least partially fluorescent. This can be achieved when using plastics material for example by a fluorescent substance being added to the plastics granulate before injection molding or another molding process. This can for example be a photoluminescent substance, in particular a photoluminescent substance having a long time of persistence, by means of which a light density of 55/8 mcd/qm in accordance with DIN 67510 can be obtained, for example. For example, the fluorescent substance can be the substance known per se from EP0853112A1; in particular the substance can contain fluorescent particles, in particular with upconversion and/or downconversion. In this case, the particles also glow when there has not been any stimulation by ambient light for a prolonged period of time, for example in the event of a power outage. Residual radiation in a different frequency range is then emitted by upconversion or downconversion of these particles in the visible range such that the emergency case also remains clearly visible in this case.

Alternatively or additionally, the walls of the emergency case can be printed to be fluorescent and/or provided with an adhesive fluorescent film.

In addition, it is possible for the handle to fluoresce. A handle that is configured to be at least partially fluorescent and/or printed to be fluorescent and/or provided with an adhesive fluorescent film can therefore be provided. This can in particular be implemented on a handle insert. The rest of the handle, and potentially also the rest of the emergency case, may not be configured to be fluorescent, which makes production more cost-effective.

This can include the fact that the handle alone glows. The handle is then particularly clearly visible, and so it is easier to grasp in darkness before pulling it over. This effect can likewise be obtained in fluorescent walls, where the handle fluoresces more brightly than the walls. The material for the handle can comprise a greater proportion of fluorescent particles than the rest of the emergency case.

At least one section of the first aid case according to the invention contains a phosphorescent material. Here, the phosphorescent material has been applied to an outer surface of the first aid case in one layer as part of a hot stamping process.

Owing to the phosphorescent material, the first aid case can be particularly easily found even in darkness.

Hot stamping is known per se. During hot stamping, a heated stamping die presses hot stamping foil against the object to be stamped, which is retained on a mount (“retaining frame”). The hot stamping foil is provided in the form of a strip, from which a new strip portion is guided between the stamping die and the object to be stamped in each stamping process. The structuring heated stamping die detaches the transfer sheet of the hot stamping foil from the carrier foil by a separation layer of the transfer sheet adjacent to the carrier foil being melted, which separation layer consists of wax or wax-containing compounds. The transfer sheet is thus applied to the object to be stamped in a structure according to the stamping shape of the stamping die, and it adheres to the object by means of a heat-structurable adhesive layer (which forms the end of the hot stamping foil opposite the stamping foil) of the transfer sheet. A protective coating layer between the separation layer and the adhesive layer generally constitutes the visible element which is intended to be provided during the hot stamping.

CN 113442622 A discloses a hot stamping foil comprising a fluorescent layer. According to the invention, phosphorescent material is intended to be provided here, and in particular phosphorescent particles are intended to be contained in a layer of the transfer sheet, preferably the protective coating layer.

The hot stamping for providing the phosphorescent material has the advantage that, unlike the use of stickers, for example, the application can be carried out using a machine, for example in a factory immediately after the process for injection molding the base for the hot stamping process (for example: the shell for the first aid case). A layer applied by hot stamping also withstands mechanical stresses in an improved manner. In addition, improved hygiene is ensured, which is desirable for medical products.

According to a preferred embodiment, the phosphorescent material is provided in the form of a pigment which contains particles of SrAl₂O₄:Eu, Dy. This pigment is available on the market at a relatively low cost and phosphoresces for quite a long time. The particles preferably have average diameters d₉₀≤5 μm. It has been found that pigments of this size can be processed particularly well for forming hot stamping foils. Any larger pigments that are present can be subjected to a rolling process such that the smaller particle size is obtained. The particles can be provided with a suitable protective coating, preferably assisted by a solvent, for forming the protective coating layer on the transfer sheet of the hot stamping foil.

According to another preferred embodiment of the invention, the layer containing the phosphorescent material has a layer thickness of between 5 and 15 g/m², preferably of between 9 and 13 g/m², particularly preferably of between 10 and 12 g/m². For instance, a layer thickness of approx. 11 g/m² has been found to be particularly advantageous.

As already mentioned, the phosphorescent material is preferably provided in one layer, and in particular in the form of the above-mentioned pigments, which also contains a protective coating and/or a colored coating, for example in order to also make the structure visible in daylight by means of color.

According to another preferred embodiment of the invention, a layer is provided below the phosphorescent material for enhancing the (visibility of the) phosphorescence: this means that a further layer, also preferably applied in the hot stamping process, is provided between the layer containing the phosphorescent material and the outer surface, which is transparent or comprises a white coating.

Furthermore, the layer containing the phosphorescent material preferably provides decoration and/or information, in particular in the form of lettering and/or at least one symbol. As already mentioned, owing to the phosphorescent material, the first aid case can in particular be easily found in darkness. The decoration can be such that a typical user immediately recognizes the first aid case. The information can refer to the function as a first aid case (for example simply by means of lettering reading “First Aid Kit” or the like). The symbol can be in the form of a cross, which is usually known as a red cross. In particular, when also using a red colored coating and/or a white colored coating in the layer containing the phosphorescent material, the decoration and/or information is visible in daylight.

The section containing phosphorescent material is a region of a wall of a case shell or the wall itself or the entire case shell and/or at least one region of an add-on part that can be affixed, in particular of a functional element of the first aid case, such as a handle, or a component of the handle, a closure, or a component of the closure, such as an actuating element of the closure, or stacking centering apparatuses. It is also conceivable for the sections to have an otherwise purely esthetic function aside from the function of phosphorescence, as is the case for insert parts in the handle or decorative strips, for example. It is only the invention that accordingly gives them a significant technical benefit. Sections that contain a phosphorescent material have several advantages. Owing to the persistence, which preferably remains for several hours, once it has been exposed to light the first aid case can also be easily found even in dimly lit conditions or in the dark, and this is advantageous in particular in the event of a power outage or in dimly lit conditions. By only sections of the first aid case containing the phosphorescent material, the production costs are reduced, since, for example, phosphorescent materials are more expensive than thermoplastic materials, of which the components of a first aid case usually consist. This is applicable both when providing the phosphorescent material by means of a hot stamping process and also in a supplementary manner when the phosphorescent material has been added to the thermoplastic material before production. Use for military purposes is also particularly advantageous, since a light source is not required to be able to easily see the first aid case, for example during operations at night.

According to advantageous embodiments of the invention, the first aid case comprises two case shells and the outer surface is provided on an outer wall of one case shell. Alternatively or additionally, the outer surface can be provided on add-on parts that can be affixed to at least one of the case shells.

Alternatively or additionally again, bodies can be add-on parts which can be affixed to the case shells in the form of sections which contain the phosphorescent material, wherein the sections contain the phosphorescent material as part of the material that forms the sections in the form of bodies. In this aspect, in addition to the phosphorescent material applied by hot stamping, further phosphorescent material is provided in the bodies/add-on parts in the material forming the bodies/add-on parts itself.

Usually, the case shells and/or the add-on parts that can be affixed are injection-molded parts made of plastics material, wherein the add-on parts are affixed to the case shells later. For example, the case shells can be produced in a first injection-molding process from a thermoplastic material, such as acrylonitrile butadiene styrene or polypropylene. In a second injection-molding process, the add-on parts that can be affixed are produced from the same thermoplastic material, with phosphorescent material having been added thereto before this injection-molding process. Therefore, the production processes do not need to be modified in a complex manner, since case shells and add-on parts are readily produced by injection molding. In addition, the production costs are thus reduced by the targeted use of the costly phosphorescent material. By affixing the phosphorescent add-on parts later, the flexibility in terms of production is increased, since different sections of the first aid case can be configured to be phosphorescent in this way. The add-on parts are, for example, connected to the case shells by means of a snap-in connector made of plastics material and/or the region of the add-on part that contains the phosphorescent material is connected to the add-on part itself by means of a snap-in connector. The snap-in connector can be dimensioned such that it cannot be manually detached.

According to an advantageous embodiment in this regard, the add-on parts that can be affixed are selected at least from one of the following elements: a handle; a component of the handle, in particular an insert part; a closure, or a component of the closure, in particular an actuating element; a stacking centering apparatus; a decorative strip. The phosphorescent configuration of the above-mentioned add-on parts has the advantage that the functional elements, in particular the handle and the closures, are clearly visible for several hours even in dimly lit conditions or in the dark. The increased visibility makes it possible for the first aider, doctor, or other user providing treatment to find the functional elements more easily and rapidly.

If the handle and/or a case shell according to claim 10, in the form of bodies, contain phosphorescent material, the visibility is increased yet further and the first aid case itself is also visible from a greater distance in dimly lit conditions and can be found accordingly rapidly.

If the phosphorescent material has a time of persistence of at least 1 hour, preferably at least 2 hours, particularly preferably at least 3 hours, the visibility of the accordingly configured sections is increased over a particularly long period of time.

If a body of the first aid case consists of a material that contains phosphorescent material, and the mass percentage of the phosphorescent material in this body is at least 10%, preferably at least 30%, the luminosity is particularly high, which in turn further improves the visibility of the first aid case. If additional phosphorescent material that is not applied by hot stamping is embedded in a body, the pigment can likewise be used which contains particles of SrAl₂O₄:Eu, Dy, which have the now merely preferred average diameters d₅₀=25±4 μm and d₉₀≤60 μm. In this case, the phosphorescent material can be particularly easily and reliably mixed with a thermoplastic material, such as acrylonitrile butadiene styrene or polypropylene. This makes it easier to produce the starting material for molding the phosphorescent add-on parts that can be affixed or the case shells. In addition, owing to the good miscibility and stability against separation, the molding of the phosphorescent add-on parts that can be affixed or the case shells themselves is simplified and the homogeneity of the ready-molded parts is increased.

If the first aid case is filled with at least one of plasters and dressings, disposable gloves, first aid scissors and tweezers, hygienic face masks, hygienic clothing, emergency showers and eyewash kits, disinfectants and dispensers therefor, defibrillators and accessories, or respiratory masks, they can be stored so as to be easy to find and simultaneously protected against contamination.

If at least one surface section of the first aid case is metal-coated, the visibility is further improved by the reflectivity, which is increased in comparison with the non-metal-coated surface, since the light emitted by the phosphorescent material is reflected to a greater extent than would be the case with a standard plastics surface.

The method according to the invention for producing a first aid case includes providing two case shells and a handle, performing at least one hot stamping process, wherein, in the entirety of all the hot stamping processes, a layer containing phosphorescent particles is applied to at least one of the case shells and/or the handle (for example: two hot stamping processes, one for a case shell and one for the handle; or two hot stamping processes, one for each of the two case shells and none for the handle; or three hot stamping processes, for both case shells and the handle). After connecting the two case shells to form a first aid case, the handle is affixed to a case shell. Two handles can also be provided, wherein one handle is preferably affixed to another case shell in each case.

The method according to the invention results in the production of a first aid case having the advantage of it being easy to find in darkness and the above-mentioned advantages of providing the phosphorescent material in a hot stamping layer, as mentioned above.

According to a preferred embodiment in this regard, each hot stamping process is performed using a hot stamping die which is brought to a temperature of between 130° C. and 165° C. This temperature has been found to be advantageous for applying the phosphorescent material particularly effectively. This is applicable in particular for application to a body made of a thermoplastic material, such as acrylonitrile butadiene styrene or polypropylene, which is the preferred material for a case shell, for example. This material does not melt at 130° C. to 165° C.

The hot stamping die is preferably pressed against the case shell or the handle for a duration of between 0.25 seconds and 5 seconds and/or preferably at a pressure of between 3 and 9 bar, preferably between 5 and 7 bar (for example: at 6 bar) and/or preferably by applying a weight of between 600 and 1000 kg, preferably between 720 and 880 kg (for example: of 800 kg). It has been found that these parameter values particularly reliably ensure that the layer containing phosphorescent material applied by hot stamping is retained for a long time.

According to a preferred embodiment of the method, the case shells and preferably also the handle are produced from a thermoplastic material, preferably from an acrylonitrile butadiene styrene plastics material, in a first injection-molding process. The phosphorescent material is then applied to an outer wall of at least one case shell and/or the handle by hot stamping.

It is also possible that, in a second injection-molding process, add-on parts that can be affixed to at least one case shell are produced from a thermoplastic material, preferably from an acrylonitrile butadiene styrene plastics material, with the phosphorescent material being added to the plastics material before the injection-molding process. In this way, the already existing infrastructure, in particular the costly injection molds, can be used. Production in two separate injection-molding processes also makes it easier to use the phosphorescent materials, which have a high cost in comparison with thermoplastic materials, in a targeted and reduced manner.

For particularly good visibility, phosphorescent material can also be added to the thermoplastic material for producing the case shells and/or the handle even before the first injection-molding process. As a result, all the parts of the first aid case can be molded from the same starting material, which simplifies production. Preferably, in each hot stamping process, a hot stamping foil is used, which comprises a transfer sheet that contains phosphorescent particles of SrAl₂O₄:Eu, Dy. The fact that these particles are advantageous has already been set out above. This is preferably and particularly applicable when the phosphorescent particles have average diameters d₉₀≤5 μm.

In addition, the visibility of a first aid case produced using this method is particularly high, since additional regions facing the first aider, doctor, or other user phosphoresce.

The innovative hot stamping foil comprises:
a carrier foil and
a transfer sheet,
   wherein the transfer sheet comprises:
a separation layer, a protective coating layer, and a heat-activated adhesive layer, and
   according to the invention the protective coating layer also comprises phosphorescent particles in addition to the protective coating.

Advantageously, the layer is transparent below the phosphorescent layer or consists of a white coating, such that, in the innovative hot stamping foil, a transparent layer or a layer of white coating is arranged between the protective coating layer containing the phosphorescent particles and the adhesive layer.

If at least surface sections of the case shells and/or the handle are additionally metal-coated, the visibility of the thus produced first aid case is yet higher owing to the increased reflectivity of the metal-coated surface sections.

According to an advantageous embodiment in this regard, the metal-coating is implemented by means of the following steps:
etching the surface sections of the first aid case with a solution consisting of chromic acid, sulfuric acid, and water, wherein the etching is carried out at a temperature of at least 60° C., preferably at 70° C.;
activating the surface sections with palladium, wherein the palladium is colloidally dissolved and is surrounded by a protective colloid shell or is present in ionic form;
wetting the surface sections with an accelerator solution, wherein the accelerator solution is selected such that it reduces ionically present palladium or, in the case of colloidally dissolved palladium, removes the protective colloid shell surrounding the colloidal palladium;
chemically depositing nickel on the surface sections;
galvanically depositing copper on the nickel-plated surface sections;
galvanically depositing nickel on the copper-plated surface sections;
galvanically depositing chromium on the surface sections nickel-plated by galvanic deposition;
drying the surface sections, preferably at a temperature of 67° C.,
wherein the surface sections are rinsed with water, preferably with deionized water, between the individual steps.

In this way, by means of a layered structure, a particularly homogeneous chromium layer is generated as the outermost layer of the metal-coated region. This chromium layer has particularly high reflectivity, which increases the visibility of the phosphorescent sections yet further.

Moreover, the features and advantages relating to the first aid case are accordingly transferable to the method, and vice versa, and therefore no further repetitions are necessary at this point.

The emergency case according to the invention thus comprises in a possible embodiment a first case shell and a second case shell, and a first knob apparatus on the first case shell, which can be brought from a rest position into a closed position and vice versa, and further comprises a second knob apparatus on the second case shell, which likewise can be brought from a rest position into a closed position and vice versa, wherein, when both knob apparatuses are in a given rest position, the emergency case is open and wherein, in the closed position of the first knob apparatus, the first knob apparatus engages behind a body formed on the second case shell by means of a rear-engaging element, and wherein, in the closed position of the second knob apparatus, the second knob apparatus engages behind a body formed on the first case shell by means of a rear-engaging element.

The invention thus proceeds from the fact that the knob apparatuses are both provided on one case shell. Therefore, the stability of the assembly is increased. The emergency case thus has a particularly solid construction.

According to embodiments of the invention, it can be provided that the two case shells are detachable from one another. This embodiment can be advantageous, inter alia when only one case shell is required, or when two first aiders working together on one patient have an emergency case of the above-mentioned type and each want to attend to them from one of the case shells using the material found therein.

Particularly preferably, it is provided here that the emergency case comprises two emergency case halves, which each comprise a case shell and an inner lid, wherein the emergency case halves are detachably connected, and wherein each inner lid can be locked to its case shell to produce a closed emergency case half.

By providing separable case shells and by providing the inner lid, the case shells can be transported on their own, i.e. can also be used on their own as an emergency case, and because of the lockable inner lid the material (plasters and dressings, disposable gloves, etc., as stated above) found in the respective emergency case half does not fall out in the process. The emergency case can thus be used flexibly:

the two emergency case halves can remain connected; the first aider, doctor, or other user providing treatment then has a large emergency case containing a lot of material. When the emergency case halves are equipped identically, a particularly large amount of dressings are then provided, for example. When the emergency case halves are equipped differently, the contents can complement one another. When the emergency case halves are separated, they can each be used by two different users. Where necessary, they can be separated rapidly on site. When an emergency case is provided, two users can thus each use one emergency case half and can accordingly treat two patients simultaneously.

According to a preferred embodiment, the emergency case halves are detachably connected solely by means located on the case shells. In this case, in particular auxiliary means such as screws and the like can be omitted. In this embodiment, it is typically ensured that the emergency case halves can be detached from one another particularly rapidly, such that the above-described separation of the emergency case for two users is possible on site at short notice.

According to another preferred embodiment, the emergency case comprises two identically constructed case shells. This can not only provide the advantage of symmetry, but also the connection means potentially located on the case shell can be configured to be complementary to one another. To produce an emergency case, two different case shells do not then need to be provided.

In this context, it is preferably provided that a rod and, coaxially therewith and so as to be reversed, a connection device for the rod of the other case shell are arranged on each case shell. The rod then just needs to be introduced into (pressed into or plugged into) the connection device that is preferably C-shaped in cross section in order to interconnect the emergency case halves, or, vice versa, the rods can be detached again when the connection device has sufficient elasticity. If this is done in both connection devices for the respective rods, the emergency case can be separated into the two emergency case halves.

More preferably, it is provided here that coaxially extending hinges for the respective inner lid of the case shell are arranged between the rod and the connection device, wherein the hinges of one case shell engage in gaps between the hinges for the other case shell. This can in particular be configured such that the gaps are completely closed except for a small clearance (where necessary, latching can even be provided);

therefore, a practically continuous axis is provided both for the case shells and for the inner lid, which makes it possible to rotate the case shell halves relative to one another and to rotate the inner lid separately.

According to a preferred embodiment of the present invention, each knob apparatus is rotatable. A rear-engaging element engaging behind a body can be implemented in a particularly simple manner when the rear-engaging element is rotated behind the body, i.e. the entire knob apparatus is rotatable.

In a preferred embodiment, it is provided here that each knob apparatus is arranged on the front left when the associated case shell is opened upward (i.e. on the front right when the associated case shell is opened downward) and can be rotated counterclockwise from the rest position into the closed position. When the knob apparatus is arranged on the left and can then be rotated counterclockwise, this corresponds to an outward movement, which can be easily performed from the wrist or elbow. The closure of the second knob apparatus, which is to be performed at the same time, from the front right counterclockwise downward can be performed practically synchronously from the right wrist downward. This closing process is particularly intuitive. The reverse opening process (right hand turns upward, the left downward) is also helpful.

According to another advantageous embodiment of the invention, each knob apparatus can comprise a knob, for which a geometric midpoint can be defined. An axis of rotation of the knob apparatus is then eccentric to the geometric midpoint of the knob.

In this aspect, the knob can be pivoted in a much improved manner and is thus easier to operate. This aspect can in turn constitute a separate invention, independently of the above-mentioned invention: An emergency case comprising a first case shell and a second case shell and comprising a knob apparatus on each case shell, wherein each knob apparatus comprises a knob, for which a geometric midpoint can be defined, and wherein an axis of rotation of the knob apparatus is eccentric to the geometric midpoint of the knob.

According to preferred embodiments of the invention mentioned at the outset and of further aspects of the invention as mentioned below, each knob apparatus comprises a cross-shaped knob. Unlike wheels, the cross shape is advantageous here because, on the basis of the cross shape, in particular also in connection with the eccentric mounting of the knob apparatus, it is easier to see what position the knob apparatus is in.

It is preferably provided here that legs of equal length of the cross shape are at an angle of 90° to the respectively adjacent leg. (The cross therefore has exactly four identical legs.) This makes it easier to operate.

According to embodiments, a (rotary) shaft of the knob apparatus can be formed on a first leg of the cross shape, as a result of which the above-mentioned embodiment of the eccentric mounting can be implemented.

The cross is therefore not rotated about the midpoint from which the four legs protrude, i.e. not about the geometric midpoint, but instead about one of the legs, such that the pivot range of the knob apparatus is enlarged, which in turn makes it easier to operate. This is particularly applicable in connection with the embodiment of the rotatability for closure in the clockwise direction for the knob apparatus arranged on the front left (relative to the open case shell).

Advantageously, it can be provided here that the rear-engaging element is formed on the leg of the cross shape opposite the first leg. The advantage of the cross shape is also clear here: the cross shape can be configured to be particularly stable owing to side walls on the legs, and this likewise makes it easier to operate.

According to embodiments of the invention in all the described aspects of the invention, including additional concepts that are independent in nature and the like, the body behind which a knob apparatus engages or can engage can be formed as a lug. For this purpose, it preferably comprises a latching mechanism. Unlike a simple pin, the lug has the advantage of stability of the closure, since the two case shells are thus hooked into one another in a certain way. The hold is potentially further enhanced by the latching mechanism.

According to advantageous embodiments of the invention, there is also a stowed position in addition to the rest position and the closed position. This can be described such that each knob apparatus can be rotated from the rest position in a first rotational direction, preferably by 90°, to produce the closed position, and wherein each knob apparatus can be rotated from the rest position in a second rotational direction counter to the first rotational direction, preferably by 90°, to produce a stowed position.

This can have the advantage that the rear-engaging element causes less disruption, in order to make it easier to remove objects from the emergency case. This embodiment is particularly advantageous in connection with the eccentric mounting of the knob apparatus, and more particularly also in connection with the cross shape.

When the cross is pivoted, it can cause particularly low disruption in the stowed position.

According to another preferred embodiment, at least one of the emergency case halves comprises a handle on the case shell. The emergency case can be particularly easily carried by means of a handle.

In one embodiment, both emergency case halves comprise a handle; the two emergency case halves can each be carried on their own after being detached from one another.

Alternatively, just one emergency case half comprises a handle. This can be useful if particularly important material is stored in the respective case shell and if material that is not always used is stored in the other case shell. The emergency case half containing the more important material can then be separated and the other emergency case half can be used only when needed. In this case, it is preferably provided on the case shell without a handle that cover caps are provided at the point at which the handle would otherwise engage or be attached.

Preferably, each handle is arranged with two shoulder ends each on an upper outer corner of the case shell. The term “upper outer corner” relates to an upright position of the emergency case. Proceeding from each of the upper outer corners, the handle extends vertically upward in a first portion. (Since there are two outer corners, there are two first portions.) In a second portion preferably following the first portion, the handle extends toward the inner longitudinal edge of the case shell remote from the (outer) corners. (Since the first portion extends vertically upward, this should be understood to mean that the second portion likewise extends above the case shell upper face, wherein the extension toward the inner longitudinal edge of the case shell can also be formulated such that, in plan view, the second portion reaches the inner longitudinal edge.) A third portion of the handle connecting the two second portions further extends along the inner longitudinal edge. In other words, the handle extends vertically upward proceeding from a first upper outer corner in a first portion, then to the inner longitudinal edge in the second portion, then along the inner longitudinal edge in the third portion, then back toward the outer face of the case shell in the second portion, and then vertically downward toward the second outer corner.

An emergency case of this kind has the advantage that, by fastening the handle to the outer corners and owing to the presence of the third portion along the inner longitudinal edge, first of all, transport is facilitated, because the handle itself ensures that the case held by hand on the third portion is balanced out. In addition, the use makes it possible that, proceeding from the situation whereby the emergency case is lying flat on an outer face of the case shell or is lying with an outer face of the case shell on the ground or another surface, the user can grasp the third portion of the case with their hand and pull it toward them. This use is not possible in any way with conventional emergency cases that have rounded handles, for example.

Emergency cases have to be able to be carried easily and to be rapidly available. This also applies to these emergency cases according to the preferred embodiment: it can also be readily used when a first aider providing treatment is standing or kneeling beside the patient.

The doctor or other ambulance personnel or the like, as the first aider, can in particular set down the case on the ground beside a patient, then kneel down to the patient and pull the case over by stretching their arms. As a result, valuable time can be saved during treatment of the patient.

According to a preferred embodiment, in this aspect, the transition between the first portion and the second portion is rounded so that there are no disruptive corners. Alternatively or additionally, the transition between the second portion and the third portion is rounded so that there are no disruptive corners.

The second portion does not have to extend toward the inner longitudinal edge immediately from the point at which the first portion ends. Instead, it may be advantageous for the second portion of the handle to initially still extend in parallel with an outer longitudinal edge of the case shell in one section (i.e. partially) and to extend toward the inner longitudinal edge in a straight line only in a further section, wherein the section having the straight extension is not necessarily perpendicular to the outer longitudinal edge, but instead can be at a suitable angle of 15 to 30°, for example. As a result, the case not only has a pleasing appearance, but also, functionally, it is easier to grasp from above, and at the same time it is also ensured that the weight is balanced out when holding the case.

The third portion can extend centrally along the inner longitudinal edge. This is of course a particularly stable embodiment when the third portion, which is easily grasped by the user, extends centrally.

More preferably, each handle is then also formed to be symmetrical, i.e. the first portions and the second portions are mirror inverted relative to one another, and the third portion connects them. The mirror plane intersects the third portion into two halves.

When a handle is provided on each of the two case shells, with the handles preferably being symmetrical to one another, i.e. configured to be mirrored, the entire emergency case can be carried in a particularly stable manner. This is particularly applicable when the third portions of the handles contact one another.

It is preferably also provided that a stacking centering apparatus is formed at the end of each handle.

According to another preferred embodiment of the invention, stacking centering apparatuses are formed on the case shell and/or on the inner lid, with these preferably being arranged so as to be reversed.

In the present case, a “stacking centering apparatus” is understood such that a plurality of identical emergency cases or case shells can comprise stacking centering apparatuses that are complementary to one another, such that they can be stacked in a stable manner by means of an interlocking fit, if not even a latching fit. It is preferably provided here that a stacking centering apparatus is formed at the end of each handle.

The stacking centering apparatuses at the end of each handle, i.e. at the outer corners, of course correspond to the stacking centering apparatuses of a second emergency case, again at the outer corners. Stacking centering apparatuses can also be provided at the lower outer corners of the case shell. It may then be unimportant which way around the emergency cases that are being stacked are oriented.

A stacking centering apparatus can also be formed at each of the cover caps, which apparatus is then complementary to the stacking centering apparatuses of other cover caps or are complementary to one another at the ends of the handles.

The stacking centering apparatuses of a case shell are preferably complementary to one another at the upper outer corners (so as to be reversed). Identical case shells can then be easily stacked up.

Stacking centering apparatuses can also be provided on each inner lid, such that the case shells can be stacked individually.

According to a preferred embodiment of the invention, the inner lid is or can be latched to the associated case shell. This ensures increased protection for the material found in the case shell: when the inner lid is latched, it is particularly unlikely that it can fall out.

Furthermore, according to a preferred embodiment, it is provided that each inner lid can be opened by 90° (when the case is suitably at least partially open). Therefore, each emergency case half can be opened up to an extent, and the doctor or other user providing treatment as the first aider can easily remove the material found therein.

Alternatively or additionally, it is possible that, when the case is open, an inner lid (and preferably either of the two inner lids) is rotatable by 180°, such that it comes to rest on the other inner lid. In this way, the inner lid does not cause any disruption and material can be easily removed from this one emergency case half.

The two features of opening by 90° and rotating by 180° can both be implemented as desired, such that material can either be removed from both emergency case halves or the inner lid does not cause any disruption and material can be removed only from one emergency case half.

In these two embodiments of opening by 90° and rotating by 180°, it is preferably provided that the inner lids are latched to one another. These lids can in particular be latched to one another using the stacking centering apparatuses which are preferably provided anyway.

The emergency case half according to the invention comprises a case shell and a knob apparatus as well as a body which can be engaged behind, wherein the emergency case half can be connected to an identical case shell which comprises a knob apparatus and a body which can be engaged behind in order to provide an emergency case (in particular of the above-described type in at least one of the above-described embodiments, optionally also in one of the above-described preferred embodiments) in which each knob apparatus of one case shell can engage behind the body which can be engaged behind on the other case shell.

According to embodiments, the emergency case half comprises an inner lid. The case shell can be connected to an identically constructed case shell, in particular can be connected without tools. Said emergency case half can be transported on its own, for example by two users. If the emergency case is no longer required, they can be combined to form one emergency case.

Advantageous embodiments of the above-described emergency case according to the invention of course also apply to the emergency case halves, where applicable.

In particular, the ability to establish a connection to said rods and to the connection device that is complementary thereto without tools can be accomplished on the emergency case half.

A method for connecting two emergency case halves includes the fact that the two rods of the emergency case halves are inserted into the connection device of the respectively other emergency case half.

The use of an emergency case half is the use thereof on its own as an emergency case that is separately portable.

According to another preferred embodiment, it can be provided that the emergency case fluoresces. In this way, it is clearly visible at night, meaning that time can be saved during use.

For example, the emergency case can have walls that are configured to be at least partially fluorescent. This can be achieved when using plastics material for example by a fluorescent substance being added to the plastics granulate before injection molding or another molding process. This can for example be a photoluminescent substance, in particular a photoluminescent substance having a long time of persistence, by means of which a light density of 55/8 mcd/qm in accordance with DIN 67510 can be obtained, for example. For example, the fluorescent substance can be the substance known per se from EP 0 853 112 A1; in particular the substance can contain fluorescent particles, in particular with upconversion and/or downconversion. In this case, the particles also glow when there has not been any stimulation by ambient light for a prolonged period of time, for example in the event of a power outage. Residual radiation in a different frequency range is then emitted by upconversion or downconversion of these particles in the visible range such that the emergency case also remains clearly visible in this case.

Alternatively or additionally, the walls of the emergency case can be printed to be fluorescent and/or provided with an adhesive fluorescent film.

In addition, it is possible for the handle to fluoresce. A handle that is configured to be at least partially fluorescent and/or printed to be fluorescent and/or provided with an adhesive fluorescent film can therefore be provided. This can in particular be implemented on a handle insert. The rest of the handle, and potentially also the rest of the emergency case, then does not have to be configured to be fluorescent, which makes production more cost-effective.

This can include the fact that the handle alone glows. The handle is then particularly clearly visible, and so it is easier to grasp in darkness before pulling it over. This effect can likewise be obtained in the fluorescent walls by the handle fluorescing more brightly than the walls. The material for the handle can comprise a greater proportion of fluorescent particles than the rest of the emergency case.

The emergency case according to the invention thus comprises in one possible embodiment two emergency case halves, which each comprise a case shell. Partition elements for forming compartments are provided in at least one case shell, wherein a series of connection apparatuses is provided (in each case) directly as part of at least two inner walls of this case shell and wherein the partition elements are inserted at least in one part of the connection apparatuses.

By providing the connection apparatuses directly as part of the inner walls (i.e. directly on the inner walls), i.e. by no longer providing the connection apparatuses on other elements inserted into the case shell, as in the case of the notched bars from DE 88 14 319 U1 or the inserts from EP 0 691 114 A2, the connection apparatuses can be provided directly when producing the case shells. This means that production steps (positioning and fastening the notched bars/inserting the inserts) can be avoided. The assembly can also be produced in a stable manner with less effort than in the prior art.

According to embodiments of the invention, the emergency case comprises rectangular case shells. In this way, partition elements, which extend in a straight line, are each inserted in opposite connection apparatuses of a case shell.

Rectangular case shells are commonplace and can be easily stowed away, e.g. can be more easily stacked than other shapes. The fact that the partition elements are straight has the advantage that they can be handled more easily.

According to advantageous embodiments, a reinforcement rib on the outer wall corresponds in position to at least one connection apparatus as part of the inner wall of the case shell, and preferably a reinforcement rib on the outer wall corresponds in position to each connection apparatus on the inner wall of the case shell. Therefore, the connection apparatus can be provided without the case shell losing any stability.

In this embodiment and/or other embodiments, it is advantageous for each case shell to be produced in one piece from hard plastics material, e.g. by injection molding. The same applies to the partition elements, which can each be produced in one piece from hard plastics material. This means that the partition elements are particularly stable. The material for the partition elements can be different from that for the case shell, for example the partition elements can be transparent or partially transparent so as to give a better overview of the contents of the individual compartments.

According to advantageous embodiments, the partition elements comprise a strip-shaped main body, wherein a series of connection apparatuses for other partition elements is provided at least on one side of the strip-shaped main body. This means that small compartments can be provided.

In this context, it is advantageous for the connection apparatuses of the partition elements to each comprise a plurality of lugs, which are arranged to alternate with one another to the right and the left. Even this simple embodiment can be produced with little effort and can be configured to be stable.

According to advantageous embodiments, the partition elements comprise a sliding track, which can be inserted into a connection apparatus, on at least one longitudinal end. It is particularly advantageous for the sliding track to be able to be inserted both into connection apparatuses on the inner walls of the case shells and into connection apparatuses of other partition elements; the partition elements can be used universally.

In the emergency case according to the invention, according to embodiments of the invention, partition elements for forming compartments are provided in each case shell, and the above statements regarding the case shells, their production from hard plastics material, and the partition elements equally apply to both case shells and/or the partition elements located therein.

The emergency case half according to the invention comprises a case shell and a series of (respective) connection apparatuses arranged directly as part of at least two inner walls of the case shell, wherein partition elements for forming compartments are inserted in at least one part of the connection apparatuses. The advantages and advantageous embodiments described with regard to the emergency case equally apply to the emergency case half.

Advantageously, in this context it is provided that partition elements for forming compartments are inserted in one part of the connection apparatuses, but not in the other part. The partition elements can be rearranged in this way where necessary. The series of connection apparatuses each on an inner wall, preferably a series of connection apparatuses per inner wall, allows for flexible use.

According to embodiments, the emergency case half comprises an inner lid, wherein the inner lid can be locked to the case shell to produce a closed emergency case half.

Owing to the inner lid, the emergency case half can also transported on its own.

Because of the lockable inner lid the material found in the respective emergency case half does not fall out in the process.

In connection with the emergency case, it is particularly preferably provided that the emergency case comprises two emergency case halves, which each comprise a case shell and an inner lid, wherein the emergency case halves are detachably connected, and wherein each inner lid can be locked to its case shell to produce a closed emergency case half. This embodiment can be advantageous, inter alia when only one case shell is required, or when two first aiders working together on one patient have an emergency case of the above-mentioned type and each want to attend to them from one of the case shells using the material found therein.

The emergency case can thus be used flexibly: the two emergency case halves can remain connected; the first aider, doctor, or other user providing treatment then has a large emergency case containing a lot of material. When the emergency case halves are equipped identically, a particularly large amount of dressings are then provided, for example. When the emergency case halves are equipped differently, the contents can complement one another. When the emergency case halves are separated, they can each be used by two different users. Where necessary, they can be separated rapidly on site. When an emergency case is provided, two users can thus each use one emergency case half and can accordingly treat two patients simultaneously.

According to a preferred embodiment, the emergency case halves are detachably connected solely by means located on the case shells. In this case, in particular auxiliary means such as screws and the like can be omitted. In this embodiment, it is typically ensured that the emergency case halves can be detached from one another particularly rapidly, such that the above-described separation of the emergency case for two users is possible on site at short notice.

According to another preferred embodiment, the emergency case comprises two identically constructed case shells. This can not only provide the advantage of symmetry, but also the connection means potentially located on the case shell can be configured to be complementary to one another. To produce an emergency case, two different case shells do not then need to be provided.

In this context, it is preferably provided that a rod and, coaxially therewith and so as to be reversed, a connection device for the rod of the other case shell are arranged on each case shell. The rod then just needs to be introduced into (pressed into or plugged into) the connection device that is preferably C-shaped in cross section in order to interconnect the emergency case halves, or, vice versa, the rods can be detached again when the connection device has sufficient elasticity. If this is done in both connection devices for the respective rods, the emergency case can be separated into the two emergency case halves.

More preferably, it is provided here that coaxially extending hinges for the respective inner lid of the case shell are arranged between the rod and the connection device, wherein the hinges of one case shell engage in gaps between the hinges for the other case shell. This can in particular be configured such that the gaps are completely closed except for a small clearance (where necessary, latching can even be provided); therefore, a practically continuous axis is provided both for the case shells and for the inner lid, which makes it possible to rotate the case shell halves relative to one another and to rotate the inner lid separately.

The system according to the invention of emergency cases having the features according to the embodiments described and/or of emergency case halves having the features according to the embodiments described, wherein the emergency case or emergency case halves are of different sizes, has the property whereby the dimensions of the case shells are coordinated with the connection apparatuses among themselves and with the dimensions of the partition elements such that predetermined partition elements can be used in a plurality of emergency cases and/or emergency case halves having case shells of different sizes.

Someone producing the system according to the invention therefore only has to provide the partition elements once in one, two, three, four, five, or, where necessary, more sizes, but can preferably insert all of these partition elements in a plurality of emergency cases/emergency case halves (or in an emergency case and an emergency case half of different sizes). This modular design makes it easier to produce emergency cases or emergency case halves.

This can be possible in an advantageous manner when the partition elements are the same height consistently and when the case shells are also all the same height or are at least approximately the same height with different length-width ratios, so that partition elements of the same height can be used in all the different emergency cases or emergency case halves.

According to advantageous embodiments of the system according to the invention, it is provided that the predetermined partition elements form a group as a kit which can be used in all the emergency cases or emergency case halves of the system. As just mentioned, this is advantageously associated with the use of partition elements of the same height.

The partition element according to the invention for a case shell of an emergency case comprises a strip-shaped main body, on which connection apparatuses are formed, wherein each connection apparatus comprises lugs, which are arranged to alternate with one another to the right and the left.

The advantages of this partition element have already been described above with reference to the emergency case.

According to advantageous embodiments in this regard, connection apparatuses are provided on each side of the strip-shaped main body. A cut-out on one side is opposite each lug on this one side, wherein a lug on the other side corresponds to this opposite cut-out. The lug on one side again corresponds to the corresponding cut-out on the other side. The corresponding cut-out on the other side is therefore accordingly also opposite the lug on the other side. In this embodiment, “hollow lugs” are therefore simply provided, wherein the interior of the lug corresponds to the cut-out on the other side. When viewed from above, the lugs on one side of the partition elements and the lugs on the other side thereof form a kind of zig-zag pattern. Because the lugs are arranged to alternate with one another to the right and the left, the lugs on the other side are also arranged to alternate with one another to the right and the left, since the lug on the right corresponds to each cut-out on the left on the other side. As already stated, this embodiment can be provided by injection molding from hard plastics material, in particular transparent or semi-transparent plastics material.

According to embodiments of the invention, in one aspect, the emergency case comprises a first case shell and a second case shell, and a first knob apparatus on the first case shell, which can be brought from a rest position into a closed position and vice versa, and further comprises a second knob apparatus on the second case shell, which likewise can be brought from a rest position into a closed position and vice versa, wherein, when both knob apparatuses are in a given rest position, the emergency case is open and wherein, in the closed position of the first knob apparatus, the first knob apparatus engages behind a body formed on the second case shell by means of a rear-engaging element, and wherein, in the closed position of the second knob apparatus, the second knob apparatus engages behind a body formed on the first case shell by means of a rear-engaging element.

This embodiment thus proceeds from the fact that the knob apparatuses are both provided on one case shell. Therefore, the stability of the assembly is increased. The emergency case thus has a particularly solid construction.

According to a preferred embodiment of this aspect, each knob apparatus is rotatable. A rear-engaging element engaging behind a body can be implemented in a particularly simple manner when the rear-engaging element is rotated behind the body, i.e. the entire knob apparatus is rotatable.

In a preferred embodiment, it is furthermore provided here that each knob apparatus is arranged on the front left when the associated case shell is opened upward (i.e. on the front right when the associated case shell is opened downward) and can be rotated counterclockwise from the rest position into the closed position. When the knob apparatus is arranged on the left and can then be rotated counterclockwise, this corresponds to an outward movement, which can be easily performed from the wrist or elbow. The closure of the second knob apparatus, which is to be performed at the same time, from the front right counterclockwise downward can be performed practically synchronously from the right wrist downward. This closing process is particularly intuitive. The reverse opening process (right hand turns upward, the left downward) is also helpful.

According to another advantageous embodiment of the aspect comprising the knob apparatus, each knob apparatus can comprise a knob, for which a geometric midpoint can be defined. An axis of rotation of the knob apparatus is then eccentric to the geometric midpoint of the knob.

In this aspect, the knob can be pivoted in a much improved manner and is thus easier to operate. Irrespective of the other aspect comprising the knob apparatus, this aspect can in turn constitute a separate aspect of the invention: An emergency case comprising a first case shell and a second case shell and comprising a knob apparatus on each case shell, wherein each knob apparatus comprises a knob, for which a geometric midpoint can be defined, and wherein an axis of rotation of the knob apparatus is eccentric to the geometric midpoint of the knob.

According to preferred embodiments of all of the above-mentioned aspects of the invention as described above and of further aspects of the invention as mentioned below, each knob apparatus comprises a cross-shaped knob. Unlike wheels, the cross shape is advantageous here because, on the basis of the cross shape, in particular also in connection with the eccentric mounting of the knob apparatus, it is easier to see what position the knob apparatus is in.

It is preferably provided here that legs of equal length of the cross shape are at an angle of 90° to the respectively adjacent leg. (The cross therefore has exactly four identical legs.) This makes it easier to operate.

According to embodiments, a (rotary) shaft of the knob apparatus can be formed on a first leg of the cross shape, as a result of which the above-mentioned embodiment of the eccentric mounting can be implemented.

The cross is therefore not rotated about the midpoint from which the four legs protrude, i.e. not about the geometric midpoint, but instead about one of the legs, such that the pivot range of the knob apparatus is enlarged, which in turn makes it easier to operate. This is particularly applicable in connection with the embodiment of the rotatability for closure in the clockwise direction for the knob apparatus arranged on the front left (relative to the open case shell).

Advantageously, it can be provided here that the rear-engaging element is formed on the leg of the cross shape opposite the first leg. The advantage of the cross shape is also clear here: the cross shape can be configured to be particularly stable owing to side walls on the legs, and this likewise makes it easier to operate.

According to embodiments in all the described aspects comprising the knob apparatus, the body behind which a knob apparatus engages or can engage can be formed as a lug. For this purpose, it preferably comprises a latching mechanism. Unlike a simple pin, the lug has the advantage of stability of the closure, since the two case shells are thus hooked into one another in a certain way. The hold is potentially further enhanced by the latching mechanism.

According to advantageous embodiments of the aspect comprising the knob apparatus, there is also a stowed position in addition to the rest position and the closed position. This can be described such that each knob apparatus can be rotated from the rest position in a first rotational direction, preferably by 90°, to produce the closed position, and wherein each knob apparatus can be rotated from the rest position in a second rotational direction counter to the first rotational direction, preferably by 90°, to produce a stowed position.

This can have the advantage that the rear-engaging element causes less disruption, in order to make it easier to remove objects from the emergency case. This embodiment is particularly advantageous in connection with the eccentric mounting of the knob apparatus, and more particularly also in connection with the cross shape. When the cross is pivoted, it can cause particularly low disruption in the stowed position.

According to another preferred embodiment, at least one of the emergency case halves comprises a handle on the case shell. The emergency case can be particularly easily carried by means of a handle.

In this regard, both emergency case halves comprise a handle; the two emergency case halves can each be carried on their own after being detached from one another.

Alternatively, just one emergency case half comprises a handle. This can be useful if particularly important material is stored in the respective case shell and if material that is not always used is stored in the other case shell. The emergency case half containing the more important material can then be separated and the other emergency case half can be used only when needed. In this case, it is preferably provided on the case shell without a handle that cover caps are provided at the point at which the handle would otherwise engage or be attached.

Preferably, each handle is arranged with two shoulder ends each on an upper outer corner of the case shell. The term “upper outer corner” relates to an upright position of the emergency case. Proceeding from each of the upper outer corners, the handle extends vertically upward in a first portion. (Since there are two outer corners, there are two first portions.) In a second portion preferably following the first portion, the handle extends toward the inner longitudinal edge of the case shell remote from the (outer) corners. (Since the first portion extends vertically upward, this should be understood to mean that the second portion likewise extends above the case shell upper face, wherein the extension toward the inner longitudinal edge of the case shell can also be formulated such that, in plan view, the second portion reaches the inner longitudinal edge.) A third portion of the handle connecting the two second portions further extends along the inner longitudinal edge. In other words, the handle extends vertically upward proceeding from a first upper outer corner in a first portion, then to the inner longitudinal edge in the second portion, then along the inner longitudinal edge in the third portion, then back toward the outer face of the case shell in the second portion, and then vertically downward toward the second outer corner.

An emergency case of this kind has the advantage that, by fastening the handle to the outer corners and owing to the presence of the third portion along the inner longitudinal edge, first of all, transport is facilitated, because the handle itself ensures that the case held by hand on the third portion is balanced out. In addition, the use makes it possible that, proceeding from the situation whereby the emergency case is lying flat on an outer face of the case shell or is lying with an outer face of the case shell on the ground or another surface, the user can grasp the third portion of the case with their hand and pull it toward them. This use is not possible in any way with conventional emergency cases that have rounded handles, for example.

Emergency cases have to be able to be carried easily and to be rapidly available. This also applies to these emergency cases according to the preferred embodiment: it can also be readily used when a first aider providing treatment is standing or kneeling beside the patient.

The doctor or other ambulance personnel or the like, as the first aider, can in particular set down the case on the ground beside a patient, then kneel down to the patient and pull the case over by stretching their arms. As a result, valuable time can be saved during treatment of the patient.

According to a preferred embodiment, in this aspect, the transition between the first portion and the second portion is rounded so that there are no disruptive corners. Alternatively or additionally, the transition between the second portion and the third portion is rounded so that there are no disruptive corners.

The second portion does not have to extend toward the inner longitudinal edge immediately from the point at which the first portion ends. Instead, it may be advantageous for the second portion of the handle to initially still extend in parallel with an outer longitudinal edge of the case shell in one section (i.e. partially) and to extend toward the inner longitudinal edge in a straight line only in a further section, wherein the section having the straight extension is not necessarily perpendicular to the outer longitudinal edge, but instead can be at a suitable angle of 15 to 30°, for example. As a result, the case not only has a pleasing appearance, but also, functionally, it is easier to grasp from above, and at the same time it is also ensured that the weight is balanced out when holding the case.

The third portion can extend centrally along the inner longitudinal edge. This is of course a particularly stable embodiment when the third portion, which is easily grasped by the user, extends centrally.

More preferably, each handle is then also formed to be symmetrical, i.e. the first portions and the second portions are mirror inverted relative to one another, and the third portion connects them. The mirror plane intersects the third portion into two halves.

When a handle is provided on each of the two case shells, with the handles preferably being symmetrical to one another, i.e. configured to be mirrored, the entire emergency case can be carried in a particularly stable manner. This is particularly applicable when the third portions of the handles contact one another.

It is preferably also provided that a stacking centering apparatus is formed at the end of each handle.

According to another preferred embodiment of the invention, stacking centering apparatuses are formed on the case shell and/or on the inner lid, with these preferably being arranged so as to be reversed.

In the present case, a “stacking centering apparatus” is understood such that a plurality of identical emergency cases or case shells can comprise stacking centering apparatuses that are complementary to one another, such that they can be stacked in a stable manner by means of an interlocking fit, if not even a latching fit. It is preferably provided here that a stacking centering apparatus is formed at the end of each handle.

The stacking centering apparatuses at the end of each handle, i.e. at the outer corners, of course correspond to the stacking centering apparatuses of a second emergency case, again at the outer corners. Stacking centering apparatuses can also be provided at the lower outer corners of the case shell. It may then be unimportant which way around the emergency cases that are being stacked are oriented.

A stacking centering apparatus can also be formed at each of the cover caps, which apparatus is then complementary to the stacking centering apparatuses of other cover caps or are complementary to one another at the ends of the handles.

The stacking centering apparatuses of a case shell are preferably complementary to one another at the upper outer corners (so as to be reversed). Identical case shells can then be easily stacked up.

Stacking centering apparatuses can also be provided on each inner lid, such that the case shells can be stacked individually.

According to a preferred embodiment of the invention, the inner lid is or can be latched to the associated case shell. This ensures increased protection for the material found in the case shell: when the inner lid is latched, it is particularly unlikely that it can fall out.

Furthermore, according to a preferred embodiment, it is provided that each inner lid can be opened by 90° (when the case is suitably at least partially open). Therefore, each emergency case half can be opened up to an extent, and the doctor or other user providing treatment as the first aider can easily remove the material found therein.

Alternatively or additionally, it is possible that, when the case is open, an inner lid (and preferably either of the two inner lids) is rotatable by 180°, such that it comes to rest on the other inner lid. In this way, the inner lid does not cause any disruption and material can be easily removed from this one emergency case half.

The two features of opening by 90° and rotating by 180° can both be implemented as desired, such that material can either be removed from both emergency case halves or the inner lid does not cause any disruption and material can be removed only from one emergency case half.

In these two embodiments of opening by 90° and rotating by 180°, it is preferably provided that the inner lids are latched to one another. These lids can in particular be latched to one another using the stacking centering apparatuses which are preferably provided anyway.

According to an additional aspect, the emergency case half comprises a case shell and a knob apparatus as well as a body which can be engaged behind, wherein the emergency case half can be connected to an identical case shell which comprises a knob apparatus and a body which can be engaged behind in order to provide an emergency case (in particular of the above-described type in at least one of the above-described embodiments, optionally also in one of the above-described preferred embodiments) in which each knob apparatus of one case shell can engage behind the body which can be engaged behind on the other case shell.

According to embodiments, the emergency case half comprises an inner lid. The case shell can be connected to an identically constructed case shell, in particular can be connected without tools. Said emergency case half can be transported on its own, for example by two users. If the emergency case is no longer required, they can be combined to form one emergency case.

Advantageous embodiments of the above-described emergency case according to the invention of course also apply to the emergency case halves, where applicable.

In particular, the ability to establish a connection to said rods and to the connection device that is complementary thereto without tools can be accomplished on the emergency case half.

A method for connecting two emergency case halves includes the fact that the two rods of the emergency case halves are inserted into the connection device of the respectively other emergency case half.

The use of an emergency case half is the use thereof on its own as an emergency case that is separately portable.

According to another preferred embodiment, it can be provided that the emergency case fluoresces. In this way, it is clearly visible at night, meaning that time can be saved during use.

For example, the emergency case can have walls that are configured to be at least partially fluorescent. This can be achieved when using plastics material for example by a fluorescent substance being added to the plastics granulate before injection molding or another molding process. This can for example be a photoluminescent substance, in particular a photoluminescent substance having a long time of persistence, by means of which a light density of 55/8 med/qm in accordance with DIN 67510 can be obtained, for example. For example, the fluorescent substance can be the substance known per se from EP 0 853 112 A1; in particular the substance can contain fluorescent particles, in particular with upconversion and/or downconversion. In this case, the particles also glow when there has not been any stimulation by ambient light for a prolonged period of time, for example in the event of a power outage. Residual radiation in a different frequency range is then emitted by upconversion or downconversion of these particles in the visible range such that the emergency case also remains clearly visible in this case.

Alternatively or additionally, the walls of the emergency case can be printed to be fluorescent and/or provided with an adhesive fluorescent film.

In addition, it is possible for the handle to fluoresce. A handle that is configured to be at least partially fluorescent and/or printed to be fluorescent and/or provided with an adhesive fluorescent film can therefore be provided. This can in particular be implemented on a handle insert. The rest of the handle, and potentially also the rest of the emergency case, then does not have to be configured to be fluorescent, which makes production more cost-effective.

This can include the fact that the handle alone glows. The handle is then particularly clearly visible, and so it is easier to grasp in darkness before pulling it over. This effect can likewise be obtained in the fluorescent walls by the handle fluorescing more brightly than the walls. The material for the handle can comprise a greater proportion of fluorescent particles than the rest of the emergency case.

The system according to the invention made up of a wall bracket and an emergency case includes retaining arms of the wall bracket formed to match to a handle of the emergency case such that the handle rests on each of the retaining arms by a lateral portion, and at the same time can be grasped by a central portion between the retaining arms in order to take the emergency case down from the wall bracket.

Unlike a simple hook, for example, in which the handle is retained centrally and has to be grasped by lateral portions in order to take down the case, providing retaining arms which leave a central portion of the emergency case free between the retaining arms makes it possible to grasp this central portion and to take the emergency case down from the wall bracket with one hand.

According to embodiments of the invention, the wall bracket comprises exactly two retaining arms. These arms can instead retain the emergency case on the handle quite far toward the outside, and therefore the central portion can be formed to be quite long. Alternatively, four retaining arms, six retaining arms, or the like can also be provided. Odd numbers of retaining arms are also possible in principle.

Preferably, each handle is arranged with two shoulder ends each on an upper outer corner of the case shell. The term “upper outer corner” relates to an upright position of the emergency case. Proceeding from each of the upper outer corners, the handle extends vertically upward in a first portion. (Since there are two outer corners, there are two first portions.) In a second portion preferably following the first portion, the handle extends toward the inner longitudinal edge of the case shell remote from the (outer) corners. (Since the first portion extends vertically upward, this should be understood to mean that the second portion likewise extends above the case shell upper face, wherein the extension toward the inner longitudinal edge of the case shell can also be formulated such that, in plan view, the second portion reaches the inner longitudinal edge.) A third portion of the handle connecting the two second portions further extends along the inner longitudinal edge. In other words, the handle extends vertically upward proceeding from a first upper outer corner in a first portion, then to the inner longitudinal edge in the second portion, then along the inner longitudinal edge in the third portion, then back toward the outer face of the case shell in another second portion, and then vertically downward toward the second outer corner.

An emergency case of this kind has the advantage that, by fastening the handle to the outer corners and owing to the presence of the third portion along the inner longitudinal edge, transport is facilitated, because the handle itself ensures that the case held by hand on the third portion is balanced out. In addition, the use makes it possible that, proceeding from the situation whereby the emergency case is lying flat on an outer face of the case shell or is lying with an outer face of the case shell on the ground or another surface, the user can grasp the third portion of the case with their hand and pull it toward them. This use is not possible in any way with conventional emergency cases that have rounded handles, for example.

Emergency cases have to be able to be carried easily and be rapidly available. This also applies to emergency cases according to the preferred embodiments which can be readily used when a first aider providing treatment is standing or kneeling beside the patient.

The doctor or other ambulance personnel or the like, as the first aider, can in particular set down the case on the ground beside a patient, then kneel down to the patient and pull the case over by stretching their arms. As a result, valuable time can be saved during treatment of the patient.

In connection with the wall bracket, this configuration of the handle is particularly advantageous because the emergency case can rest on the retaining arms by the second portions and at the same time can be grasped by the third portion.

According to a preferred embodiment, in this aspect, the transition between the first portion and the second portion is rounded so that there are no disruptive corners. Alternatively or additionally, the transition between the second portion and the third portion is rounded so that there are no disruptive corners.

The second portion does not have to extend toward the inner longitudinal edge immediately from the point at which the first portion ends. Instead, it may be advantageous for the second portion of the handle to initially still extend in parallel with an outer longitudinal edge of the case shell in one section (i.e. partially) and to extend toward the inner longitudinal edge in a straight line only in a further section, wherein the section having the straight extension is not necessarily perpendicular to the outer longitudinal edge, but instead can be at a suitable angle of 15 to 30°, for example. As a result, the case not only has a pleasing appearance, but also, functionally, it is easier to grasp from above, and at the same time it is also ensured that the weight is balanced out when holding the case.

The third portion can extend centrally along the inner longitudinal edge. This is of course a particularly stable embodiment when the third portion, which is easily grasped by the user, extends centrally.

More preferably, each handle is then also formed to be symmetrical, i.e. the first portions and the second portions are mirror inverted relative to one another, and the third portion connects them. The mirror plane intersects the third portion into two halves.

When a handle is provided on each of the two case shells, with the handles preferably being symmetrical to one another, i.e. configured to be mirrored, the entire emergency case can be carried in a particularly stable manner. This is particularly applicable when the third portions of the handles contact one another.

According to embodiments of the invention, it can be provided that the two case shells of an emergency case comprising two case shells are detachable from one another. This embodiment can be advantageous, inter alia when only one case shell is required, or when two first aiders working together on one patient have an emergency case of the above-mentioned type and each want to attend to them from one of the case shells using the material found therein.

Particularly preferably, in this aspect it is provided that the emergency case comprises two emergency case halves.

According to embodiments of the invention, one emergency case half can hang on the wall bracket by its handle. In this case, the other emergency case half can be opened up in this state, and the wall bracket comprises a stop in a foot region for the opened-up emergency case half. Therefore, this makes it possible to hang the emergency case on the wall bracket and at the same time to gain access to the material found in the emergency case, namely at least in the emergency case half that has been opened downward.

Particularly preferably, it is provided here that the two emergency case halves each comprise a case shell and an inner lid, wherein the emergency case halves are detachably connected, and wherein each inner lid can be locked to its case shell to produce a closed emergency case half.

By providing separable case shells and by providing the inner lid, the case shells can be transported on their own, i.e. can also be used on their own as an emergency case, and because of the lockable inner lid the material (plasters and dressings, disposable gloves, etc., as stated above) found in the respective emergency case half does not fall out in the process. The emergency case can thus be used flexibly: the two emergency case halves can remain connected; the first aider, doctor, or other user providing treatment then has a large emergency case containing a lot of material. When the emergency case halves are equipped identically, a particularly large amount of dressings are then provided, for example. When the emergency case halves are equipped differently, the contents can complement one another. When the emergency case halves are separated, they can each be used by two different users. Where necessary, they can be separated rapidly on site. When an emergency case is provided, two users can thus each use one emergency case half and can accordingly treat two patients simultaneously.

According to a preferred embodiment of this aspect, the emergency case halves are detachably connected solely by means located on the case shells. In this case, particular auxiliary means such as screws and the like can be omitted. In this embodiment, it is typically ensured that the emergency case halves can be detached from one another particularly rapidly, such that the above-described separation of the emergency case for two users is possible on site at short notice.

According to another preferred embodiment of this aspect, the emergency case comprises two identically constructed case shells. This can not only provide the advantage of symmetry, but also the connection means potentially located on the case shell can be configured to be complementary to one another. To produce an emergency case, two different case shells do not then need to be provided.

In this context, it is preferably provided that a rod and, coaxially therewith and so as to be reversed, a connection device for the rod of the other case shell are arranged on each case shell. The rod then just needs to be introduced into (pressed into or plugged into) the connection device that is preferably C-shaped in cross section in order to interconnect the emergency case halves, or, vice versa, the rods can be detached again when the connection device has sufficient elasticity. If this is done in both connection devices for the respective rods, the emergency case can be separated into the two emergency case halves.

More preferably, it is provided here that coaxially extending hinges for the respective inner lid of the case shell are arranged between the rod and the connection device, wherein the hinges of one case shell engage in gaps between the hinges for the other case shell. This can in particular be configured such that the gaps are completely closed except for a small clearance (where necessary, latching can even be provided); therefore, a practically continuous axis is provided both for the case shells and for the inner lid, which makes it possible to rotate the case shell halves relative to one another and to rotate the inner lid separately.

In the aspect of providing the stop for the opened-up emergency case half, the invention can be configured according to advantageous embodiments such that the stop makes it possible to open up the emergency case half at an angle of between 70° and 110°, preferably of between 85° and 95°, and more preferably of 90°. In this way, the stop can retain the opened-up emergency case half in a stable manner and at the same time can provide access to the interior thereof.

The above embodiment involving the detachability of the emergency case halves from one another can preferably be configured such that the two emergency case halves can already be detached from one another when the emergency case hanging on the wall bracket by an emergency case half is opened up until it reaches the stop. In this way, the first aider can rapidly take one emergency case half from the wall where necessary, even if the other emergency case half is supposed to stay on the wall for legal or operational reasons, so that material still remains in the room.

According to advantageous embodiments of the invention, the emergency case comprises ribs and the stop has complementary molded portions between the ribs. This allows for particularly stable retention.

The ribs can be reinforcement ribs on the outer wall of the emergency case, wherein each of these reinforcement ribs corresponds in position to a connection apparatus on the inner wall of the case shell for possibly inserting a partition element for forming compartments in the case shell. In this way, providing the connection apparatuses can be accompanied by providing the reinforcement ribs, and the case shell is not weakened. By means of the molded portions in the stop that are complementary to the ribs, the opened-up emergency case half is mounted in a stable manner.

According to other advantageous embodiments, the wall bracket comprises a fastener on a retaining arm. This makes it possible to avoid the entire emergency case being taken down too rapidly. Either the front emergency case half is opened up and, according to the above-described embodiment, the front emergency case half is detached from the rear emergency case half, or the fastener is opened, which, where necessary, only authorized persons provided with a key are capable of doing, or wherein the fastener at least prevents the emergency case halves from being taken down excessively rapidly, and, for example, also discourages unauthorized persons from doing this altogether.

According to advantageous embodiments in this regard, the fastener comprises a pin that engages in a cut-out in the emergency case and can be pulled out of the cut-out by a pull tab. This embodiment provides the fastener by way of very simple means.

In one configuration of this, a spring is provided in the fastener, wherein the pull tab can be pulled forward counter to the force of the spring in order to bring the pin out of engagement with the cut-out in the emergency case. The spring means that, when the pin is released again, it comes back into engagement with the cut-out in the emergency case. This can be useful when the aim is for the user not to be able to remove the emergency case too rapidly: they need to use both hands, because they need to pull the pull tab (forward) with one hand and use the other hand to lift the emergency case out of the wall bracket. This embodiment ensures improved retention of the emergency case, in particular in vehicles, in which driving means that forces act on the assembly (“jolting”).

A configuration without a spring may, however, be desirable when the emergency case is to be retained in the wall bracket and protected by a fastener, but is to be more rapidly removable.

The wall bracket according to the invention for an emergency case comprises a rear bar and at least two upper retaining arms for receiving an emergency case handle and comprises a stop in a foot region for making it possible to support an emergency case (an emergency case half) in the opened-up state.

The rear bar has the advantage of providing stability using few means, namely the two upper retaining arms that extend therefrom (for example, in a “T shape”). The entire assembly is particularly stable owing to the stop in the foot region.

According to advantageous embodiments, the wall bracket comprises exactly two retaining arms. This has the advantage of being a simple configuration.

According to advantageous embodiments, the retaining arms each comprise a seat for a portion of the emergency case handle and a retaining finger in front of the seat. Owing to the seat, the emergency case handle is retained in a stable manner, and the retaining finger prevents it from slipping down.

A fastener apparatus is preferably provided on one of the fingers, and more preferably it is then not provided on the other of the fingers. The fastener device has the above-mentioned advantage that the emergency case cannot be taken down from the wall bracket excessively rapidly, in particular not as a whole.

The use according to the invention of a system having the features of one of the above-described embodiments includes, when proceeding from the situation whereby the emergency case is retained on the wall bracket by the rear emergency case half:
Grasping the emergency case by the central portion of the handle of at least the rear emergency case half with a hand. (If the emergency case also has a handle on the front emergency case half, the central portions of both handles are grasped.)
Exerting a force in the vertical direction with one hand such that the emergency case is taken down from the wall bracket.

The other use according to claim 18, namely the use of an emergency case half of an emergency case in a system having the features according to claim 6 (stop; detachable emergency case halves), includes, when proceeding from the situation whereby the emergency case is retained on the wall bracket by the rear emergency case half:
first opening up the front emergency case half until it is retained by the stop;
then detaching the front emergency case half from the rear emergency case half.

In this case, the first aider can rapidly gain access to the emergency case half and use the material. For practical or potentially legal reasons, the rear emergency case half remains on the wall bracket where necessary, or it can be used by another first aider or other personnel, and in particular can be taken down by them.

The further use of an emergency case half of an emergency case in a system having the features according to claim 10 (pull tab on pin and spring for the pull tab), includes, when the emergency case is retained on the wall bracket by the rear emergency case half:
pulling the pull tab with one hand such that the pin comes out of engagement with the cut-out in the emergency case;
exerting a force on the emergency case in the vertical direction with the other hand such that the emergency case is taken down from the wall bracket. (In this case, the other hand preferably holds the emergency case by the handle, in particular by the central portion.)

The fact that two hands need to be used here can be considered an advantage in some circumstances. For instance, this can provide improved safety: if the first aider takes the pull tab of the fastener in their first hand and the emergency case in their second, the first hand can rapidly support an emergency case that has already been partially removed from below. This is particularly advantageous for use in an emergency vehicle, since particularly safe handling is ensured even when driving along.

According to embodiments, the emergency case comprises a first case shell and a second case shell, and a first knob apparatus on the first case shell, which can be brought from a rest position into a closed position and vice versa, and further comprises a second knob apparatus on the second case shell, which likewise can be brought from a rest position into a closed position and vice versa, wherein, when both knob apparatuses are in a given rest position, the emergency case is open and wherein, in the closed position of the first knob apparatus, the first knob apparatus engages behind a body formed on the second case shell by means of a rear-engaging element, and wherein, in the closed position of the second knob apparatus, the second knob apparatus engages behind a body formed on the first case shell by means of a rear-engaging element.

According to embodiments, the knob apparatuses are both provided on one case shell. Therefore, the stability of the assembly is increased. The emergency case thus has a particularly solid construction.

According to advantageous embodiments, each knob apparatus is rotatable. A rear-engaging element engaging behind a body can be implemented in a particularly simple manner when the rear-engaging element is rotated behind the body, i.e. the entire knob apparatus is rotatable.

In a preferred embodiment, it is provided that each knob apparatus is arranged on the front left when the associated case shell is opened upward (i.e. on the front right when the associated case shell is opened downward) and can be rotated counterclockwise from the rest position into the closed position. The knob apparatus arranged on the left and rotatable counterclockwise, corresponds to an outward movement, which can be easily performed from the wrist or elbow. The closure of the second knob apparatus, which is to be performed at the same time, from the front right counterclockwise downward can be performed practically synchronously from the right wrist downward. This closing process is particularly intuitive. The reverse opening process (right hand turns upward, the left downward) is also helpful.

According to another advantageous embodiment, each knob apparatus can comprise a knob, for which a geometric midpoint can be defined. An axis of rotation of the knob apparatus is then eccentric to the geometric midpoint of the knob.

In this specific aspect, the knob can be pivoted in a much improved manner and is thus easier to operate. This aspect can in turn constitute a separate invention, independent of the above-mentioned invention: An emergency case comprising a first case shell and a second case shell and comprising a knob apparatus on each case shell, wherein each knob apparatus comprises a knob, for which a geometric midpoint can be defined, and wherein an axis of rotation of the knob apparatus is eccentric to the geometric midpoint of the knob.

According to preferred embodiments of the invention and of all the other advantageous aspects, each knob apparatus comprises a cross-shaped knob. Unlike wheels, the cross shape is advantageous here because, on the basis of the cross shape, in particular also in connection with the eccentric mounting of the knob apparatus, it is easier to see what position the knob apparatus is in.

It is preferably provided here that legs of equal length of the cross shape are at an angle of 90° to the respectively adjacent leg. (The cross therefore has exactly four identical legs.) This makes it easier to operate.

According to embodiments, a (rotary) shaft of the knob apparatus can be formed on a first leg of the cross shape, as a result of which the above-mentioned embodiment of the eccentric mounting can be implemented.

The cross is therefore not rotated about the midpoint from which the four legs protrude, i.e. not about the geometric midpoint, but instead about one of the legs, such that the pivot range of the knob apparatus is enlarged, which in turn makes it easier to operate. This is particularly applicable in connection with the embodiment of the rotatability for closure in the clockwise direction for the knob apparatus arranged on the front left (relative to the open case shell).

Advantageously, it can be provided here that the rear-engaging element is formed on the leg of the cross shape opposite the first leg. The advantage of the cross shape is also clear here: the cross shape can be configured to be particularly stable owing to side walls on the legs, and this likewise makes it easier to operate.

According to embodiments of the aspect comprising the knob apparatus, the body behind which a knob apparatus engages or can engage can be formed as a lug. For this purpose, it preferably comprises a latching mechanism. Unlike a simple pin, the lug has the advantage of stability of the closure, since the two case shells are thus hooked into one another in a certain way. The hold is potentially further enhanced by the latching mechanism.

According to advantageous embodiments in this regard, there is also a stowed position in addition to the rest position and the closed position. This can be described such that each knob apparatus can be rotated from the rest position in a first rotational direction, preferably by 90°, to produce the closed position, and wherein each knob apparatus can be rotated from the rest position in a second rotational direction counter to the first rotational direction, preferably by 90°, to produce a stowed position.

This can have the advantage that the rear-engaging element causes less disruption, in order to make it easier to remove objects from the emergency case. This embodiment is particularly advantageous in connection with the eccentric mounting of the knob apparatus, and more particularly also in connection with the cross shape. When the cross is pivoted, it can cause particularly low disruption in the stowed position.

According to another preferred embodiment, at least one of the emergency case halves comprises a handle on the case shell. The emergency case can be particularly easily carried by means of a handle.

In one embodiment, both emergency case halves comprise a handle; the two emergency case halves can each be carried on their own after being detached from one another.

Alternatively, just one emergency case half comprises a handle. This can be useful if particularly important material is stored in the respective case shell and if material that is not always used is stored in the other case shell. The emergency case half containing the more important material can then be separated and the other emergency case half can be used only when needed. In this case, it is preferably provided on the case shell without a handle that cover caps are provided at the point at which the handle would otherwise engage or be attached.

It is preferably also provided that a stacking centering apparatus is formed at the end of each handle.

According to another preferred embodiment, stacking centering apparatuses are formed on the case shell and/or on the inner lid, with these preferably being arranged so as to be reversed.

In the present case, a “stacking centering apparatus” is understood such that a plurality of identical emergency cases or case shells can comprise stacking centering apparatuses that are complementary to one another, such that they can be stacked in a stable manner by means of an interlocking fit, if not a latching fit. It is preferably provided here that a stacking centering apparatus is formed at the end of each handle.

The stacking centering apparatuses at the end of each handle, i.e. at the outer corners, correspond to the stacking centering apparatuses of a second emergency case, again at the outer corners. Stacking centering apparatuses can also be provided at the lower outer corners of the case shell. It may then be unimportant which way around the emergency cases, that are being stacked, are oriented.

A stacking centering apparatus can also be formed at each of the cover caps that is then complementary to stacking centering apparatuses of other cover caps or are complementary to one another at the ends of the handles.

The stacking centering apparatuses of a case shell are preferably complementary to one another at the upper outer corners (so as to be reversed). Identical case shells can then be easily stacked up.

Stacking centering apparatuses can also be provided on each inner lid, such that the case shells can be stacked individually.

According to a preferred embodiment of the invention, the inner lid is or can be latched to the associated case shell. This ensures increased protection for the material found in the case shell: when the inner lid is latched, it is particularly unlikely that the material can fall out.

According to advantageous embodiments of all the above-mentioned aspects, an inner lid is provided on the associated case shell and is or can be latched thereto. This ensures increased protection.

Furthermore, according to a preferred embodiment, it is provided that each inner lid can be opened by 90° (when the case is suitably at least partially open). Therefore, each emergency case half can be opened up to an extent, and the doctor or other user providing treatment as the first aider can easily remove the material found therein.

Alternatively or additionally, it is possible that, when the case is open, an inner lid (and preferably either of the two inner lids) is rotatable by 180°, such that it comes to rest on the other inner lid. In this way, the inner lid does not cause any disruption and material can be easily removed from this one emergency case half.

The two features of opening by 90° and rotating by 180° can both be implemented as desired, such that material can either be removed from both emergency case halves or the inner lid does not cause any disruption and material can be removed only from one emergency case half.

In these two embodiments of opening by 90° and rotating by 180°, it is preferably provided that the inner lids are latched to one another. These lids can in particular be latched to one another using the stacking centering apparatuses which are preferably provided anyway.

According to advantageous embodiments, each emergency case half and/or at least one of the two emergency case halves comprises a case shell and a knob apparatus as well as a body which can be engaged behind, wherein the emergency case half can be connected to an identical case shell which comprises a knob apparatus and a body which can be engaged behind in order to provide an emergency case (in particular of the above-described type in at least one of the above-described embodiments, optionally also in one of the above-described preferred embodiments) in which each knob apparatus of one case shell can engage behind the body which can be engaged behind on the other case shell.

According to embodiments, the emergency case half comprises an inner lid. The case shell can be connected to an identically constructed case shell, in particular can be connected without tools. Said emergency case half can be transported on its own, for example by two users. If the emergency case is no longer required, they can be combined to form one emergency case.

Advantageous embodiments of the above-described emergency case according to the invention of course also apply to the emergency case halves, where applicable.

In particular, the ability to establish a connection to said rods and to the connection device that is complementary thereto without tools can be accomplished on the emergency case half.

A method for connecting two emergency case halves includes the feature that the two rods of the emergency case halves are inserted into the connection device of the respectively other emergency case half.

The use of an emergency case half is the use thereof on its own as an emergency case that is separately portable.

According to another preferred embodiment, it can be provided that the emergency case fluoresces. In this way, it is clearly visible at night, meaning that time can be saved during use.

For example, the emergency case can have walls that are configured to be at least partially fluorescent. This can be achieved when using plastics material for example by a fluorescent substance being added to the plastics granulate before injection molding or another molding process. This can for example be a photoluminescent substance, in particular a photoluminescent substance having a long time of persistence, by means of which a light density of 55/8 mcd/qm in accordance with DIN 67510 can be obtained, for example. For example, the fluorescent substance can be the substance known per se from EP 0 853 112 A1; in particular the substance can contain fluorescent particles, in particular with upconversion and/or downconversion. In this case, the particles also glow when there has not been any stimulation by ambient light for a prolonged period of time, for example in the event of a power outage. Residual radiation in a different frequency range is then emitted by upconversion or downconversion of these particles in the visible range such that the emergency case also remains clearly visible in this case.

Alternatively or additionally, the walls of the emergency case can be printed to be fluorescent and/or provided with an adhesive fluorescent film.

In addition, it is possible for the handle to fluoresce. A handle that is configured to be at least partially fluorescent and/or printed to be fluorescent and/or provided with an adhesive fluorescent film can therefore be provided. This can in particular be implemented on a handle insert. The rest of the handle, and potentially also the rest of the emergency case, then does not have to be configured to be fluorescent, which makes production more cost-effective.

This can include the feature that the handle alone glows. The handle is then particularly clearly visible, and so it is easier to grasp in darkness before pulling it over. This effect can likewise be obtained in the fluorescent walls by the handle fluorescing more brightly than the walls. The material for the handle can comprise a greater proportion of fluorescent particles than the rest of the emergency case.

The emergency case according to the invention thus comprises a handle for carrying the emergency case, and the handle comprises a main body. The handle further comprises a cavity for receiving an electronics module.

By providing an electronics module in a cavity in the handle, the handle can be configured such that the electronics module is particularly easy to insert, and is then inserted in a particularly stable manner, and can be particularly easily replaced where necessary. In addition, the handle is a component which is very often produced separately from other components of the emergency case (case shells). In this case, an electronics module can be integrated in the handle before the handle is fastened to the case shells in turn. This simplifies the integration of an electronics module of this kind in the emergency case. It is advantageous for the cavity to be provided in the main body of the handle. Other components can then be applied more easily.

In particular, according to an embodiment of the invention, the main body of the handle is formed as a plastics injection-molded part which forms a hollow profile in which the cavity is located. It being formed as a plastics injection-molded part, for example made of hard plastics material, can make the handle particularly stable. In this case, almost any shape can be selected for the cavity and the rest of the main body.

According to preferred embodiments, the main body of the handle is open to one side. In this way, an electronics module can be integrated from the open side without it being able to easily fall out again.

Furthermore, according to embodiments of the invention, a closure cap is or can be inserted into the main body of the handle. A closure cap of this kind can in particular prevent the electronics module from falling out and, where applicable, can improve its retention in the main body. In connection with the embodiment in which the main body is open to one side, the closure cap can be used to close off the open side and to thus retain the electronics module in a now closed cavity.

According to advantageous embodiments of said aspect, the closure cap comprises a strip-shaped main body. Such a main body can extend over a certain length of the handle. This can be advantageous if the strip-shaped main body is intended to bear information (logo of the manufacturer of the emergency case, instructions to the user, and the like). Irrespective of the strip shape, the closure cap can also be a different color from the main body and therefore make the emergency case easier to find. Furthermore, irrespective of the strip shape, the closure cap can be fluorescent or phosphorescent, so that the entire emergency case, the rest of which does not necessarily have to be fluorescent or phosphorescent, is easier to find.

According to embodiments of the invention, it can be provided that the closure cap can be pulled out in order to make it possible to insert or remove an electronics module. As a result, the emergency case can be equipped with an electronics module where necessary or can be produced individually in the production process of the handle as a whole, and then the closure cap can be removed again in order to insert the electronics module at another site, and in particular at another factory, and to later attach the handle to the case shells. An outdated (potentially defective) electronics module can also be removed in this way to make it possible to insert a new and preferably also more modern electronics module having new functions.

According to embodiments of the invention, it can be provided that the closure cap can be locked by a snap-in device. It then does not easily fall out of said emergency case.

In this case, it is preferably provided that studs on the closure cap are inserted into circular cut-outs. (Conversely, studs could be present on the main body and circular cut-outs on the closure cap. The two can also be implemented at the same time: studs and cut-outs on the closure cap and associated cut-outs and studs in the main body.) In particular, studs of this kind are easy to handle when using soft plastics material or rubber material. The closure cap can then generally also be removed and reinserted particularly easily.

According to embodiments of the invention, it can be provided that an electronics module is actually provided in the emergency case. The emergency case is thus capable of actually providing information.

According to preferred embodiments in this regard, the electronics module comprises at least one of:
a position sensor for detecting the position of the electronics module and therefore the emergency case;
a transmission apparatus for outputting signals for making it easier to find the electronics module and therefore the emergency case;
a receiving module for receiving a query signal.

Using the position sensor, the emergency case can be easily found where necessary. This may in particular also require the transmission apparatus, which then outputs information regarding the position. The transmission apparatus can send signals at regular intervals. Alternatively, the receiving module is additionally provided for receiving a query signal, which can then trigger the output of signals.

According to embodiments, the electronics module can be configured to draw energy from query signals in order to use it for sending a response signal. For instance, a user can, by means of a query apparatus (this can be or contain a smartphone or the like), send a signal to the electronics module, which then receives the query signal, draws the energy therefrom, obtains the information from the position sensor, and then sends a response signal via the transmission apparatus.

According to embodiments, the emergency case comprising the electronics module can comprise an electronics module comprising a memory, wherein information regarding the target contents of the case is stored in the memory, and wherein the electronics module is configured to output this information via a communication interface. A user can thus query what materials are intended to be contained in the emergency case, can check whether it actually contains these materials, and where necessary refill the emergency case again. This can in particular be used by maintenance personnel in hospitals and the like. In addition, anyone can read the memory using an application (or “app”) where necessary. In this case, as the communication interface the emergency case merely optionally comprises the transmission apparatus, which outputs a signal containing the information regarding the target contents at regular intervals. Where necessary, a receiving module for receiving a query signal may also be useful here.

According to embodiments of the emergency case, the emergency case comprises a case shell, to which the handle is attached. The emergency case can be particularly easily carried by means of a handle.

In one embodiment in this regard, the emergency case comprises two emergency case halves that are detachable from one another, and both emergency case halves comprise a handle; the two emergency case halves can each be carried on their own after being detached from one another.

Alternatively, just one emergency case half comprises a handle. This can be useful if particularly important material is stored in the respective case shell and if material that is not always used is stored in the other case shell. The emergency case half containing the more important material can then be separated and the other emergency case half can be used only when needed. In this case, it is preferably provided on the case shell without a handle that cover caps are provided at the point at which the handle would otherwise engage or be attached.

Preferably, each handle is arranged with two shoulder ends each on an upper outer corner of the case shell. The term “upper outer corner” relates to an upright position of the emergency case. Proceeding from each of the upper outer corners, the handle extends vertically upward in a first portion. (Since there are two outer corners, there are two first portions.) In a second portion preferably following the first portion, the handle extends toward the inner longitudinal edge of the case shell remote from the (outer) corners. (Since the first portion extends vertically upward, this should be understood to mean that the second portion likewise extends above the case shell upper face, wherein the extension toward the inner longitudinal edge of the case shell can also be formulated such that, in plan view, the second portion reaches the inner longitudinal edge.) A third portion of the handle connecting the two second portions further extends along the inner longitudinal edge. In other words, the handle extends vertically upward proceeding from a first upper outer corner in a first portion, then to the inner longitudinal edge in the second portion, then along the inner longitudinal edge in the third portion, then back toward the outer face of the case shell in the second portion, and then vertically downward toward the second outer corner.

An emergency case of this kind has the advantage that, by fastening the handle to the outer corners and owing to the presence of the third portion along the inner longitudinal edge, first of all, transport is facilitated, because the handle itself ensures that the case held by hand on the third portion is balanced out. In addition, the use makes it possible that, proceeding from the situation whereby the emergency case is lying flat on an outer face of the case shell or is lying with an outer face of the case shell on the ground or another surface, the user can grasp the third portion of the case with their hand and pull it toward them. This use is not possible in any way with conventional emergency cases that have rounded handles, for example.

Emergency cases have to be able to be carried easily and to be rapidly available. This also applies to these emergency cases according to the preferred embodiment: it can also be readily used when a first aider providing treatment is standing or kneeling beside the patient.

The doctor or other ambulance personnel or the like, as the first aider, can in particular set down the case on the ground beside a patient, then kneel down to the patient and pull the case over by stretching their arms. As a result, valuable time can be saved during treatment of the patient.

In said handle, the cavity can in particular be provided in the third portion of the handle, i.e. in particular a receptacle for an electronics module. As a result, this can provide enhanced retention for the electronics module in that an insert tab is provided on the closure cap of the above-mentioned type. Where required, the electronics module can be inserted and can be stabilized by the insert tab. Where required, the electronics module can also be attached to the insert tab and then inserted together with the closure cap.

According to a preferred embodiment, in the aspect relating to the shape of the handle, the transition between the first portion and the second portion is rounded so that there are no disruptive corners. Alternatively or additionally, the transition between the second portion and the third portion is rounded so that there are no disruptive corners.

The second portion does not have to extend toward the inner longitudinal edge immediately from the point at which the first portion ends. Instead, it may be advantageous for the second portion of the handle to initially still extend in parallel with an outer longitudinal edge of the case shell in one section (i.e. partially) and to extend toward the inner longitudinal edge in a straight line only in a further section, wherein the section having the straight extension is not necessarily perpendicular to the outer longitudinal edge, but instead can be at a suitable angle of 15 to 30°, for example. As a result, the case not only has a pleasing appearance, but also, functionally, it is easier to grasp from above, and at the same time it is also ensured that the weight is balanced out when holding the case.

The third portion can extend centrally along the inner longitudinal edge. This is of course a particularly stable embodiment when the third portion, which is easily grasped by the user, extends centrally.

More preferably, each handle is then also formed to be symmetrical, i.e. the first portions and the second portions are mirror inverted relative to one another, and the third portion connects them. The mirror plane intersects the third portion into two halves.

When the third portions of the handles contact one another, i.e. when a handle is provided on each of the two case shells, with the handles preferably being symmetrical to one another, i.e. configured to be mirrored, the entire emergency case can be carried in a particularly stable manner. This is particularly applicable when the third portions of the handles contact one another.

According to embodiments of the invention, it is provided that, when the emergency case comprises two emergency case halves, which each comprise a case shell and an inner lid, wherein the emergency case halves are detachably connected, and wherein each inner lid can be locked to its case shell to produce a closed emergency case half. This then provides the following advantage:

By providing separable case shells and by providing the inner lid, the case shells can be transported on their own, i.e. can also be used on their own as an emergency case, and because of the lockable inner lid the material (plasters and dressings, disposable gloves, etc., as stated above) found in the respective emergency case half does not fall out in the process. The emergency case can thus be used flexibly: the two emergency case halves can remain connected; the first aider, doctor, or other user providing treatment then has a large emergency case containing a lot of material. When the emergency case halves are equipped identically, a particularly large amount of dressings are then provided, for example. When the emergency case halves are equipped differently, the contents can complement one another. When the emergency case halves are separated, they can each be used by two different users. Where necessary, they can be separated rapidly on site. When an emergency case is provided, two users can thus each use one emergency case half and can accordingly treat two patients simultaneously.

According to a preferred embodiment in this regard, the emergency case halves are detachably connected solely by means located on the case shells. In this case, in particular auxiliary means such as screws and the like can be omitted. In this embodiment, it is typically ensured that the emergency case halves can be detached from one another particularly rapidly, such that the above-described separation of the emergency case for two users is possible on site at short notice.

According to another preferred embodiment, the emergency case comprises two identically constructed case shells. This can not only provide the advantage of symmetry, but also the connection means potentially located on the case shell can be configured to be complementary to one another. To produce an emergency case, two different case shells do not then need to be provided.

In this context, it is preferably provided that a rod and, coaxially therewith and so as to be reversed, a connection device for the rod of the other case shell are arranged on each case shell. The rod then just needs to be introduced into (pressed into or plugged into) the connection device that is preferably C-shaped in cross section in order to interconnect the emergency case halves, or, vice versa, the rods can be detached again when the connection device has sufficient elasticity. If this is done in both connection devices for the respective rods, the emergency case can be separated into the two emergency case halves.

More preferably, it is provided here that coaxially extending hinges for the respective inner lid of the case shell are arranged between the rod and the connection device, wherein the hinges of one case shell engage in gaps between the hinges for the other case shell. This can in particular be configured such that the gaps are completely closed except for a small clearance (where necessary, latching can even be provided); therefore, a practically continuous axis is provided both for the case shells and for the inner lid, which makes it possible to rotate the case shell halves relative to one another and to rotate the inner lid separately.

According to embodiments, the emergency case according to the invention comprises a first case shell and a second case shell, and a first knob apparatus on the first case shell, which can be brought from a rest position into a closed position and vice versa, and further comprises a second knob apparatus on the second case shell, which likewise can be brought from a rest position into a closed position and vice versa, wherein, when both knob apparatuses are in a given rest position, the emergency case is open and wherein, in the closed position of the first knob apparatus, the first knob apparatus engages behind a body formed on the second case shell by means of a rear-engaging element, and wherein, in the closed position of the second knob apparatus, the second knob apparatus engages behind a body formed on the first case shell by means of a rear-engaging element.

In this aspect, the invention proceeds from the fact that the knob apparatuses are both provided on one case shell. Therefore, the stability of the assembly is increased. The emergency case thus has a particularly solid construction.

According to a preferred embodiment of this aspect, each knob apparatus is rotatable. A rear-engaging element engaging behind a body can be implemented in a particularly simple manner when the rear-engaging element is rotated behind the body, i.e. the entire knob apparatus is rotatable.

In a preferred embodiment in this regard, it is in turn provided that each knob apparatus is arranged on the front left when the associated case shell is opened upward (i.e. on the front right when the associated case shell is opened downward) and can be rotated counterclockwise from the rest position into the closed position. When the knob apparatus is arranged on the left and can then be rotated counterclockwise, this corresponds to an outward movement, which can be easily performed from the wrist or elbow. The closure of the second knob apparatus, which is to be performed at the same time, from the front right counterclockwise downward can be performed practically synchronously from the right wrist downward. This closing process is particularly intuitive. The reverse opening process (right hand turns upward, the left downward) is also helpful.

According to another advantageous embodiment of the aspect comprising the knob apparatus, each knob apparatus can comprise a knob, for which a geometric midpoint can be defined. An axis of rotation of the knob apparatus is then eccentric to the geometric midpoint of the knob.

In this aspect, the knob can be pivoted in a much improved manner and is thus easier to operate. This aspect can in turn constitute a separate invention, independently of the above-mentioned invention: An emergency case comprising a first case shell and a second case shell and comprising a knob apparatus on each case shell, wherein each knob apparatus comprises a knob, for which a geometric midpoint can be defined, and wherein an axis of rotation of the knob apparatus is eccentric to the geometric midpoint of the knob.

According to preferred embodiments of the aspect comprising the knob apparatus, each knob apparatus comprises a cross-shaped knob. Unlike wheels, the cross shape is advantageous here because, on the basis of the cross shape, in particular also in connection with the eccentric mounting of the knob apparatus, it is easier to see what position the knob apparatus is in.

It is preferably provided here that legs of equal length of the cross shape are at an angle of 90° to the respectively adjacent leg. (The cross therefore has exactly four identical legs.) This makes it easier to operate.

According to embodiments, a (rotary) shaft of the knob apparatus can be formed on a first leg of the cross shape, as a result of which the above-mentioned embodiment of the eccentric mounting can be implemented.

The cross is therefore not rotated about the midpoint from which the four legs protrude, i.e. not about the geometric midpoint, but instead about one of the legs, such that the pivot range of the knob apparatus is enlarged, which in turn makes it easier to operate. This is particularly applicable in connection with the embodiment of the rotatability for closure in the clockwise direction for the knob apparatus arranged on the front left (relative to the open case shell).

Advantageously, it can be provided here that the rear-engaging element is formed on the leg of the cross shape opposite the first leg. The advantage of the cross shape is also clear here: the cross shape can be configured to be particularly stable owing to side walls on the legs, and this likewise makes it easier to operate.

According to embodiments in all the aspects comprising the knob apparatus, the body behind which a knob apparatus engages or can engage can be formed as a lug. For this purpose, it preferably comprises a latching mechanism. Unlike a simple pin, the lug has the advantage of stability of the closure, since the two case shells are thus hooked into one another in a certain way. The hold is potentially further enhanced by the latching mechanism.

According to embodiments of the aspect comprising the knob apparatus, there is also a stowed position in addition to the rest position and the closed position.

This can be described such that each knob apparatus can be rotated from the rest position in a first rotational direction, preferably by 90°, to produce the closed position, and wherein each knob apparatus can be rotated from the rest position in a second rotational direction counter to the first rotational direction, preferably by 90°, to produce a stowed position.

This can have the advantage that the rear-engaging element causes less disruption, in order to make it easier to remove objects from the emergency case. This embodiment is particularly advantageous in connection with the eccentric mounting of the knob apparatus, and more particularly also in connection with the cross shape. When the cross is pivoted, it can cause particularly low disruption in the stowed position.

It is preferably also provided that a stacking centering apparatus is formed at the end of each handle.

According to other embodiments, stacking centering apparatuses can be formed on the case shell and/or on the inner lid, with these preferably being arranged so as to be reversed.

In the above-described embodiment, the stacking centering apparatuses can in particular be arranged at the end of each handle (“on an upper outer corner of the case shell”).

In the present case, a “stacking centering apparatus” is understood such that a plurality of identical emergency cases or case shells can comprise stacking centering apparatuses that are complementary to one another, such that they can be stacked in a stable manner by means of an interlocking fit, if not even a latching fit. It is preferably provided here that a stacking centering apparatus is formed at the end of each handle.

The stacking centering apparatuses at the end of each handle, i.e. at the outer corners, of course correspond to the stacking centering apparatuses of a second emergency case, again at the outer corners. Stacking centering apparatuses can also be provided at the lower outer corners of the case shell. It may then be unimportant which way around the emergency cases that are being stacked are oriented.

A stacking centering apparatus can also be formed at each of the cover caps, which apparatus is then complementary to the stacking centering apparatuses of other cover caps or are complementary to one another at the ends of the handles.

The stacking centering apparatuses of a case shell are preferably complementary to one another at the upper outer corners (so as to be reversed). Identical case shells can then be easily stacked up.

Stacking centering apparatuses can also be provided on each inner lid, such that the case shells can be stacked individually.

According to a preferred embodiment of the invention, the inner lid is or can be latched to the associated case shell. This ensures increased protection for the material found in the case shell: when the inner lid is latched, it is particularly unlikely that it can fall out.

Furthermore, according to a preferred embodiment, it is provided that each inner lid can be opened by 90° (when the case is suitably at least partially open). Therefore, each emergency case half can be opened up to an extent, and the doctor or other user providing treatment as the first aider can easily remove the material found therein.

Alternatively or additionally, it is possible that, when the case is open, an inner lid (and preferably either of the two inner lids) is rotatable by 180°, such that it comes to rest on the other inner lid. In this way, the inner lid does not cause any disruption and material can be easily removed from this one emergency case half.

The two features of opening by 90° and rotating by 180° can both be implemented as desired, such that material can either be removed from both emergency case halves or the inner lid does not cause any disruption and material can be removed only from one emergency case half.

In these two embodiments of opening by 90° and rotating by 180°, it is preferably provided that the inner lids are latched to one another. These lids can in particular be latched to one another using the stacking centering apparatuses which are preferably provided anyway.

The emergency case half according to the invention comprises a handle for carrying the emergency case, wherein the handle comprises a main body, wherein the handle (in particular the main body) comprises a cavity for receiving an electronics module. All the above-described preferred embodiments relating to the emergency case are also applicable to the emergency case half, provided that they are applicable to only one single half.

Advantageously in this regard, it is provided that the emergency case half comprises a case shell and a knob apparatus as well as a body which can be engaged behind, wherein the emergency case half can be connected to an identical case shell which comprises a knob apparatus and a body which can be engaged behind in order to provide an emergency case (in particular of the above-described type in at least one of the above-described embodiments, optionally also in one of the above-described preferred embodiments) in which each knob apparatus of one case shell can engage behind the body which can be engaged behind on the other case shell.

According to embodiments, the emergency case half comprises an inner lid. The case shell can be connected to an identically constructed case shell, in particular can be connected without tools. Said emergency case half can be transported on its own, for example by two users. If the emergency case is no longer required, they can be combined to form one emergency case.

The ability to establish a connection to said rods and to the connection device that is complementary thereto without tools can be accomplished on the emergency case half.

A method for connecting two emergency case halves includes the fact that the two rods of the emergency case halves are inserted into the connection device of the respectively other emergency case half.

The use of an emergency case half is the use thereof on its own as an emergency case that is separately portable.

According to another preferred embodiment, it can be provided that the emergency case fluoresces. In this way, it is clearly visible at night, meaning that time can be saved during use.

For example, the emergency case can have walls that are configured to be at least partially fluorescent. This can be achieved when using plastics material for example by a fluorescent substance being added to the plastics granulate before injection molding or another molding process. This can for example be a photoluminescent substance, in particular a photoluminescent substance having a long time of persistence, by means of which a light density of 55/8 mcd/qm in accordance with DIN 67510 can be obtained, for example. For example, the fluorescent substance can be the substance known per se from EP 0 853 112 A1; in particular the substance can contain fluorescent particles, in particular with upconversion and/or downconversion. In this case, the particles also glow when there has not been any stimulation by ambient light for a prolonged period of time, for example in the event of a power outage. Residual radiation in a different frequency range is then emitted by upconversion or downconversion of these particles in the visible range such that the emergency case also remains clearly visible in this case.

Alternatively or additionally, the walls of the emergency case can be printed to be fluorescent and/or provided with an adhesive fluorescent film.

In addition, it is possible for the handle to fluoresce. A handle that is configured to be at least partially fluorescent and/or printed to be fluorescent and/or provided with an adhesive fluorescent film can therefore be provided. This can in particular be implemented on a handle insert. The rest of the handle, and potentially also the rest of the emergency case, then does not have to be configured to be fluorescent, which makes production more cost-effective.

This can include the fact that the handle alone glows. The handle is then particularly clearly visible, and so it is easier to grasp in darkness before pulling it over. This effect can likewise be obtained in the fluorescent walls by the handle fluorescing more brightly than the walls. The material for the handle can comprise a greater proportion of fluorescent particles than the rest of the emergency case.

This embodiment can in particular be provided in that, in the embodiment of the handle comprising the closure cap, for example comprising the strip-shaped main body of the closure cap, this closure cap fluoresces more brightly or comprises a greater proportion of fluorescent particles than the main body of the handle, which can be injection-molded from the same material or otherwise produced in the same way as the case shells, for example.

The use according to the invention of an emergency case comprising the closure cap that can be pulled out contains the steps of:
removing the closure cap from the main body of the handle;
inserting an electronics module into the cavity in the handle;
closing the handle again by inserting the closure cap into the main body of the handle.

In this use, the emergency case is retrofitted with a functioning and, where necessary, modern (current) electronics module.

According to embodiments in this regard, the emergency case is already provided with an electronics module in the handle. Before inserting an electronics module into the cavity, the previous electronics module is removed from the cavity. The involves replacing an outdated and potentially defective electronics module with a new electronics module.

The emergency case according to the invention comprises in a possible embodiment two case shells and a seal, wherein the seal is formed by a sealing material, which is materially bonded to one of the case shells, and the seal is in contact with both case shells in the closed state of the emergency case.

The material bonding of the sealing material to the case shell has several advantages. The risk of the seal becoming detached from the case shell, in particular the risk of the seal falling out of a receiving groove in the open state, is thus minimized and the emergency case remains sealed even when it has already been used multiple times. In addition, the sealing of the emergency case by the material bond between the seal and the case shell is increased in comparison with merely inserting a ready-molded seal into a receiving groove in the case shell. In this way, sealing of the interior of the emergency case from the external environment in accordance with IP 67 can be achieved. In this context, it is also particularly preferred for the corresponding case shell to exert a mechanical pressure on the seal, which is deformed thereby, and this enlarges the contact surface between the seal and the case shells and thus increases the strength of the sealing. It is also conceivable for at least one seal to be arranged on both case shells, which seal is in contact with the other case shell in the closed state of the emergency case, for example by means of projecting elements formed on the case shell. Likewise, seals which are opposite one another and mutually contact one another in the closed state of the emergency case are conceivable.

According to advantageous embodiments of the invention, the sealing material is a flexible foam. Such a foam can deform particularly easily and repeatedly owing to the mechanical pressure exerted on the seal by the corresponding case shell and adjusts to different geometries. This enlarges the contact surface between the seal and the case shells to a greater extent, meaning that the strength of the sealing of the interior of the emergency case from the external environment is increased further.

According to an advantageous embodiment in this regard, the flexible foam is a polyurethane foam which is crosslinked at room temperature and results from a reaction of a polyol or a mixture of polyols and methylene diphenyl diisocyanate or one of its derivatives. A seal of which the sealing material is a flexible polyurethane foam of this kind is particularly beneficial, temperature-resistant, light, and stable.

According to advantageous embodiments, the case shells consist of acrylonitrile butadiene styrene plastics material, meaning that the emergency case remains rigid and tough and also has high hardness even at low temperatures, and so the contents of the emergency case are particularly well protected, since possible malfunctions and damage to the emergency case are prevented. In addition, an acrylonitrile butadiene styrene plastics material is easy to process owing to its thermoplasticity and it is easy to obtain a material bond to a sealing material that is based on plastics material. In this context, it is particularly advantageous for the sealing material to be a polyurethane foam which is crosslinked at room temperature and has been formed directly on the case shell by a reaction of a polyol or a mixture of polyols and methylene diphenyl diisocyanate or one of its derivatives, since the sealing material is thus directly materially bonded to the case shell by a chemical reaction.

An emergency case according to the invention preferably comprises a pressing element, which is formed on or fastened to a case shell opposite the seal, and at the same time is configured and positioned such that it exerts mechanical pressure on the seal in the closed state of the emergency case. The seal deforms to a greater extent owing to the additional mechanical pressure. This in turn enlarges the contact surface between the seal and the case shell yet further, meaning that the strength of the sealing of the interior of the emergency case from the external environment is increased yet further. In this context, it is particularly preferred for the pressing element to have a contour that corresponds to the outline of the seal, such that the seal is spatially continuous as far as possible and a constantly high mechanical pressure is applied thereto by the pressing element. It is particularly advantageous for the pressing element to have a narrower contour than the seal, since then the pressing element can project into the seal, and not only the upper surfaces of the pressing element, but also the lateral surfaces, can come into contact with the seal.

If one of the case shells comprises a receiving groove running along its edge and if the seal is arranged in this receiving groove, the boundary surface over which the material bond of the seal to the case shell is produced is enlarged and the strength of the material bond is increased. This increases the load-bearing capacity of the material bond and also the sealing of the emergency case in the closed state. In conjunction with a pressing element that has a lower width than the receiving groove, the seal can also be deformed within the receiving groove such that both the inner surfaces of the receiving groove and the lateral surfaces of the pressing element are in contact with the seal.

According to an advantageous embodiment in this regard, the seal projects beyond the peripheral receiving groove. As a result, contact between the seal and the corresponding case shell is simplified, since this does not need to have any dedicated pressing elements, for example, and this increases the freedom of choice when configuring the geometry of the case shell. In a corresponding configuration of the two case shells, a wider planar surface of the elements of the corresponding case shell that are supposed to come into contact with the seal can for example thus also be selected, and the corresponding case shell can still apply mechanical pressure to the seal in the closed state and accordingly deform it.

According to another advantageous embodiment, one of the case shells comprises partition elements for forming sub-sections which can be inserted into the case shell, wherein additional seals are arranged on the case shell opposite the insertable partition elements and correspond, at least in one direction, to the contour of the sub-sections that can be formed by the insertable partition elements. In this case, the additional seals are in contact with the longitudinal edges of the partition elements in the closed state of the emergency case. In this way, variable sub-sections can be produced by rearranging the partition elements, wherein the different sub-sections are sealed from other sections by contacting the longitudinal edges with the additional seals in the closed state of the emergency case. As a result, the sealed sub-sections are not only protected against contamination originating from the outside, such as germs or dirt particles, but also against contamination originating from other sections of the emergency case. For example, this could be undesired active substances stored in another section, or moisture, which originates from a stored eyewash kit, for example. Introduction of contamination of this kind could compromise the safe operation of first aid materials or first aid devices which are stored in the sealed sub-section.

According to an advantageous embodiment in this regard, the additional seals are arranged such that they correspond to the contours of the sub-sections that can be formed by the insertable partition elements, wherein the additional seals are in contact with longitudinal edges of the insertable partition elements in the closed state of the emergency case. Here, a grid-shaped arrangement of the additional seals along a plurality of imaginary horizontal and vertical lines is particularly preferred here, wherein the arrangement of the additional seals corresponds exactly to the grid formed by the insertable partition elements when they form the maximum number of variable sub-sections; for example, the lines which each both form grids divide the area into rectangles in the same way. In this way, each one of the individual variable sub-sections which can be produced by rearranging the partition elements is sealed. It is also possible to retroactively modify the sub-sections, for example for the customer to modify them on site, wherein any retroactively modified sub-section is also sealed both from the other sub-sections and from the external environment.

According to another advantageous embodiment, fixed partition elements for forming fixed sub-sections are formed on one of the case shells, wherein the seal is arranged on a longitudinal edge of one of the fixed partition elements. The longitudinal edge preferably comprises a receiving groove for receiving the seal and the seal is arranged in this receiving groove. As a result, one sub-section of the emergency case is sealed from the other sections and the external environment. As a result, this sub-section is not only protected against contamination originating from the outside, such as germs or dirt particles, but also against contamination originating from other sections of the emergency case.

According to another advantageous embodiment, fixed partition elements for forming fixed sub-sections are formed on one of the case shells, wherein an additional seal is arranged on a longitudinal edge of one of the fixed partition elements and is materially bonded to the longitudinal edge. In this context, the longitudinal edge preferably comprises a receiving groove and the seal is arranged in this receiving groove. In this way, the additional seal generates additional sealing, which additionally seals the fixed sub-section of the emergency case from the external environment while it is still sealed from the rest of the interior. As a result, the fixed sub-section is doubly protected against contamination originating from the outside, while protection against contamination originating from other sections of the emergency case is maintained.

If the emergency case is filled with at least one of plasters and dressings, disposable gloves, first aid scissors and tweezers, hygienic face masks, hygienic clothing, emergency showers and eyewash kits, disinfectants and dispensers therefor, defibrillators and accessories, or respiratory masks, they can be stored so as to be easy to access and simultaneously protected against contamination.

The double emergency case according to the invention comprises two emergency cases according to the invention, which are detachably connected to one another, wherein a case shell for forming a closed emergency case can be locked to the corresponding case shell of the same emergency case in each case. In this way, the two emergency case halves can remain connected, while the locking of the case shells to the respectively corresponding case shell of one of the emergency cases ensures that the seals are in contact with both case shells in the closed state and the contents of the individual emergency cases are protected against contamination. If both emergency case halves are connected to form a double emergency case, the first aider, doctor, or other user providing treatment has a large emergency case containing a lot of material. When the emergency cases are equipped identically, a particularly large amount of dressings are then provided, for example. When the emergency cases are equipped differently, the contents can complement one another. When the emergency cases are separated, the improved protection against contamination is still ensured and the emergency cases can be used by two different users. Where necessary, they can be separated rapidly on site. When a double emergency case is provided, two users can thus each use one emergency case and can accordingly treat two patients simultaneously with first aid materials that have been protected against contamination up to that point.

The method according to the invention for producing an emergency case includes providing two case shells and applying a flowable sealing component to one of the case shells, wherein the flowable sealing component is selected such that, after being applied, it both forms a chemical bond with the surface of the case shell and reacts to form a solid sealing material, by means of which the resulting sealing material is materially bonded to the case shell and forms a seal. Furthermore, the method according to the invention includes connecting the two case shells to form an emergency case in such a way that the seal is in contact with both case shells in the closed state of the emergency case.

In this context, the flowable sealing component is preferably applied using a machine. This rules out any germs from being introduced by a human while manually inserting a ready-molded seal, which reduces the germ load of the interior of the emergency case. This is particularly advantageous in relation to the subsequent use of the emergency case. The risk of a ready-molded seal being inaccurately or incorrectly inserted owing to human inaccuracies or mistakes is also ruled out. A mistake of this kind can e.g. involve the seal becoming kinked, which can result in unsealed regions. In addition, different sizes and shapes of seals do not need to be kept available in order to be able to produce different emergency cases. Furthermore, the time required for introducing the seal is reduced, wherein the amount of time taken to manually insert the seal, as used in the prior art, is approximately 2 minutes per emergency case. Moreover, manufacturing tolerances are compensated for in an improved manner, since the flowable sealing component levels itself after the sealing material is applied and before it forms, which results in a more uniform geometry and in particular height of the resulting seal on the case shell. In the method, a flowable sealing component is preferably used which reacts to form a sealing material at room temperature, which foams to form a uniform seal. In this context, it is conceivable for a chemical reaction between two constituents of the flowable sealing component to initiate the foaming. For example, this can be implemented in that the individual constituents of the sealing component are mixed together only right before the moment at which they are applied and the corresponding chemical reaction is initiated by the mixing. Using the method according to the invention, a seamlessly closed seal can be produced which, taking into account a given maximum application thickness, can have practically any conceivable contour and geometry. Even though some of the above-mentioned advantages can be attributed to applying the flowable sealing component using a machine, manual application is also conceivable and is explicitly included in the invention. Compared with the prior art, this even results in a plurality of the above-mentioned advantages, such as compensating for manufacturing tolerances, the strong material bond, and the option of being able to produce different emergency cases without needing to keep different seals available.

According to an advantageous embodiment in this regard, one of the case shells comprises a receiving groove running along the edge and the flowable sealing component is applied within the receiving groove. In this way, the flowable sealing component is prevented from flowing to undesired points on the case shell. In particular in conjunction with the self-leveling of the sealing component, this results in a particularly uniform geometry and height of the seal that is formed.

If the case shells consist of acrylonitrile butadiene styrene plastics material and the flowable sealing component contains both a polyol or a mixture of polyols and methylene diphenyl diisocyanate or one of its derivatives, a polyurethane foam that crosslinks just at room temperature can be obtained which is extremely stable against influences such as moisture, dust, and temperature.

The phosphorescent material and the production and/or composition described in this patent application, in particular with regard to particle size and/or chemical composition and/or physical properties, of the phosphorescent material and/or its processing, in particular in synthetic materials, can advantageously also be used for other objects for which luminescence is advantageous. These include, for example, switches, in particular light switches, sockets, warning signs such as warning signs, direction indicators, guidance signs for evacuation in an emergency. This also includes handles such as hand grips, window handles and cabinet handles so that they can be gripped and operated safely even in the dark.

An embodiment that has at least one of the following aspects is particularly advantageous:
1. An emergency case (200) comprising a first case shell and a second case shell, and comprising a first knob apparatus (90a) on the first case shell, which can be brought from a rest position into a closed position and vice versa, and comprising a second knob apparatus (90b) on the second case shell, which likewise can be brought from a rest position into a closed position and vice versa, wherein, when both knob apparatuses (90a, 90b) are in a given rest position, the emergency case (200) is open and wherein, in the closed position of the first knob apparatus (90a), the first knob apparatus (90a) engages behind a body (98) formed on the second case shell by means of a rear-engaging element (96), and wherein, in the closed position of the second knob apparatus (90b), the second knob apparatus (90b) engages behind a body (98) formed on the first case shell by means of a rear-engaging element (96).
2. The emergency case (200) according to aspect 1, in which the two case shells (10, 10′) are detachable from one another.
3. The emergency case (200) according to aspect 2, comprising two emergency case halves, which each comprise a case shell (10, 10′) and an inner lid (26), wherein the emergency case halves are detachably connected, and wherein each inner lid can be locked to its case shell (10, 10′) to produce a closed emergency case half.
4. The emergency case (200) according to any one of the preceding aspects 1 to 3, in which each knob apparatus (90a, 90b) is rotatable.
5. The emergency case (200) according to any one of the preceding aspects 1 to 4, in which each knob apparatus (90a, 90b) is arranged on the front left when the associated case shell is opened upward and can be rotated clockwise from the rest position into the closed position.
6. The emergency case (200) according to any one of the preceding aspects 1 to 5, in which each knob apparatus (90a, 90b) comprises a knob (92), for which a geometric midpoint (M) can be defined, and wherein an axis of rotation of the knob apparatus is eccentric to the geometric midpoint (M) of the knob (92).
7. The emergency case (200) according to any one of the preceding aspects 1 to 6, in which each knob apparatus (90a, 90b) comprises a cross-shaped knob (92), wherein legs of equal length are preferably at an angle of 90° to the respectively adjacent leg.
8. The emergency case (200) according to aspect 7, in which a shaft (94) of the knob apparatus is formed on a first leg (93a) of the cross shape.
9. The emergency case (200) according to aspect 8, in which the rear-engaging element (96) is formed on the leg (93b) of the cross shape opposite the first leg (93a).
10. The emergency case (200) according to any one of the preceding aspects 1 to 9, in which the body behind which a knob apparatus engages or can engage is formed as a lug (98) and preferably comprises a latching mechanism.
11. The emergency case (200) according to any one of the preceding aspects 1 to 10, in which each knob apparatus (90a, 90b) can be rotated from the rest position in a first rotational direction, preferably by 90°, to produce the closed position, and in which each knob apparatus (90a, 90b) can be rotated from the rest position in a second rotational direction counter to the first rotational direction, preferably by 90°, to produce a stowed position.
12. The emergency case (200) according to any one of the preceding aspects 1 to 11, in which a handle (12) is formed at least on one case shell (10), which handle, in an upright position of the emergency case (100, 200), is arranged on an upper outer corner of the case shell (10) in each case at two shoulder ends of the handle (12), extends vertically upward in a first portion (16a, 16b), and extends toward the inner longitudinal edge (22) of the case shell (10) remote from the corners in a second portion (18a, 18b), and wherein a third portion (20) of the handle (12) connecting the two second portions (18a, 18b) extends along the inner longitudinal edge (12).
13. The emergency case (200) according to any one of the preceding aspects 1 to 12, which is filled with at least one of: plasters and dressings, disposable gloves, first aid scissors and tweezers, hygienic face masks, hygienic clothing, emergency showers and eyewash kits, disinfectants and dispensers therefor, defibrillators and accessories, respiratory masks.
14. An emergency case half comprising a case shell (10) which comprises a knob apparatus (90a) and a body (98) which can be engaged behind, wherein the emergency case half can be connected to an identical case shell (10′) which comprises a knob apparatus (90b) and a body (98) which can be engaged behind in order to provide an emergency case (200) in which each knob apparatus (90a, 90b) of one case shell can engage behind the body (98) which can be engaged behind on the other case shell.
15. The emergency case half according to aspect 14, which is filled with at least one of: plasters and dressings, disposable gloves, first aid scissors and tweezers, hygienic face masks, hygienic clothing, emergency showers and eyewash kits, disinfectants and dispensers therefor, defibrillators and accessories, respiratory masks.

An embodiment that has at least one of the following aspects is particularly advantageous:
1. An emergency case comprising at least one case shell and a handle formed on the case shell, the handle arranged on upper outer corners of the case shell of the emergency case in an upright position at two shoulder ends of the handle, the handle extending vertically upward in two first portions, and extending toward an inner longitudinal edge of the case shell remote from the corners in two second portions, and wherein a third portion of the handle connecting the two second portions extends along the inner longitudinal edge.
2. The emergency case according to aspect 1, wherein a transition between the first portion and the second portion and/or the transition between the second portion and the third portion is rounded.
3. The emergency case according to aspects 1 or 2, wherein the second portion of the handle extends partially in parallel with an outer longitudinal edge of the case shell and then extends further toward the inner longitudinal edge in a straight line.
4. The emergency case according to aspect 1, wherein the third portion extends centrally along the inner longitudinal edge.
5. The emergency case according to aspect 4, wherein each handle is formed to be symmetrical.
6. The emergency case according to aspect 1, comprising two case shells that are complementary to one another.
7. The emergency case according to aspect 6, comprising a handle on both case shells.
8. The emergency case according to aspect 7, wherein the third portions of the two handles contact one another.
9. The emergency case according to aspects 7 or 8, wherein a stacking centering apparatus is formed at an end of each handle.
10. The emergency case according to aspect 6, comprising a handle on only one of the two case shells, wherein a cover cap is provided on each of the outer corners of the case shell without a handle.
11. The emergency case according to aspect 10, wherein a stacking centering apparatus is formed at the end of the handle and wherein a second stacking centering apparatus is formed on each of the cover caps.
12. The emergency case according to aspect 11, wherein the two stacking centering apparatuses of a case shell are complementary to one another.
13. The emergency case according to aspect 1, wherein each case shell is part of an emergency case half, which is detachably connected to the other emergency case half.
14. The emergency case according to aspect 1, wherein each case shell has an inner lid.
15. The emergency case according to aspect 14, comprising stacking centering apparatuses on each inner lid.
16. The emergency case according to aspect 1, comprising walls that are configured to be at least partially fluorescent and/or printed to be fluorescent and/or provided with fluorescent stickers.
17. The emergency case according to aspect 16, wherein the fluorescence is photoluminescence.
18. The emergency case according to aspect 17, wherein the photoluminescence is with upconversion and/or downconversion.
19. The emergency case according to aspect 1, wherein the handle is configured to be at least partially fluorescent and/or printed to be fluorescent and/or provided with fluorescent stickers.
20. The emergency case according to aspect 19, wherein the fluorescence is photoluminescence.
21. The emergency case according to aspect 20, wherein the photoluminescence is with upconversion and/or downconversion.
22. The emergency case according to aspects 16 and aspect 19, wherein the handle fluoresces more brightly than the walls.
23. The emergency case according to aspect 1, filled with at least one of: plasters and dressings; disposable gloves; first aid scissors and tweezers; hygienic face masks; hygienic clothing; emergency showers and eyewash kits; disinfectants and dispensers therefor; defibrillators and accessories; respiratory masks.
24. Use of the emergency case according to aspect 1.
25. The use of the emergency case according to aspect 24 by a person providing medical treatment in an emergency medical situation, comprising:
grasping, by a hand of the person, the third portion of the emergency case, wherein the emergency case is lying with an outer face of the case shell on the ground or a surface; and
pulling the emergency case toward the person.

An embodiment that has at least one of the following aspects is particularly advantageous:
Aspect 1: An emergency case comprising at least one case shell, wherein a handle (12) is formed on the case shell, which handle, in an upright position of the emergency case, is arranged on an upper outer corner of the case shell in each case at two shoulder ends of the handle, extends vertically upward in a first portion, and extends toward the inner longitudinal edge of the case shell remote from the corners in a second portion, and wherein a third portion of the handle connecting the two second portions extends along the inner longitudinal edge.
Aspect 2: The emergency case according to Aspect 1, in which the transition between the first portion and the second portion and/or the transition between the second portion and the third portion is rounded.
Aspect 3: The emergency case according to Aspect 1 or 2, in which the second portion of the handle extends partially in parallel with an outer longitudinal edge of the case shell and then extends further toward the inner longitudinal edge in a straight line.
Aspect 4: The emergency case according to any one of Aspects 1 to 3, in which the third portion extends centrally along the inner longitudinal edge.
Aspect 5: The emergency case according to Aspect 4, in which each handle is formed to be symmetrical.
Aspect 6: The emergency case according to any one of Aspects 1 to 5, comprising two case shells (10) that are complementary to one another.
Aspect 7: The emergency case according to Aspect 6, comprising a handle on both case shells (10).
Aspect 8: The emergency case according to Aspect 7, wherein the third portions of the two handles contact one another.
Aspect 9: The emergency case according to Aspect 7 or 8, wherein a stacking centering apparatus is formed at the end of each handle.
Aspect 10: The emergency case according to Aspect 6, comprising a handle on only one of the two case shells, wherein a cover cap (42a, 42b) is provided on each of the outer corners of the case shell without a handle.
Aspect 11: The emergency case according to Aspect 9, wherein a stacking centering apparatus is formed at the end of the handle and wherein a stacking centering apparatus is formed on each of the cover caps.
Aspect 12: The emergency case according to any one of Aspects 9 to 11, in which the two stacking centering apparatuses (30a, 30b, 44a, 44b) of a case shell (10) are complementary to one another.
Aspect 13: The emergency case according to any one of Aspects 1 to 12, in which each case shell is part of an emergency case half, which is detachably connected to the other emergency case half.
Aspect 14: The emergency case according to any one of Aspects 1 to 13, in which each case shell has an inner lid.
Aspect 15: The emergency case according to Aspect 14, comprising stacking centering apparatuses on each inner lid.
Aspect 16: The emergency case according to any one of Aspects 1 to 15, comprising walls that are configured to be at least partially fluorescent and/or printed to be fluorescent and/or provided with fluorescent stickers, wherein the fluorescence is in particular photoluminescence, in particular with upconversion and/or downconversion.
Aspect 17: The emergency case according to any one of Aspects 1 to 16, comprising a handle that is configured to be at least partially fluorescent and/or printed to be fluorescent and/or provided with fluorescent stickers, wherein the fluorescence is in particular photoluminescence, in particular with upconversion and/or downconversion.
Aspect 18: The emergency case having the features both of Aspect 16 and Aspect 17, in which the handle fluoresces more brightly than the walls.
Aspect 19: The emergency case according to any one of Aspects 1 to 18, filled with at least one of: plasters and dressings; disposable gloves; first aid scissors and tweezers; hygienic face masks; hygienic clothing; emergency showers and eyewash kits; disinfectants and dispensers therefor; defibrillators and accessories; respiratory masks.
Aspect 20: Use of a case comprising at least one case shell, wherein a handle is formed on the case shell, which handle, in an upright position of the emergency case, is arranged on an upper outer corner of the case shell in each case at two shoulder ends of the handle, extends vertically upward in a first portion, and extends toward the inner longitudinal edge of the case shell remote from the corners in a second portion, and wherein a third portion of the handle connecting the two second portions extends along the inner longitudinal edge, as an emergency case.
Aspect 21: The use of the emergency case according to any one of Aspects 1 to 19 by a person providing medical treatment in a medical emergency situation, wherein, according to the use, proceeding from the situation whereby the emergency case is lying with an outer face of the case shell on the ground or a surface, the person providing treatment grasps the third portion of said emergency case with their hand and pulls said emergency case toward them

An embodiment that has at least one of the following aspects is as well particularly advantageous:
1. An emergency case (100, 200) comprising two emergency case halves, which each comprise a case shell (10, 10′) and an inner lid (26), wherein the emergency case halves are detachably connected, and wherein each inner lid can be locked to its case shell (10, 10′) to produce a closed emergency case half.
2. The emergency case (100, 200) according to Aspect 1, characterized in that the emergency case halves are detachably connected solely by means (50, 52) locat-ed on the case shells (10, 10′).
3. The emergency case (100, 200) according to Aspect 1 or 2, comprising identically constructed case shells (10, 10′).
4. The emergency case (100, 100′) according to Aspect 3, characterized in that a rod (50) and, coaxially therewith and so as to be reversed, a connection device (52) that is in particular C-shaped in cross section for the rod (50) of the other case shell (10, 10′) are arranged on each case shell (10, 10′).
5. The emergency case (100, 200) according to Aspect 4, characterized in that coaxi-ally extending hinges (54a, 54b, 54c) for the respective inner lid of the case shell (10, 10′) are arranged between the rod (50) and the connection device (52), wherein the hinges (54a, 54b, 54c) of one case shell (10′) engage in gaps (56a, 56b, 56c) between the hinges (54a, 54b, 54c) for the other case shell (10).
6. The emergency case (100, 200) according to any of the preceding Aspects, where-in at least one of the emergency case halves comprises a handle (12) on the case shell (10).
7. The emergency case (100) according to Aspect 6, characterized in that both emergency case halves comprise a handle (12).
8. The emergency case (200) according to Aspect 6, characterized in that only one emergency case half (10) comprises a handle (12) and the case shell of the other emergency case half comprises two cover caps (42a, 42b).
9. The emergency case (100, 200) according to any of Aspects 6 to 8, characterized in that each handle (12), proceeding from two shoulder ends each on an upper outer corner (14a, 14b) of the respective case shell (10), extends vertically up-ward in a first portion (16a, 16b), extends toward the inner longitudinal edge (22) of the respective case shell (10) in a second portion (18a, 18b) following each first portion, and wherein a third portion (20) connects the two second portions (18a, 18b).
10. The emergency case (100, 200), comprising stacking centering apparatuses (30a, 30b, 32a, 32b, 34a, 34b, 36a, 36b, 38a, 38b, 40a, 40b and 44a, 44b).
11. The emergency case (100, 200) according to any one of the preceding Aspects, characterized in that the inner lid is or can be latched to the associated case shell (10, 10′).
12. The emergency case (100, 200) according to any one of the preceding Aspects, characterized in that each inner lid (26) can be opened by 90°.
13. The emergency case (100, 200) according to any one of the preceding Aspects, characterized in that when the case is open, an inner lid is rotatable by 180°, such that it comes to rest on the other inner lid.
14. The emergency case (100, 200) according to Aspect 12 or 13, characterized in that the inner lids (26) latch to one another when both inner lids are opened by 90° or one inner lid is rotated by 180°.
15. The emergency case (100, 200) according to any of the preceding Aspects, which is filled with at least one of: plasters and dressings, disposable gloves, first aid scissors and tweezers, hygienic face masks, hygienic clothing, emergency showers and eyewash kits, disinfectants and dispensers therefor, defibrillators and accessories, respiratory masks.
16. An emergency case half, comprising a case shell (10) and an inner lid (26), wherein the case shell (10) can be connected to an identically constructed case shell (10, 10′), in particular can be connected without tools.
17. The emergency case half according to Aspect 16, which is filled with at least one of: plasters and dressings, disposable gloves, first aid scissors and tweezers, hygienic face masks, hygienic clothing, emergency showers and eyewash kits, disinfectants and dispensers therefor, defibrillators and accessories, respiratory masks.
18. The emergency case half according to Aspect 16 or 17, on the case shell of which a rod (50) and, coaxially therewith and so as to be reversed, a connection device (52) that is in particular C-shaped in cross section for the rod (50) of an identically constructed case shell are arranged.
19. The emergency case half according to any one of Aspects 16 to 18, in which the inner lid (26) is opaque.
20. A method for connecting two emergency case halves according to Aspect 18, in which the two rods (50) of the emergency case halves are inserted into the con-nection device (52) of the respectively other emergency case half.
21. Use of the emergency case half according to any one of Aspects 16 to 18 on its own as an emergency case that is separately portable.

An embodiment that has at least one of the following aspects is as well particularly advantageous:
1. first-aid case (100), characterized in that at least one partial area of the first-aid case (100) contains a phosphorescent material.
2. first-aid case (100) according to one of the preceding Aspects, characterized in that the first-aid case (100) has two case shells (10) and the partial regions which contain the phosphorescent material are attachments which can be attached to the case shells (10).
3. first-aid case (100) according to Aspect 2, characterized in that the attachable add-on parts are selected from at least one of the following elements: handle (20), component of the handle (20), in particular insert part (30), fastener (40), component of the fastener (40), in particular actuating element, stack centering device (50), decorative strip.
4. first-aid case (100) according to one of Aspects 2 or 3, characterized in that the handle (20) and/or a case shell (10) contains or contain phosphorescent material.
5. first-aid case (100) according to one of the preceding Aspects, characterized in that the phosphorescent material has an afterglow duration of at least 1 h, preferably at least 2 h, particularly preferably at least 3 h.
6. first-aid case (100) according to one of the preceding Aspects, characterized in that the mass fraction of the phosphorescent material in the partial regions or the case shell (10) is at least 10%, preferably at least 30%.
7. first-aid kit (100) according to one of the preceding Aspects, characterized in that the phosphorescent material is a pigment containing particles of SrAl2O4:Eu, Dy, which preferably have mean diameters d50=25±4 μm and d90≤60 μm.
8. first-aid kit (100) according to one of the preceding Aspects, characterized in that the first-aid kit (100) is filled with at least one of: plasters and dressing material, disposable gloves, first aid scissors and tweezers, hygienic face mask, hygienic clothing, emergency showers and eyewash, disinfectants and dispensers, defibrillators and accessories, respiratory masks.
9. first-aid kit (100) according to one of the preceding Aspects, characterized in that at least partial surface regions (60) are metallized.
10. a method of manufacturing a first aid kit (100) comprising
providing two case shells (10) and a handle (20);
providing add-on parts which can be attached to the case shells (10) and/or the handle (20) and which contain a phosphorescent material;
connecting the two case shells (10) to form a first aid case (100);
attaching the handle (20) to a case shell (10);
attaching the attachable attachments to the case shells (10) and/or the handle (20).
11. method according to Aspect 10, wherein
the case shells (10) and the handle (20) are produced from a thermoplastic material, preferably from an acrylonitrile-butadiene-styrene plastic, in a first injection molding process;
the attachable attachment parts are produced in a second injection molding process from a thermoplastic material, preferably from an acrylonitrile-butadiene-styrene plastic, the phosphorescent material being added to the plastic prior to the injection molding process.
12. process according to Aspect 11, wherein phosphorescent material is added to the thermoplastic for the production of the case shells (10) and/or the handle (20) before the first injection molding process.
13. method according to one of Aspects 10 to 12 with the additional step of metallizing at least partial surface areas (60) of the case shells (10) and/or the handle (20).
14. method according to Aspect 13, wherein the metallization is realized by the following steps:
Etching the surface portion regions (60) of the first aid kit (100) with a solution consisting of chromic acid, sulfuric acid and water, wherein the etching is carried out at a temperature of at least 60° C., preferably at 70° C.;
activating the partial surface regions (60) with palladium, the palladium being colloidally dissolved and surrounded by a protective colloidal shell or being ionically present;
wetting the surface subregions (60) with an accelerator solution, the accelerator solution being selected such that it reduces ionically present palladium or, in the case of colloidally dissolved palladium, removes the protective colloidal shell surrounding the colloidal palladium;
Chemical deposition of nickel on the surface sub-areas (60);
Electroplating of copper on the nickel-plated surface sub-areas (60);
Electroplating of nickel on the copper-plated surface sub-areas (60);
Electroplating of chromium on the nickel-plated surface sections (60);
drying the surface portion regions (60), preferably at a temperature of 67° C.,
   wherein between the individual steps the partial surface regions (60) are rinsed with water, preferably with deionized water.

Although the invention has been described with reference to specific embodiments, it should go without saying that the invention is not limited to these examples and that numerous modifications can be intended and conceived by a person skilled in the art once they have considered the examples. The invention can therefore also be described and implemented without limitation and by way of example on the basis of the following embodiments. The following embodiments can be preferred embodiments. Accordingly, the term “clause” as used herein denotes such a “preferred embodiment”.
Clause 1: An emergency case comprising at least one case shell, wherein a handle is formed on the case shell, which handle, in an upright position of the emergency case, is arranged on an upper outer corner of the case shell in each case at two shoulder ends of the handle, extends vertically upward in a first portion, and extends toward the inner longitudinal edge of the case shell remote from the corners in a second portion, and wherein a third portion of the handle connecting the two second portions extends along the inner longitudinal edge.
Clause 2: The emergency case according to clause 1, in which the transition between the first portion and the second portion and/or the transition between the second portion and the third portion is rounded.
Clause 3: The emergency case according to clause 1 or 2, in which the second portion of the handle extends partially in parallel with an outer longitudinal edge of the case shell and then extends further toward the inner longitudinal edge in a straight line.
Clause 4: The emergency case according to any one of clauses 1 to 3, in which the third portion extends centrally along the inner longitudinal edge.
Clause 5: The emergency case according to clause 4, in which each handle is formed to be symmetrical.
Clause 6: The emergency case according to any one of clauses 1 to 5, comprising two case shells that are complementary to one another.
Clause 7: The emergency case according to clause 6, comprising a handle on both case shells.
Clause 8: The emergency case according to clause 7, wherein the third portions of the two handles contact one another.
Clause 9: The emergency case according to clause 7 or 8, wherein a stacking centering apparatus is formed at the end of each handle.
Clause 10: The emergency case according to clause 6, comprising a handle on only one of the two case shells, wherein a cover cap is provided on each of the outer corners of the case shell without a handle.
Clause 11: The emergency case according to clause 9, wherein a stacking centering apparatus is formed at the end of the handle and wherein a stacking centering apparatus is formed on each of the cover caps.
Clause 12: The emergency case according to any one of clauses 9 to 11, in which the two stacking centering apparatuses of a case shell are complementary to one another.
Clause 13: The emergency case according to any one of clauses 1 to 12, in which each case shell is part of an emergency case half, which is detachably connected to the other emergency case half.
Clause 14: The emergency case according to any one of clauses 1 to 13, in which each case shell has an inner lid.
Clause 15: The emergency case according to clause 14, comprising stacking centering apparatuses on each inner lid.
Clause 16: The emergency case according to any one of clauses 1 to 15, comprising walls that are configured to be at least partially fluorescent and/or printed to be fluorescent and/or provided with fluorescent stickers, wherein the fluorescence is in particular photoluminescence, in particular with upconversion and/or downconversion.
Clause 17: The emergency case according to any one of clauses 1 to 16, comprising a handle that is configured to be at least partially fluorescent and/or printed to be fluorescent and/or provided with fluorescent stickers, wherein the fluorescence is in particular photoluminescence, in particular with upconversion and/or downconversion.
Clause 18: The emergency case having the features both of clause 16 and clause 17, in which the handle fluoresces more brightly than the walls.
Clause 19: The emergency case according to any one of clauses 1 to 18, filled with at least one of: plasters and dressings; disposable gloves; first aid scissors and tweezers; hygienic face masks; hygienic clothing; emergency showers and eyewash kits; disinfectants and dispensers therefor; defibrillators and accessories; respiratory masks.
Clause 20: Use of a case comprising at least one case shell, wherein a handle is formed on the case shell, which handle, in an upright position of the emergency case, is arranged on an upper outer corner of the case shell in each case at two shoulder ends of the handle, extends vertically upward in a first portion, and extends toward the inner longitudinal edge of the case shell remote from the corners in a second portion, and wherein a third portion of the handle connecting the two second portions extends along the inner longitudinal edge, as an emergency case.
Clause 21: The use of the emergency case according to any one of clauses 1 to 19 by a person providing medical treatment in an emergency medical situation, wherein, according to the use, proceeding from the situation whereby the emergency case is lying with an outer face of the case shell on the ground or a surface, the person providing treatment grasps the third portion of said emergency case with their hand and pulls said emergency case toward them.

The invention can also be described without limitation and by way of example by the following further clauses:
Clause 1: An emergency case comprising two emergency case halves,
which each comprise a case shell and an inner lid, wherein the emergency case halves are detachably connected, and wherein each inner lid can be locked to its case shell to produce a closed emergency case half.
Clause 2: The emergency case according to clause 1, characterized in that the emergency case halves are detachably connected solely by means located on the case shells.
Clause 3: The emergency case according to clause 1 or 2, comprising identically constructed case shells.
Clause 4: The emergency case according to clause 3, characterized in that a rod and, coaxially therewith and so as to be reversed, a connection device that is in particular C-shaped in cross section for the rod of the other case shell are arranged on each case shell.
Clause 5: The emergency case according to clause 4, characterized in that coaxially extending hinges for the respective inner lid of the case shell are arranged between the rod and the connection device, wherein the hinges of one case shell engage in gaps between the hinges for the other case shell.
Clause 6: The emergency case according to any one of the preceding clauses, wherein at least one of the emergency case halves comprises a handle on the case shell.
Clause 7: The emergency case according to clause 6, characterized in that both emergency case halves comprise a handle.
Clause 8: The emergency case according to clause 6, characterized in that only one emergency case half comprises a handle and the case shell of the other emergency case half comprises two cover caps.
Clause 9: The emergency case according to any one of clauses 6 to 8, characterized in that each handle, proceeding from two shoulder ends each on an upper outer corner of the respective case shell, extends vertically upward in a first portion, extends toward the inner longitudinal edge of the respective case shell in a second portion following each first portion, and wherein a third portion connects the two second portions.
Clause 10: The emergency case, comprising stacking centering apparatuses.
Clause 11: The emergency case according to any one of the preceding clauses, characterized in that the inner lid is or can be latched to the associated case shell.
Clause 12: The emergency case according to any one of the preceding clauses, characterized in that each inner lid can be opened by 90°.
Clause 13: The emergency case according to any one of the preceding clauses, characterized in that when the case is open, an inner lid is rotatable by 180°, such that it comes to rest on the other inner lid.
Clause 14: The emergency case according to clause 12 or 13, characterized in that the inner lids latch to one another when both inner lids are opened by 90° or one inner lid is rotated by 180°.
Clause 15: The emergency case according to any one of the preceding clauses, which is filled with at least one of: plasters and dressings, disposable gloves, first aid scissors and tweezers, hygienic face masks, hygienic clothing, emergency showers and eyewash kits, disinfectants and dispensers therefor, defibrillators and accessories, respiratory masks.
Clause 16: An emergency case half, comprising a case shell and an inner lid, wherein the case shell can be connected to an identically constructed case shell, in particular can be connected without tools.
Clause 17: The emergency case half according to clause 16, which is filled with at least one of: plasters and dressings, disposable gloves, first aid scissors and tweezers, hygienic face masks, hygienic clothing, emergency showers and eyewash kits, disinfectants and dispensers therefor, defibrillators and accessories, respiratory masks.
Clause 18: The emergency case half according to clause 16 or 17, on the case shell of which a rod and, coaxially therewith and so as to be reversed, a connection device that is in particular C-shaped in cross section for the rod of an identically constructed case shell are arranged.
Clause 19: The emergency case half according to any one of clauses 16 to 18, in which the inner lid is opaque.
Clause 20: A method for connecting two emergency case halves according to clause 18, in which the two rods of the emergency case halves are inserted into the connection device of the respectively other emergency case half.
Clause 21: Use of the emergency case half according to any one of clauses 16 to 18 on its own as an emergency case that is separately portable.

An embodiment that has at least one of the following aspects is particularly advantageous:
1. An emergency case (100, 200) comprising two emergency case halves, which each comprise a case shell (10, 10′), wherein partition elements (300a, 300b, 300c, 300d) for forming compartments are provided in at least one case shell (10, 10′), wherein a series of connection apparatuses (66) is provided in each case directly as part of at least two inner walls of this case shell and wherein the partition elements (300a, 300b, 300c, 300d) are inserted at least in one part of the connection apparatuses.
2. The emergency case according to Aspect 1, comprising rectangular case shells (10, 10′) and partition elements (300a, 300b, 300c, 300d), which extend in a straight line and are each inserted in opposite connection apparatuses (66) of a case shell (10, 10′).
3. The emergency case according to Aspect 1 or 2, in which a reinforcement rib (68) on the outer wall corresponds in position to at least one and preferably each connection apparatus (66) on the inner wall of the case shell (10, 10′).
4. The emergency case according to any one of Aspects 1 to 3, in which each case shell (10, 10′) and/or each partition element is produced in one piece from hard plastics material.
5. The emergency case according to any one of Aspects 1 to 4, in which the partition elements (300a, 300b, 300c, 300d) comprise a strip-shaped main body (310a, 310b, 310c, 310d) and in which a series of connection apparatuses (320-1, 320-2) for other partition elements are provided on at least one side of the strip-shaped main body (310a, 310b, 310c, 310d).
6. The emergency case according to Aspect 5, in which the connection apparatuses (320-1, 320-2) of the partition elements (300a, 300b, 300c, 300d) each comprise a plurality of lugs (322), which are arranged to alternate with one another to the right and the left.
7. The emergency case according to any one of Aspects 1 to 6, in which the partition elements (300a, 300b, 300c, 300d) comprise a sliding track (330), which can be inserted into a connection apparatus, on at least one longitudinal end.
8. The emergency case according to any one of Aspects 1 to 7, which is filled with at least one of: plasters and dressings; disposable gloves; first aid scissors and tweezers; hygienic face masks; hygienic clothing; emergency showers and eyewash kits; disinfectants and dispensers therefor; defibrillators and accessories; respiratory masks.
9. The emergency case (100, 200) according to any one of Aspects 1 to 8, in which partition elements (300a, 300b, 300c, 300d) for forming compartments are provided in each case shell (10, 10′).
10. An emergency case half comprising a case shell (10, 10′) and a series of connection apparatuses (66) each arranged directly as part of at least two inner walls of the case shell (10, 10′), wherein partition elements (300a, 300b, 300c, 300d) for forming compartments are inserted in at least one part of the connection apparatuses (66).
11. The emergency case half according to Aspect 10, comprising an inner lid (26), wherein the inner lid (26) can be locked to the case shell (10, 10′) to produce a closed emergency case half.
12. The emergency case half according to Aspect 10 or 11, which is filled with at least one of: plasters and dressings; disposable gloves; first aid scissors and tweezers; hygienic face masks; hygienic clothing; emergency showers and eyewash kits; disinfectants and dispensers therefor; defibrillators and accessories; respiratory masks.
13. A system of emergency cases (100a, 100b, 100c) having the features according to any one of Aspects 1 to 9 of different sizes and/or of emergency case halves having the features according to any one of Aspects 10 to 12 of different sizes, wherein the dimensions of the case shells (10, 10′) are coordinated with the connection apparatuses (66) among themselves and with the dimensions of the partition elements (300a, 300b, 300c, 300d) such that predetermined partition elements (300a, 300b, 300c, 300d) can be used in a plurality of emergency cases (100a, 100b, 100c) and/or emergency case halves having case shells (10, 10′) of different sizes.
14. The system according to Aspect 13, in which the predetermined partition elements (300a, 300b, 300c, 300d) form a group as a kit which can be used in all the emergency cases (100a, 100b, 100c) or emergency case halves of the system.
15. A partition element (300a, 300b, 300c, 300d) for a case shell (10, 10′) of an emergency case (100, 200) and comprising a strip-shaped main body (310a, 310b, 310c, 310d), on which connection apparatuses (320-1, 320-2) are formed, wherein each connection apparatus (320-1, 320-2) comprises lugs (322), which are arranged to alternate with one another to the right and the left.
16. The partition element according to Aspect 15, in which connection apparatuses (320-1, 320-2) are provided on each side of the strip-shaped main body (310a, 310b, 310c, 310d), wherein a cut-out (324) on one side, to which a lug on the other side corresponds, is opposite each lug (322) on this one side, and wherein the corresponding cut-out on the other side, which is opposite the lug on the other side, corresponds to the lug (322) on this one side.

An embodiment that has at least one of the following aspects is particularly advantageous:
1. A system comprising a wall bracket and an emergency case, wherein retaining arms of the wall bracket are formed to match to a handle of the emergency case such that the handle rests on each of the retaining arms by a lateral portion, and at the same time can be grasped by a central portion between the retaining arms in order to take the emergency case down from the wall bracket.
2. The system according to Aspect 1, wherein the wall bracket comprises exactly two retaining arms.
3. The system according to Aspects 1 or 2, wherein the emergency case comprises at least one case shell, wherein the handle is formed on the case shell, wherein when the emergency case is in an upright position, the handle is arranged on upper outer corners of the case shell at two shoulder ends of the handle, the handle extending vertically upward in two first portions, and extending toward an inner longitudinal edge of the case shell remote from the corners in two second portions, and wherein a third portion of the handle connecting the two second portions extends along the inner longitudinal edge, such that the emergency case can rest on the retaining arms by the second portions and at the same time can be grasped by the third portion.
4. The system according to Aspects 1 or 2, wherein the emergency case comprises two emergency case halves, wherein when one emergency case half is hanging on the wall bracket, the other emergency case half can be opened up, and wherein the wall bracket comprises a stop for the opened-up emergency case half in a foot region.
5. The system according to Aspect 4, wherein the stop is configured such that the other emergency case half can be opened at an angle of between 70° and 110°.
6. The system according to Aspect 4, wherein the two emergency case halves are configured to be detached from one another when the emergency case is opened up until it reaches the stop.
7. The system according to Aspect 4, wherein the emergency case comprises ribs and the stop has molded portions that are complementary to the ribs.
8. The system according to Aspects 1 or 2, comprising a fastener device on a retaining arm of the retaining arms.
9. The system according to Aspect 8, wherein the fastener comprises a pin that engages in a cut-out in the emergency case, the pin configured to be pulled out of the cut-out by a pull tab.
10. The system according to Aspect 9, wherein the pull tab is pulled counter to a force of a spring.
11. The system according to Aspects 1 or 2, wherein the emergency case is filled with one of: plasters and dressings; disposable gloves; first aid scissors and tweezers; hygienic face masks; hygienic clothing; emergency showers and eyewash kits; disinfectants and dispensers therefor; defibrillators and accessories; respiratory masks.
12. A wall bracket for an emergency case, comprising a rear bar and at least two upper retaining arms for receiving an emergency case handle and comprising a stop in a foot region for supporting the emergency case in an opened-up state.
13. The wall bracket according to Aspect 12, comprising exactly two retaining arms.
14. The wall bracket according to Aspects 12 or 13, wherein the retaining arms each comprise a seat for a portion of the emergency case handle and retaining fingers in front of the seat.
15. The wall bracket according to Aspect 14, comprising a fastener device on one of the fingers.
16. The wall bracket according to Aspect 15, wherein the fastener device comprises a pin (474) configured to be pulled forward by a pull tab.
17. The wall bracket according to Aspect 16, wherein the pull tab (472) is pulled counter to a force of a spring.
18. Use of a system according to Aspects 1 or 2, wherein when the emergency case comprises two emergency case halves and is retained on the wall bracket by a rear emergency case half, the use comprises:
grasping the emergency case by the central portion of the handle of at least the rear emergency case half with a hand; and
exerting a force in a vertical direction with the hand such that the emergency case is taken down from the wall bracket.
19. Use of an emergency case half of the emergency case in the system according to Aspect 6, wherein when the emergency case is retained on the wall bracket by a rear emergency case half, the use comprises:
first opening up a front emergency case half until it is retained by the stop; and
then detaching the front emergency case half from the rear emergency case half.
20. Use of an emergency case half of the emergency case in the system according to Aspect 10, wherein when the emergency case comprising two emergency case halves is retained on the wall bracket by a rear emergency case half, the use comprises:
pulling the pull tab with one hand such that the pin is disengaged with the cut-out in the emergency case; and
exerting a force on the emergency case in a vertical direction with the other hand such that the emergency case is taken down from the wall bracket.

An embodiment that has at least one of the following aspects is particularly advantageous:
1. An emergency case (100, 200) comprising a handle (12) for carrying the emergency case (100, 200), wherein the handle (12) comprises a main body (70, 72), wherein the handle (12) comprises a cavity (76) for receiving an electronics module (86).
2. The emergency case (100, 200) according to Aspect 1, in which the main body of the handle (12) is formed as a plastics injection-molded part which forms a hollow profile in which the cavity (76) is located.
3. The emergency case (100, 200) according to Aspect 1 or 2, in which the main body of the handle (12) is open to one side.
4. The emergency case (100, 200) according to any one of Aspects 1 to 3, in which a closure cap (74) is or can be inserted into the main body of the handle (12).
5. The emergency case (100, 200) according to Aspect 4, in which the closure cap (74) comprises a strip-shaped main body (80).
6. The emergency case according to any one of Aspects 4 or 5, in which the closure cap (74) can be pulled out in order to make it possible to insert or remove an electronics module (86).
7. The emergency case according to any one of Aspects 4 to 6, in which the closure cap (74) can be locked by a snap-in device.
8. The emergency case according to Aspect 7, in which studs (82) of the closure cap (74) are inserted into circular cut-outs (78).
9. The emergency case (100, 200) according to any one of Aspects 1 to 8, comprising an electronics module (86) in the handle (12).
10. The emergency case (100, 200) according to Aspect 9, in which the electronics module (86) comprises at least one of:
a position sensor for detecting the position of the electronics module (86) and therefore the emergency case (100, 200);
a transmission apparatus for outputting signals for making it easier to find the electronics module (86) and therefore the emergency case (100, 200);
a receiving module for receiving a query signal.
11. The emergency case (100, 200) according to Aspect 9 or 10, in which the electronics module (86) is configured to draw energy from query signals in order to use it for sending a response signal.
12. The emergency case (100, 200) according to any one of Aspects 9 to 11, in which the electronics module (86) comprises a memory in which information regarding the target contents of the case is stored, wherein the electronics module (86) is configured to output this information via a communication interface.
13. The emergency case according to any one of Aspects 1 to 12, comprising a case shell (10, 10′) on which the handle (12) is formed, which, in an upright position of the emergency case (100, 200), is arranged on an upper outer corner of the case shell in each case at two shoulder ends of the handle (12), extends vertically upward in a first portion (16a, 16b), and extends toward the inner longitudinal edge (22) of the case shell (10) remote from the corners in a second portion (18a, 18b), and wherein a third portion (20) of the handle (12) connecting the two second portions (18a, 18b) extends along the inner longitudinal edge (22).
14. The emergency case according to Aspect 13, in which a receptacle in the third portion (20) of the handle (12) for an electronics module (86), preferably in the region of an insert tab (84) of a or the closure cap (74), is present.
15. The emergency case according to Aspect 13 or 14, in which:
the transition between the first portion and the second portion and/or the transition between the second portion and the third portion is rounded, and/or
the second portion (18) of the handle (12) extends partially (181a, 181b) in parallel with an outer longitudinal edge of the case shell (10) and then extends (182a, 182b) further toward the inner longitudinal edge in a straight line, and/or
the third portion (20) extends centrally along the inner longitudinal edge, and/or
each handle (12) is formed to be symmetrical.
16. The emergency case (100, 200) according to any one of Aspects 1 to 15, comprising two case shells (10, 10′) that are complementary to one another.
17. The emergency case (100) according to Aspect 16, comprising a handle (12) on both case shells (10).
18. The emergency case according to Aspect 17, comprising two emergency case halves, which each comprise a case shell (10), a handle (12), and an inner lid (26), wherein the emergency case halves are detachably connected, and wherein each inner lid (26) can be locked to its case shell (10) to produce a closed emergency case half, and wherein each emergency case half comprises an electronics module (86) in the respective handle (12).
19. The emergency case according to Aspect 16, comprising a handle (12) on only one of the two case shells (10).
20. The emergency case according to any one of Aspects 1 to 19, filled with at least one of: plasters and dressings; disposable gloves; first aid scissors and tweezers; hygienic face masks; hygienic clothing; emergency showers and eyewash kits; disinfectants and dispensers therefor; defibrillators and accessories; respiratory masks.
21. An emergency case half comprising a handle (12) for carrying the emergency case (100, 200), wherein the handle (12) comprises a main body (70, 72), wherein the handle (12) comprises a cavity (76) for receiving an electronics module (86).
22. Use of an emergency case (100, 200) having the features according to Aspect 6, comprising the steps of:
removing the closure cap (74) from the main body of the handle (12);
inserting an electronics module (86) into the cavity (76) in the handle (12);
closing the handle again by inserting the closure cap (74) into the main body of the handle (12).
23. The use according to Aspect 22 in an emergency case (100, 200) having the features according to any one of Aspects 9 to 12, in which, before an electronics module (86) is inserted into the cavity (76), the previous electronics module (86) is removed from the cavity (76).

An embodiment that has at least one of the following aspects is particularly advantageous:
1. An emergency case (100, 100′) comprising two case shells (10, 10′) and a seal (20, 20′), characterized in that the seal (20, 20′) is formed by a sealing material, wherein the sealing material is materially bonded to one of the case shells (10, 10′), and wherein the seal (20, 20′) is in contact with both case shells (10, 10′) in the closed state of the emergency case (100, 100′).
2. The emergency case (100, 100′) according to Aspect 1, characterized in that the sealing material is a flexible foam.
3. The emergency case (100, 100′) according to Aspect 2, characterized in that the flexible foam is a polyurethane foam which is crosslinked at room temperature and results from a reaction of a polyol or a mixture of polyols and methylene diphenyl diisocyanate or one of its derivatives.
4. The emergency case (100, 100′) according to any one of the preceding Aspects, characterized in that the case shells (10, 10′) consist of acrylonitrile butadiene styrene plastics material.
5. The emergency case (100, 100′) according to any one of the preceding Aspects, comprising a pressing element (30), wherein the pressing element (30) is formed on or fastened to a case shell (10, 10′) opposite the seal (20), and wherein the pressing element (30) is configured and positioned such that it exerts mechanical pressure on the seal (20, 20′) in the closed state of the emergency case (100, 100′).
6. The emergency case (100, 100′) according to any one of the preceding Aspects, characterized in that one of the case shells (10, 10′) comprises a receiving groove (40, 40′) running along its edge and the seal (20, 20′) is arranged in this receiving groove (40, 40′).
7. The emergency case (100, 100′) according to Aspect 6, characterized in that the seal (20, 20′) projects beyond the peripheral receiving groove (40, 40′).
8. The emergency case (100, 100′) according to any one of the preceding Aspects, characterized in that one of the case shells (10, 10′) comprises partition elements (50) for forming sub-sections which can be inserted into the case shell (10, 10′), and wherein additional seals (20, 20′) are arranged on the case shell (10, 10′) opposite the insertable partition elements (50) and correspond, at least in one direction, to the contour of the sub-sections that can be formed by the insertable partition elements (50), wherein the additional seals (20, 20′) are in contact with longitudinal edges of the insertable partition elements (50) in the closed state of the emergency case (100, 100′).
9. The emergency case (100, 100′) according to Aspect 8, characterized in that the additional seals (20, 20′) are arranged such that they correspond to the contours of the sub-sections that can be formed by the insertable partition elements (50), wherein the additional seals (20, 20′) are in contact with longitudinal edges of the insertable partition elements (50) in the closed state of the emergency case (100, 100′).
10. The emergency case (100, 100′) according to any one of Aspects 1 to 5 or 8 to 9, characterized in that fixed partition elements (60) for forming sub-sections are formed on one of the case shells (10, 10′), wherein the seal (20, 20′) is arranged on a longitudinal edge of one of the fixed partition elements (60), wherein the longitudinal edge preferably comprises a receiving groove (40, 40′) and the seal (20, 20′) is arranged in this receiving groove.
11. The emergency case (100, 100′) according to any one of Aspects 1 to 7, characterized in that fixed partition elements (60) for forming fixed sub-sections are formed on one of the case shells (10, 10′), wherein an additional seal (20, 20′) is arranged on a longitudinal edge of one of the fixed partition elements (60) and is materially bonded to the longitudinal edge, wherein the longitudinal edge preferably comprises a receiving groove (40, 40′) and the seal (20, 20′) is arranged in this receiving groove (40, 40′).
12. The emergency case (100, 100′) according to any one of the preceding Aspects, characterized in that the emergency case (100, 100′) is filled with at least one of: plasters and dressings, disposable gloves, first aid scissors and tweezers, hygienic face masks, hygienic clothing, emergency showers and eyewash kits, disinfectants and dispensers therefor, defibrillators and accessories, respiratory masks.
13. A double emergency case comprising two emergency cases (100, 100′) according to any one of the preceding Aspects, characterized in that the two emergency cases (100, 100′) are detachably connected to one another, wherein a case shell (10, 10′) for forming a closed emergency case (100, 100′) can be locked to the corresponding case shell (10, 10′) of the same emergency case (100, 100′) in each case.
14. A method for producing an emergency case (100, 100′), which comprises:
providing two case shells (10, 10′);
applying a flowable sealing component to a case shell (10, 10′), wherein the flowable sealing component is selected such that, after being applied, it both forms a chemical bond with the surface of the case shell (10, 10′) and reacts to form a solid sealing material, by means of which the resulting sealing material is materially bonded to the case shell (10, 10′) and forms a seal (20, 20′);
connecting the two case shells (10, 10′) to form an emergency case (100, 100′) in such a way that the seal (20, 20′) is in contact with both case shells (10, 10′) in the closed state of the emergency case (100, 100′).
15. The method according to Aspect 14, wherein one of the case shells (10, 10′) comprises a receiving groove (40, 40′) running along the edge and wherein the flowable sealing component is applied within the receiving groove (40, 40′).
16. The method according to any one of Aspects 14 or 15, wherein the case shells (10, 10′) consist of acrylonitrile butadiene styrene plastics material and the flowable sealing component contains a polyol or a mixture of polyols and methylene diphenyl diisocyanate or one of its derivatives.

### BRIEF DESCRIPTION OF THE DRAWING VIEWS

In the following, the invention will be described in greater detail with reference to the drawings, in which:

FIG. 1 is a perspective view of a first embodiment of the emergency case according to the invention;

FIG. 2 is a front view of the emergency case from FIG. 1;

FIG. 3 is a plan view of the emergency case from FIG. 1;

FIG. 4 is a view from below of the emergency case from FIG. 1;

FIG. 5 is a side view of the emergency case from FIG. 1;

FIG. 6a shows the emergency case from FIG. 1 when it is open;

FIG. 6b further illustrates this when the two inner lids are open;

FIG. 6c further illustrates this when the two inner lids have been folded over to one side;

FIG. 6d shows an emergency case half of the emergency case from FIG. 1 in the open state with partition elements located therein;

FIG. 6e-6h show partition elements of different sizes;

FIG. 6i is an enlarged view of the partition element from FIG. 6f for illustrating the connection apparatuses for the partition elements;

FIG. 6k is a plan view of a series of emergency case halves of different sizes divided into compartments;

FIG. 7a is a perspective view from a first side of a second embodiment of the emergency case according to the invention; and

FIG. 7b shows this from a second side; and

FIG. 8 is a front view of the emergency case from FIG. 7a;

FIG. 9a and FIG. 9b are two different side views of the emergency case from FIG. 7a;

FIG. 10 is a plan view of the emergency case from FIG. 7a; and

FIGS. 11a to 11c show the emergency case from FIG. 7a in views corresponding to FIGS. 6a to 6c;

FIG. 12a is a perspective view from a first side of an emergency case half as an emergency case according to a third embodiment of the invention; and

FIG. 12b shows this from a second side;

FIG. 12c shows the situation in which two emergency case halves are joined together;

FIG. 12d is a schematic cross-sectional view for illustrating the clamping principle used;

FIG. 12e is a perspective view of a case shell without a handle in the closed state;

FIG. 12f is a perspective view of the case shell from FIG. 12e in the open state;

FIG. 13 shows a stacking centering element on the left upper outer corner of the emergency case from FIG. 7;

FIG. 14 shows a stack of a plurality of emergency cases;

FIG. 15a is a detail from FIG. 1 with the handle; and

FIG. 15b is a detail from FIG. 6a with the handle;

FIG. 15c shows the main body of the handle from FIG. 15b on its own, without the insert part;

FIG. 15d shows the insert part (closure cap) that can be inserted into the main body on its own;

FIG. 15e shows the complete handle, on its own, with the main body from FIG. 15c and the insert part from FIG. 15d inserted therein;

FIG. 15f is a section through the emergency case in order to render visible an electronics module located in its handle;

FIG. 15g is an enlarged circular detail from FIG. 15f;

FIG. 16a shows the situation in which a first aider is grasping the case standing on the ground; and

FIG. 16b shows the situation in which a first aider is grasping the case lying on the ground.

FIG. 17 shows a perspective view of a first embodiment of the first aid kit according to the invention;

FIG. 17a shows the case lying on the ground with a handle in the open state of the knob apparatuses;

FIG. 17b is an enlarged circular detail from FIG. 17a;

FIG. 17c is a cross section through a view according to FIG. 17a;

FIG. 17d is a perspective oblique front view of the emergency case with a handle with the knob apparatus closed;

FIG. 17e shows the case from FIG. 17d with the knob apparatus open;

FIG. 17f shows the case from FIG. 17d and FIG. 17e with the knob apparatuses in a stowed position;

FIG. 18 shows a front view of the first-aid kit of FIG. 1;

FIG. 18a is a front view of the wall bracket which can be used with the emergency case in a version without a lock;

FIG. 18b is a side view of the wall bracket from FIG. 18a;

FIG. 18c is a perspective front side view of the wall bracket from FIG. 18a;

FIGS. 18d, 18e, and 18f are views corresponding to FIGS. 18a, 18b, and 18c, respectively, of the same type of wall bracket, but provided with a lock;

FIG. 18g shows the wall bracket without a lock with the emergency case hanging thereon in the closed state;

FIG. 18h shows the wall bracket from FIG. 18g with the emergency case now in the open state;

FIGS. 18i and 18k is a front view and a side view, respectively, of the wall bracket with the open emergency case from FIG. 18h; and

FIGS. 18l, 18m, 18n, and 180 are views corresponding to FIGS. 18g, 18h, 18i, and 18k, respectively, of the same wall bracket, but with a lock.

FIGS. 18p and 18q are separate perspective oblique front and oblique rear views, respectively, of the fastener cap for providing the fastener;

FIG. 18r illustrates the fastener cap from FIGS. 18p and 18q being pulled out on the wall bracket;

FIGS. 18s and 18t are separate perspective oblique front and oblique rear views, respectively, of the cover cap, which can be used instead of the fastener cap;

FIG. 19 shows a front view of a second embodiment of the first aid kit according to the invention;

FIG. 20 is a perspective view of a first embodiment of the first aid case according to the invention;

FIG. 21 is a side view of the first aid case from FIG. 20; and

FIG. 22 is a plan view of the first aid case from FIG. 20; and

FIG. 23 shows the layered structure of a hot stamping foil used when providing the phosphorescent material for the first aid case.

FIG. 24 is a perspective view of a first embodiment of the emergency case according to the invention, with the seal being omitted;

FIG. 24b is a perspective view of the emergency case from FIG. 1 with the seal;

FIG. 24c is a perspective view of the emergency case from FIGS. 1 and 1b in the closed state;

FIG. 25 is a perspective view of a second embodiment of the emergency case comprising variable sub-sections;

FIG. 26 is a perspective view of a third embodiment of the emergency case according to the invention with the seal being omitted;

FIG. 26b is a perspective view of the emergency case from FIG. 3 with the seal;

FIG. 27 is a perspective view of an embodiment of the double emergency case according to the invention with the seal being omitted;

FIG. 27b is a perspective view of the double emergency case from FIG. 4 with the seal;

FIG. 27c is a perspective view of the emergency case from FIGS. 4 and 4b in the closed state; and

FIG. 28 is a perspective view of the application of the flowable sealing component in an embodiment of the method according to the invention.

### DETAILED DESCRIPTION OF THE INVENTION

An emergency case, denoted by 100 as a whole, comprises two case shells 10, which are identical in the present case and, in the case of the emergency case 100, each have identical handles 12. The case shells 10 comprising the handles 12 together form an emergency case, wherein, in the case of the emergency case 100, two emergency case halves can be exactly complementary to one another.

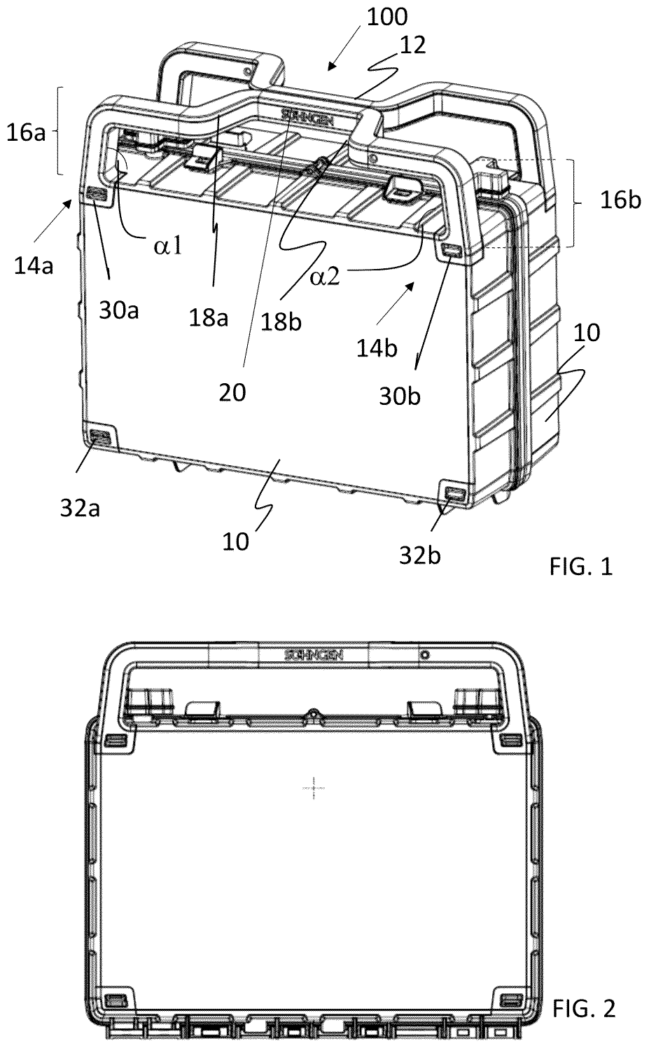

The handles are fastened to the upper outer corners 14a, 14b of each case shell 10, i.e. a first end of the handle 12 is inserted on the first upper outer corner 14a and a second end of the handle 12 is inserted on the other upper outer corner 14b of the case shell 10. In a first portion, the handle 12 extends vertically upward proceeding from the respective upper outer corner; in the present case, these are the portions 16a (first portion on the first handle end) and 16b (first portion on the second handle end) shown in FIG. 1. “Vertically upward” is understood to mean that the tilt angle α1 at the first handle end and the tilt angle α2 at the second handle end are each between 70° and 110°, preferably less than 90°. In the present case, both tilt angles α1 and α2 are preferably identical and have a value of approx. 75° for this purpose, for example.

The plan view according to FIG. 3 shows how the handle 12 extends: in a second portion 18a and 18b, respectively, each handle extends toward the inner longitudinal edge 22 of the case shell. In the present case, the second portion contains two sections, namely the portion 18a contains the portion 181a, which still extends in parallel with the outer longitudinal edge 24 of the case shell 10, and the portion 182a, which extends from the outer longitudinal edge 24 toward the inner longitudinal edge 22. The angle β1 of the tilt of the second section 182a relative to the first section 181a is between 1° and 20°, in the present case approx. 15°. In an analogous manner, the second portion 18b is divided into the sections 181b and 182b, in which the angle β2 is preferably likewise between 1° and 20° and preferably has the same value as the angle β1.

The third portion 20 of the handle 12 extends at the end of the second portion 18b, in particular at the end of its second section 182b in the region of the inner longitudinal edge 22. This central portion extends in particular in parallel with the inner longitudinal edge 22, and in the emergency case 100, the third portion 20 of the first emergency case half on the first case shell 10 and the third portion 20 of the corresponding second emergency case half contact one another immediately above the inner longitudinal edge 22. The length d3 of the third portion 20 is almost a third of the total length of the entire case and therefore each of the portions 18b and 18a make up the other third, or are slightly longer than the third. These dimensions ensure that the case is balanced out when the user grasps the case by the one third portion 20.

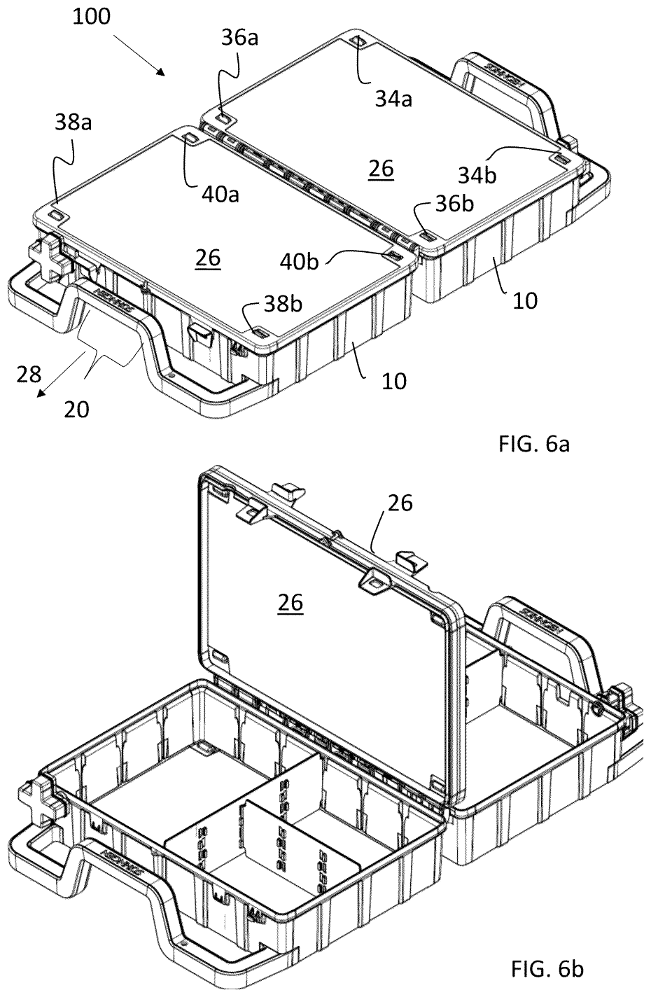

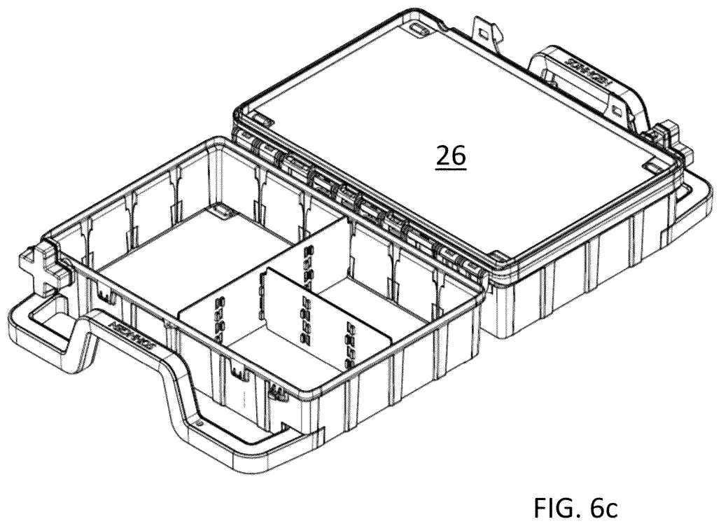

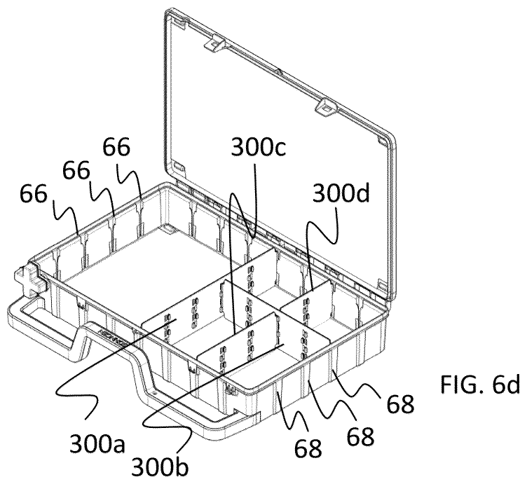

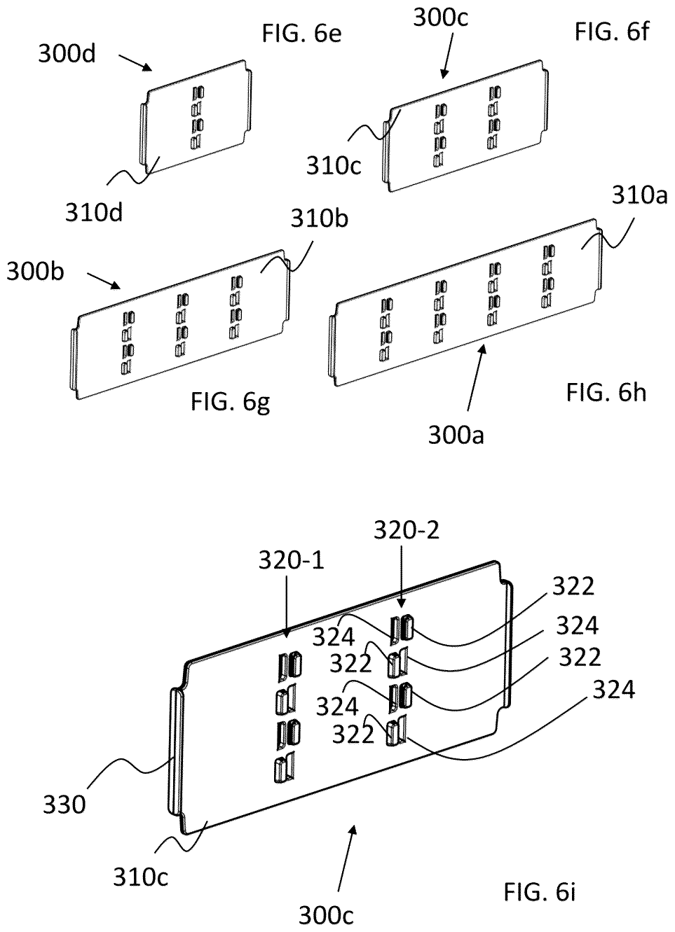

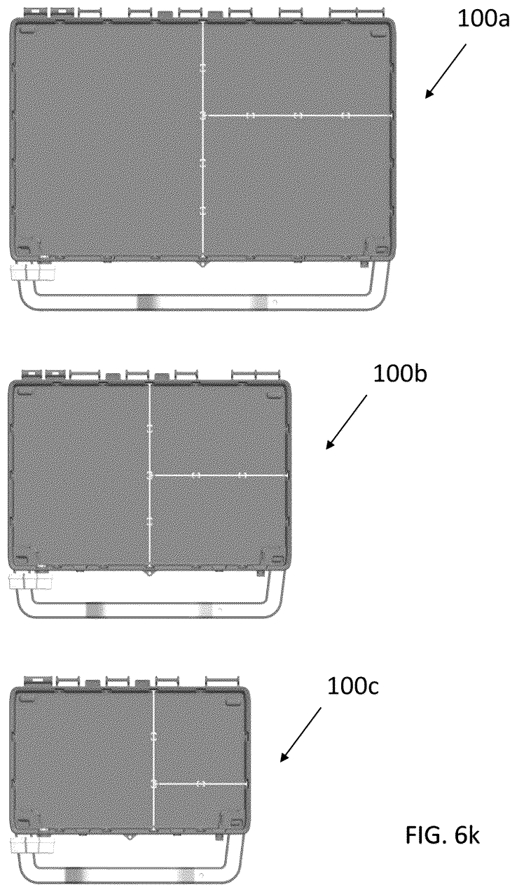

The case can be opened up according to FIG. 6a, which shows that there is a preferably opaque inner lid 26 on the case shell 10. The opened-up emergency case can be optimally grasped by the third portion, which is pointing upward when said case is lying down, such that, for use in an emergency situation on the patient on site, it is possible to rapidly pull over the case, for example in the direction according to the arrow 28 in FIG. 6a.

According to FIG. 6b, the two inner lids 26 can be lifted up and opened up vertically in a manner complementary to one another.

According to FIG. 6c, the two lids that are latched to one another can also be folded to just one side, in the present case the rear side in FIG. 6c.

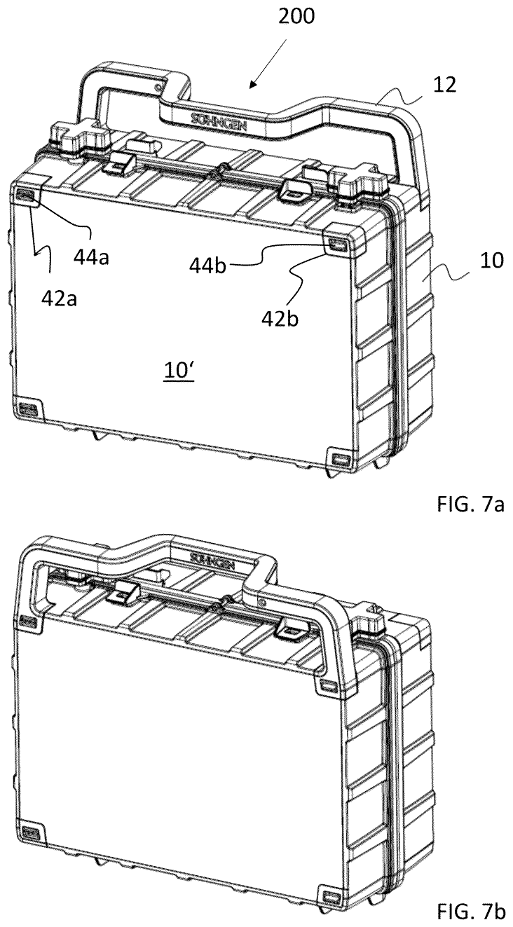

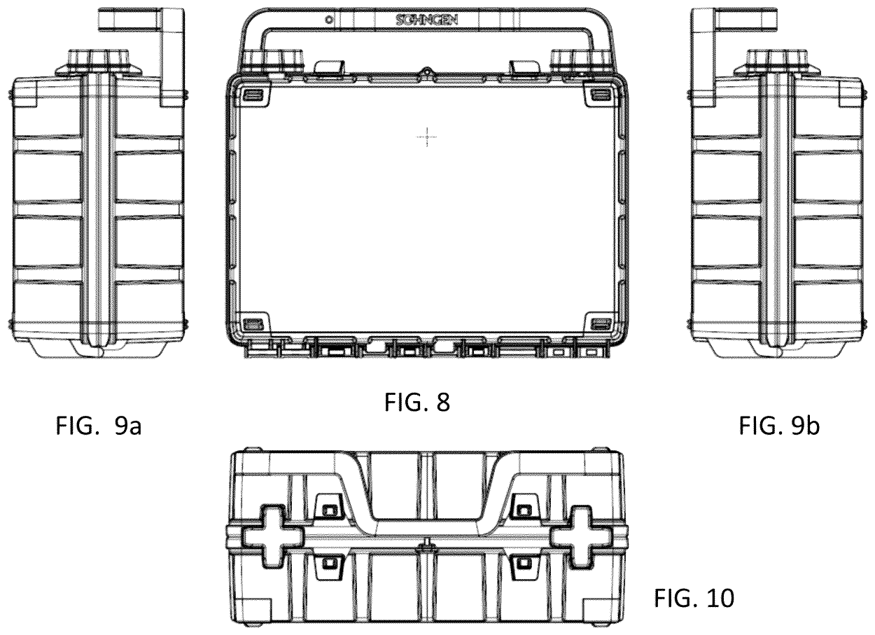

The second embodiment 200 of the emergency case according to FIG. 7a to FIG. 11c, as explained in the following, differs from the above-described embodiment 100 of the emergency case in that the handle 12 is formed on just one case shell 10, while cover caps 42a, 42b are attached to the other case shell 10′ on the upper outer corners. The cover caps replace the handle on the case shell 10′.

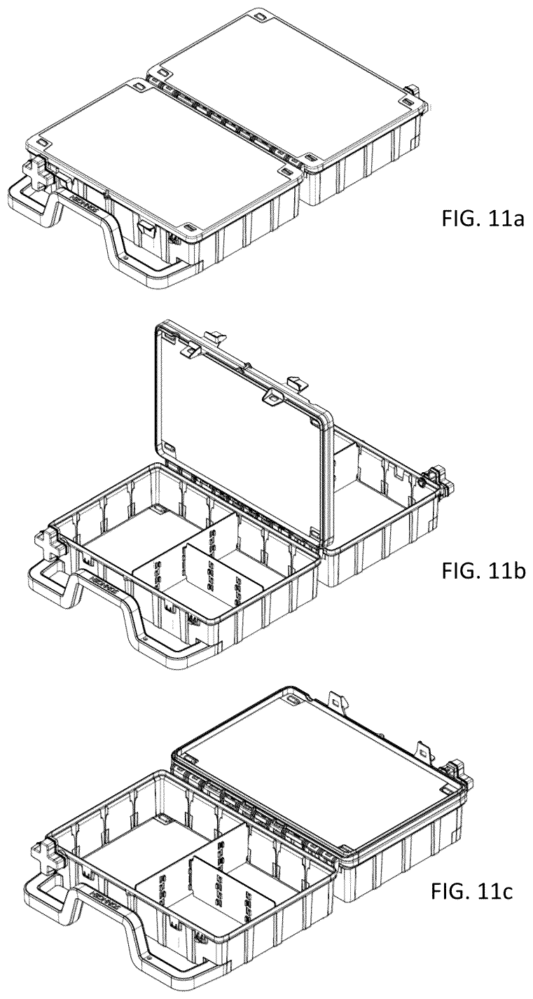

Because a handle is omitted, the emergency case 200 is then lighter than the emergency case 100. Owing to the shape of the handle, its portability is greatly improved, however. FIGS. 11a to 11c correspond to FIGS. 6a to 6c.

So that the emergency cases can be suitably stacked up with other emergency cases (for example in an ambulance), there are so-called stacking centering apparatuses. Apparatuses of this kind are generally complementary to one another in pairs (so as to be reversed).

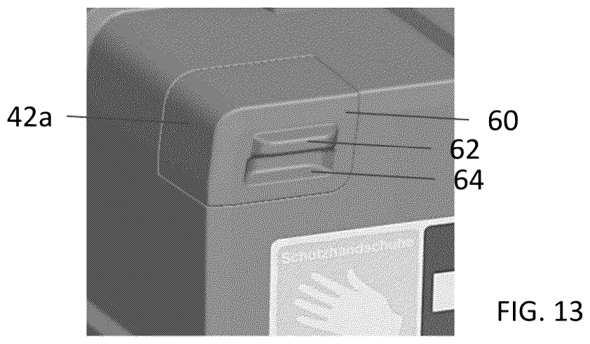

FIG. 13 shows a stacking centering apparatus on a left upper outer corner of the emergency case 200, i.e. on the cover cap 42a. The description of an exemplary configuration in this regard applies to all the stacking centering apparatuses. Proceeding from a smooth, flat surface that adjoins the surface outside the cover cap 42a, there is a projection 62, in this case having a longitudinal extension in parallel with the upper edge of the case shell 10. Below the projection 62, there is a recess 64 which is complementary in shape to the projection 62. The roles of projection and recess are swapped in the stacking centering apparatuses that are provided so as to be reversed. For example, on the right upper outer corner, there is a recess at the top and a projection below on the stacking centering apparatus 42b. Therefore, the projection of a case shell fits into the recess on another case shell of another emergency case.

In detail with regard to the stacking centering apparatuses: in the emergency case 100, the stacking centering apparatuses 30a and 30b, which are reversed relative to one another, are provided on the handle ends on the upper outer corners of the case shell 10. Stacking centering apparatuses 32a, 32b are in turn formed on the lower corners, wherein the stacking centering apparatus 32a is complementary to the stacking centering apparatus 32b. The stacking centering apparatus 32a is also complementary to the stacking centering apparatus 30a, and the stacking centering apparatus 32b is complementary to the stacking centering apparatus 30b (so as to be reversed).

So that open emergency cases can also be stacked, the inner lids 26 also comprise stacking centering apparatuses; see reference signs 34a, 34b and 36a, 36b on one inner lid. The stacking centering apparatuses 38a and 38b that are complementary thereto on the other case half and 40a, 40b are complementary in pairs to the stacking centering apparatuses: 38a to 34a, 38b to 34b, 40a to 36a, and 40b to 36b. In this way, the two inner lids 26 can also be effectively latched to one another, as shown in FIG. 6b.

In emergency cases with and without a handle, each case shell comprises partition elements for forming compartments. An open emergency case half is shown in FIG. 6d. The partition elements used therein are shown individually in FIGS. 6e to 6h, and the partition element from FIG. 6f is shown in an enlarged manner in FIG. 6i.

In the present case, as shown in FIG. 6d, a series of connection apparatuses 66 is provided on all four inner walls of the rectangular case shell. The connection apparatuses 66 have dovetail-shaped inlets, into which the partition elements 300a, 300b, 300c, 300d can each be inserted. As shown in FIGS. 6e to 6h, each of the partition elements 300a, 300b, 300c, 300d comprises a strip-shaped main body 310a, 310b, 310c, and 310d, respectively. In turn, there are connection apparatuses on each strip-shaped main body; in FIG. 6i, this is illustrated in particular for the partition element 300c, which comprises exactly two of these connection apparatuses 320-1 and 320-2. Each connection apparatus 320-1, 320-2 comprises a plurality of lugs, as can be seen in the present case on the basis of the connection apparatus 320-2: the lugs 322 alternate with one another on the right and left, with each lug 322 being opposite a cut-out 324, which is no more than a protrusion which again exactly corresponds to a lug on the rear face that can be imagined in FIG. 6i. Therefore, the cut-outs also alternate with one another on the right and left.

Each partition element comprises a sliding track 330. This can be inserted into the dovetail-shaped cut-outs in the connection apparatus 66 and thus can be directly fitted therein. By being able to be directly fitted to the inner walls of the case shell, this ensures that there is a particularly stable arrangement. So that the connection apparatus 66 does not weaken the case shell wall, there are reinforcement ribs 68, i.e. thickened portions or protrusions on the outer wall, which correspond exactly thereto on the outside.

The partition elements 300a, 300b, 300c, 300d have the lengths of a common multiple of a basic unit, namely the partition element 300a having five basic units, the partition element 300b having four basic units, the partition element 300c having three basic units, and the partition element 300d having two basic units. The compartmentalization according to FIG. 6d can be achieved by the partition element 300a, the partition element 300b, the partition element 300c, and the partition element 300d, each being simple to use. Here, the partition element 300a is inserted into opposite connection apparatuses on the inner walls, the partition element 300b is inserted into a connection apparatus on the partition element 300a on one side and a connection apparatus on the other inner wall of the emergency case half on the other side. The partition element 300c is inserted into the central connection apparatus of the partition element 300b by a sliding track and into a connection apparatus on the front inner wall of the emergency case half by the other sliding track, and lastly the partition element 300d is inserted into the central connection apparatus on the other side of the partition element 300b from the partition element 300c by a sliding track, and the partition element 300d is inserted into a connection apparatus 66 on the rear inner wall of the emergency case half by the other sliding track.

FIG. 6k shows that, by selecting the suitable size ratios in a series of emergency cases 100a, 100b, and 100c of different sizes, said partition elements can be reused over and over again. In a measurement direction with the same height, the upper emergency case 100a has an extension of five units, the middle emergency case 100b has an extension of four units, and the lower, smallest emergency case has an extension of three units. Therefore, in the emergency cases, the same partition elements can be used over and over again, which simplifies the production of different series.

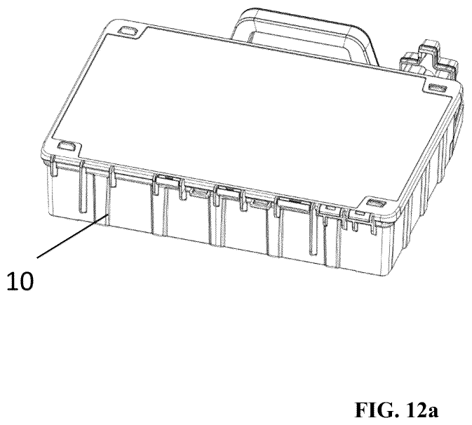

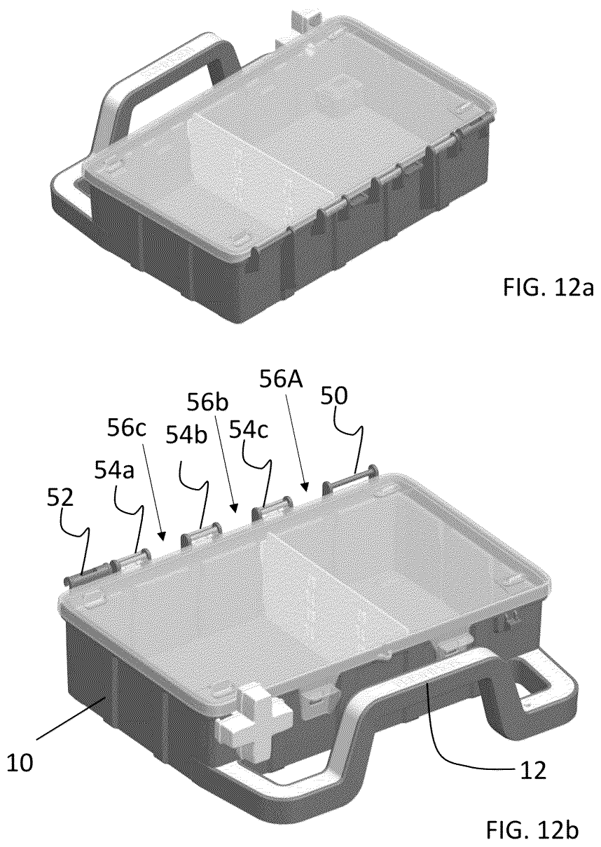

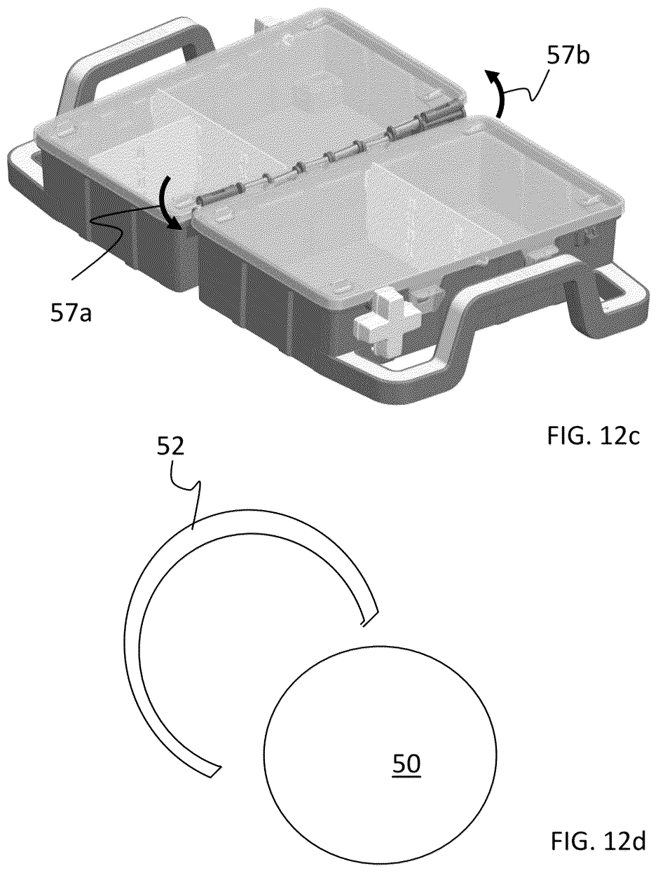

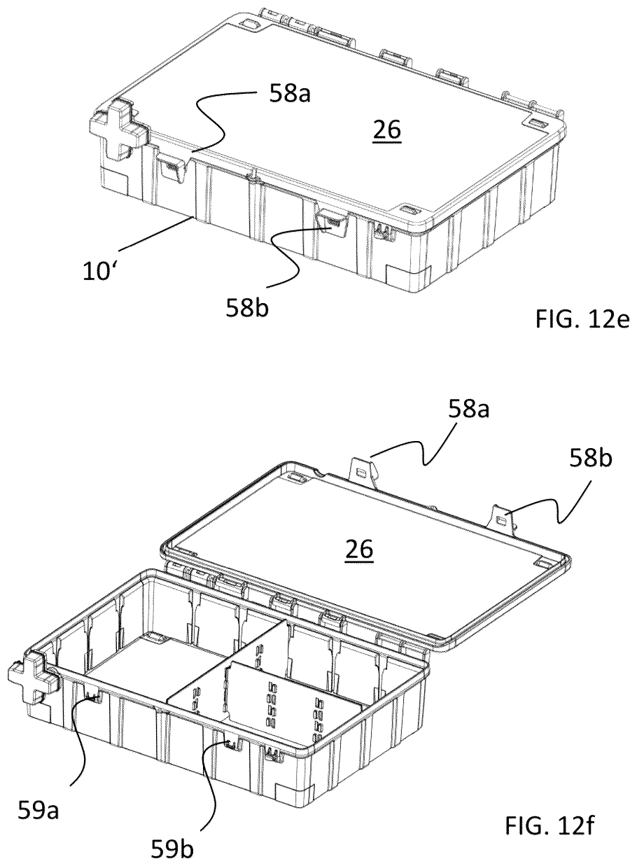

The two emergency case halves comprising the case shells 10 and the handle 12 in the emergency case 100 from FIGS. 1 to 6c and the corresponding case halves of the emergency case 200 can also be used on its own, as shown in FIGS. 12a and 12b. In particular, the two emergency case halves in the emergency cases 100 and 200 are detachable from one another. Connection devices that are complementary to one another are provided in the lower region of the respective emergency case half: a rod 50 on the lower region of the emergency case half comprising the shell 10 and the handle 12 can be inserted into the resilient connection device 52, which is C-shaped in cross section, of an identically constructed emergency case half. The apparatuses 54a, 54b, and 54c for fastening the inner lid are also located along the axis. The corresponding apparatuses can be fitted into the gaps 56a, 56b, 56c in the complementary emergency case half, such that a closed axis of rotation is formed.

FIG. 12c illustrates the situation in which the two emergency case halves 10 are just being interconnected: here, the rod 50 of one emergency case half is inserted into the connection device 52 of the identically constructed other emergency case half (see arrow 57a) and, accordingly, the rod 50 of the other emergency case half is inserted into the connection device 52 of the one emergency case half (see arrow 57b).

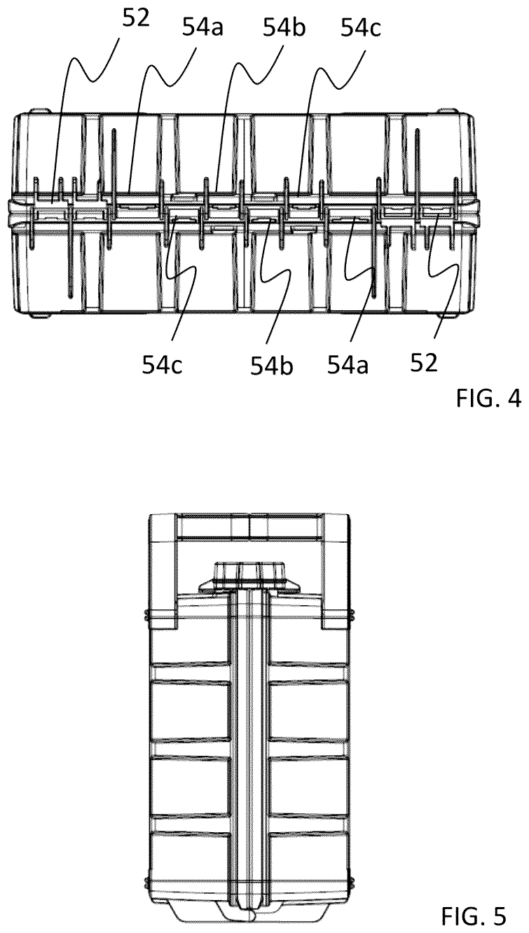

FIG. 4 shows how the thus assembled emergency case 100 looks from the outside. (The same applies to the emergency case 200:) The gaps 56a, 56b, 56c can no longer be seen, but instead the hinges 54a, 54b, 54c, to which the corresponding C-shaped resilient connection elements of the inner lid 26 are attached, are each joined to the complementary emergency case half.

FIG. 12d is a cross section of the rod 50 beside the connection device 52. By pulling the connection device 52 open, the rod can be inserted therein, and is retained therein once said device has sprung back.

FIGS. 12e and 12f show a case shell 10′ without a handle. Except for the teaching relating to the handle, that which is set out here in relation to the emergency case half or case shell 10 is applicable to the above case shell. That which is set out here in relation to the case shell 10′ is applicable to the case shell 10, unless it relates to the omission of the handle.

The clamping tabs 58a and 58b are formed in one piece with the remaining portion of the inner lid 26. Alternatively, they are added onto an inner lid. The clamping tab 58a corresponds to the latching lug 59a to which it can be latched, and the clamping tab 58b corresponds to the latching lug 59b to which it can be latched. Therefore, the inner lid 26 can be securely closed so that the respective case shell 10 or also 10′ can be carried on its own without anything falling out.

The inner lid 26 is fastened to the case shell 10 using the same clamping principle as described above with reference to FIG. 12d: in the region of the hinges 54a, 54b, and 54c, a rod to which a C-shaped resilient connection device of the inner lid is securely clamped is formed on the case shell 10 or 10′ in each case. These C-shaped resilient connection devices are all formed in one piece with the remaining portion of the inner lid 26. Alternatively, they are added onto an inner lid.

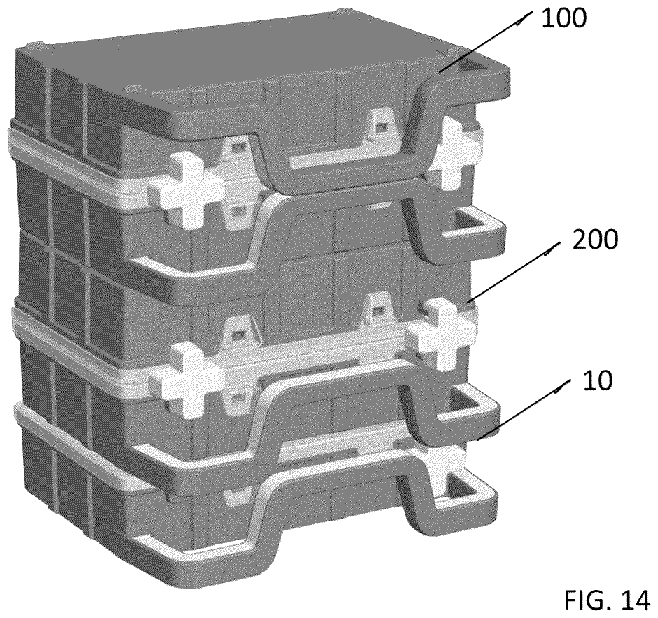

FIG. 14 shows a stack of a) the emergency case as in FIG. 12a, b) the emergency case 200 from FIG. 7a, and the emergency case 100 from FIG. 1. The stacking centering apparatuses contribute to the stability.

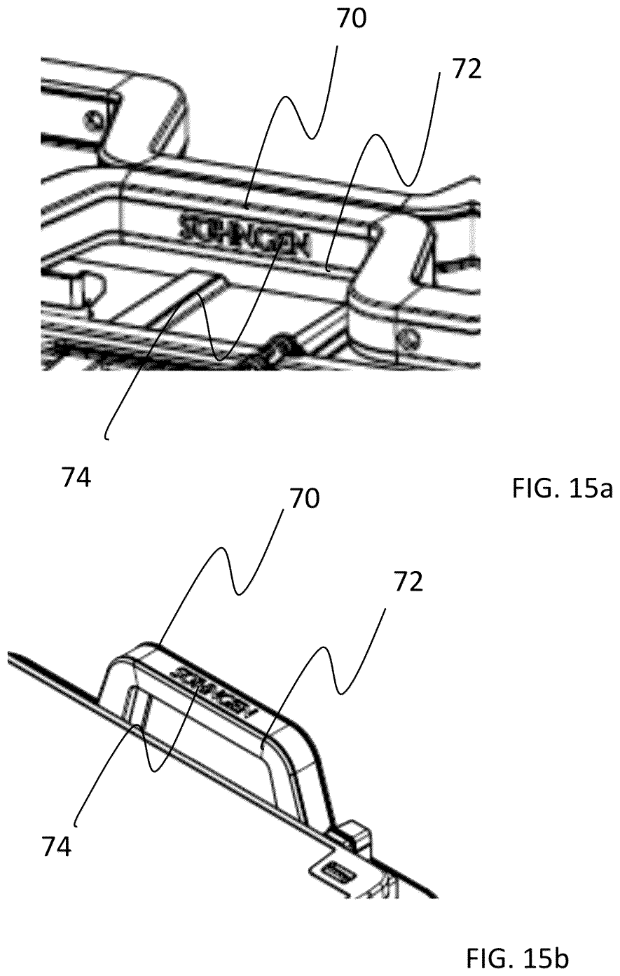

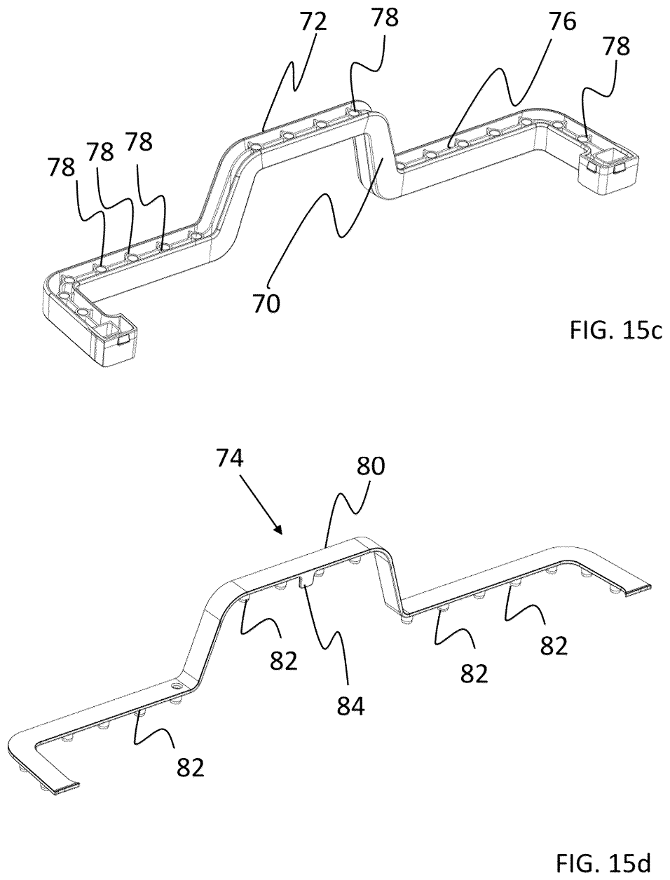

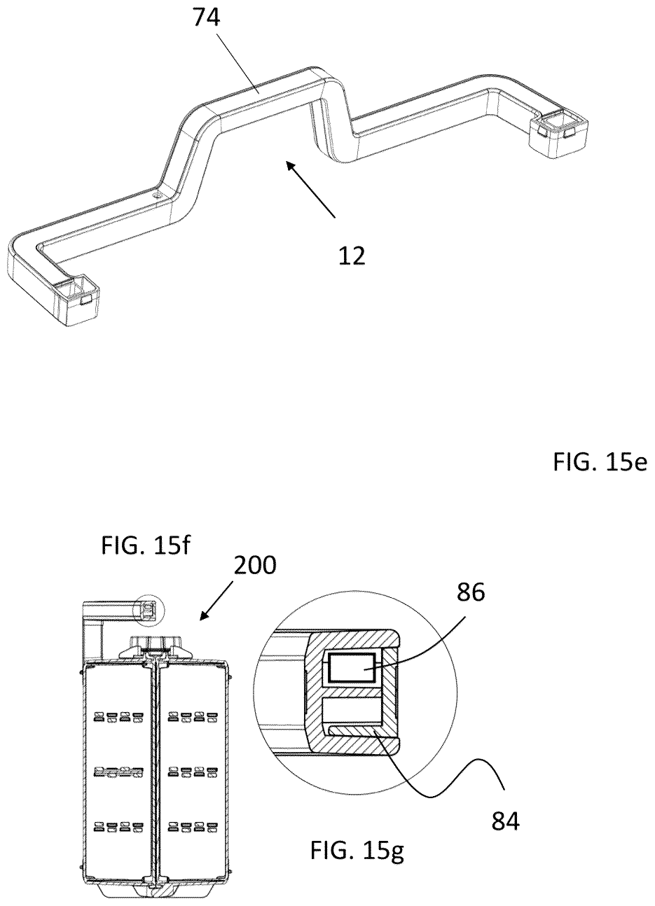

FIG. 15a is a detail of the handle from FIG. 1, as viewed from the outside. This figure shows that the handle comprises a frame having an upper frame part 70 and a lower frame part 72. An insert part 74 of the handle is inserted between the two frame parts 70 and 72. The insert part 74 can be glued or pressed in, and optionally can also be clipped to elements located in the inside of the handle.

FIG. 15b is a detail of the handle from FIG. 6a, which is visible therein from its inner face. Here too, the insert part 74 can be seen between the frame parts 70 and 72. It can be the same insert part as in FIG. 15a. Alternatively, two insert parts can be provided (not shown).

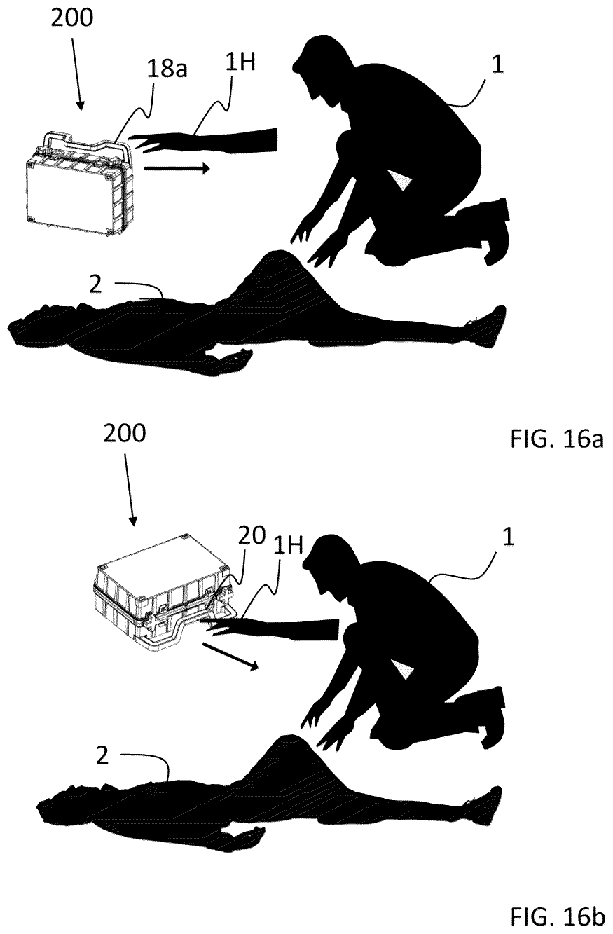

FIG. 16a illustrates how a first aider 1 kneels beside a patient 2. The emergency case 200 comprising a handle 12 can be grasped by the first aider 1 using their hand 1H on the portion 18c, and when they stand up, it is pulled closer to them.

According to FIG. 16b, in the other situation, the first aider 1 uses their hand 1H to pull over the emergency case 200 that is lying down by grasping it on the portion 20.

FIG. 15c shows the handle from FIG. 15b on its own, without the insert part, i.e. only the main body of the handle, which is preferably injection-molded from plastics material. A cavity 76 is visible in the main body. It is open to one side, namely toward the upper face in the present case. In the main body, a series of circular cut-outs 78 is provided over the entire above-described extension (first region, second region, each in duplicate, and a third region therebetween).

The insert part 74, can also be referred to as a closure cap, is shown on its own in FIG. 15d as a whole. Studs 82 which can be inserted into the cut-outs correspond to the circular cut-outs 78 on the main body, the latching taking place in a simple manner. They are distributed over the strip-shaped main body 80 of the insert part/the closure cap 74. There is an insert tab 84 in the center, corresponding to the third region of the handle.

As shown in the sectional view according to FIG. 15f, the insert tab 84 ensures that an electronics module 86 introduced into the cavity 76 in the main body is particularly effectively retained. This can for example comprise a memory, in which it is indicated what materials should or need to be contained in the emergency case/first aid case. The electronics module 86 can allow this information to be read out wirelessly and, for example, by means of a query signal, can obtain energy for sending a response signal by means of which this information is output.

The emergency case half can be carried on its own, also because the inner lid 26 is suitable for being latched to the case shell 10 or 10′. This can, for example, be implemented by a peripheral lip on the inner lid, which simply continuously engages over the frame of the case shell 10, 10′. Because the inner lid 26 is opaque, the material in the dressing case is protected against aging due to ultraviolet radiation. Third parties also cannot see into the interior of the emergency case half when it is being carried on its own. Alternatively, the inner lid is configured to be transparent.

The insert part 74 can be configured to be fluorescent, for example by adding fluorescent particles, as marketed by American Permalight Corp. under the brand name PERMALIGHT®. The insert part also bears a logo in the example. This means that the handle is also clearly visible at night, for example to the doctor kneeling beside the patient who wants to pull it toward them.

The emergency case can also be configured to be a different color from the insert part.

It can also be fluorescent, preferably less fluorescent than the insert part of the handle. In the simplest case, a fluorescent film can be applied, and then a textured pattern can be made to glow.

Furthermore, the inner lid 26 can likewise be fluorescent so that an emergency case half can also be identified particularly easily on its own.

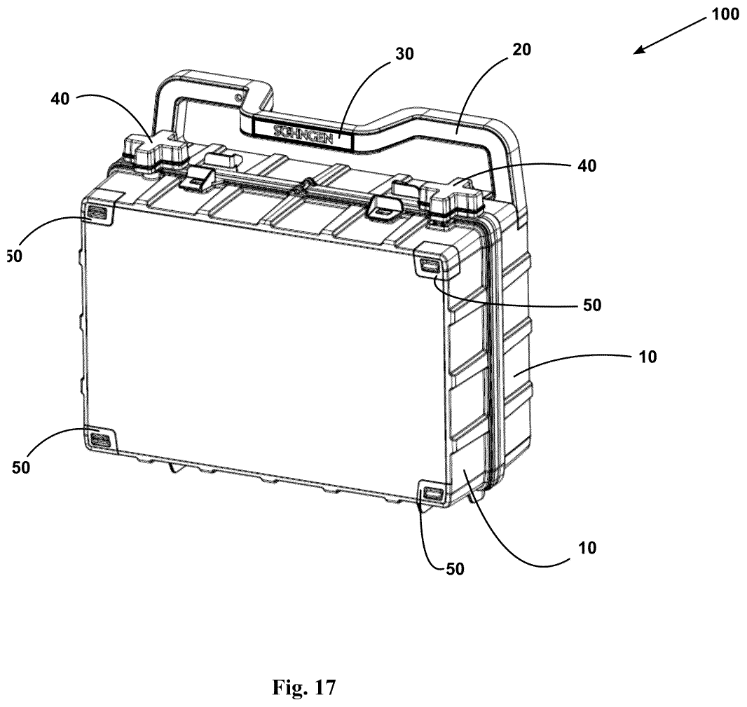

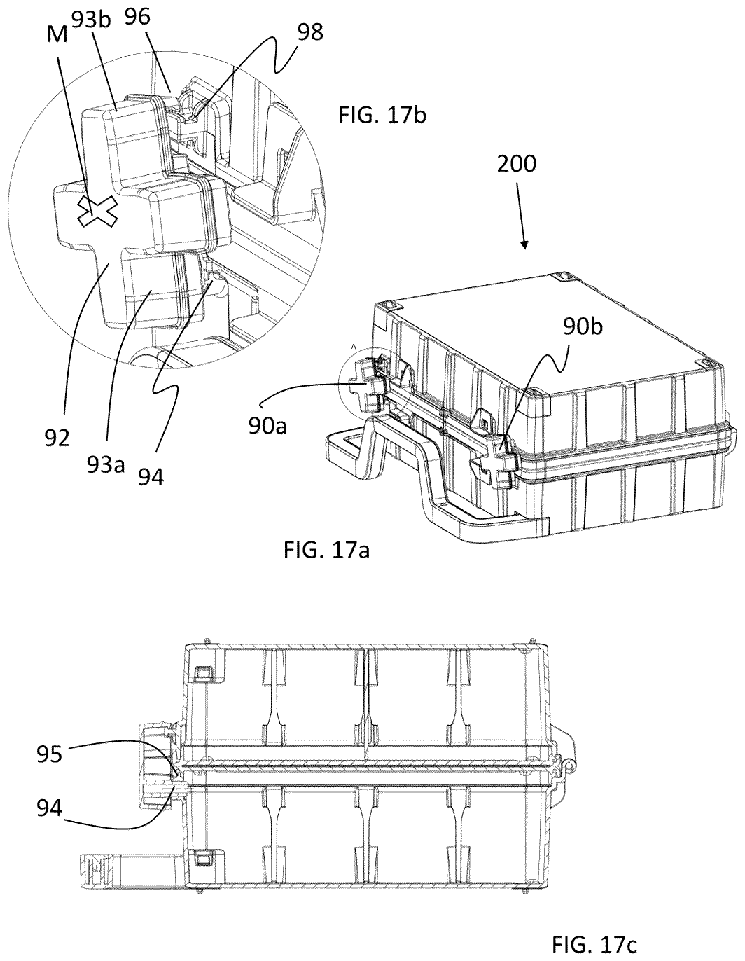

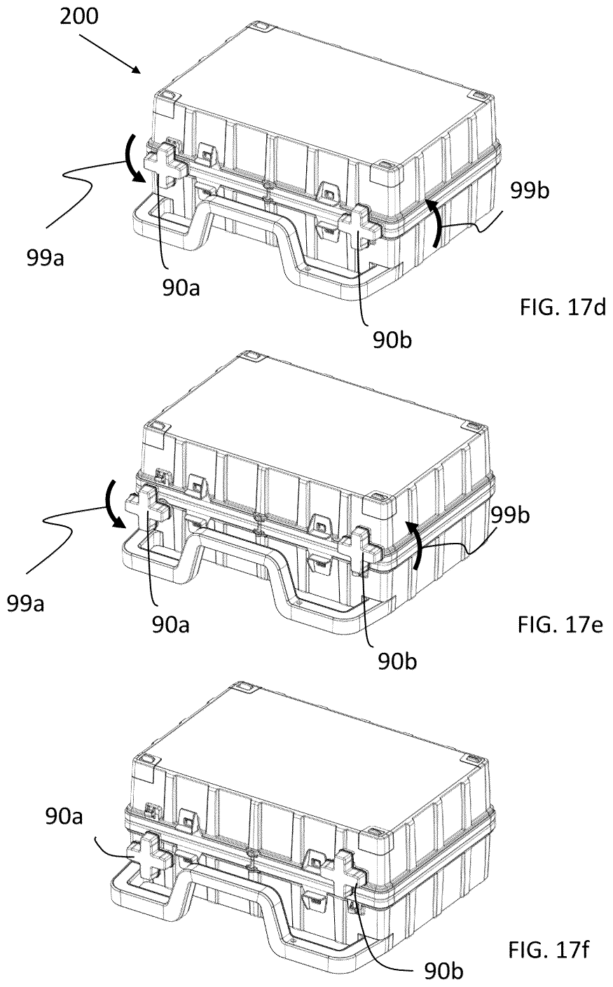

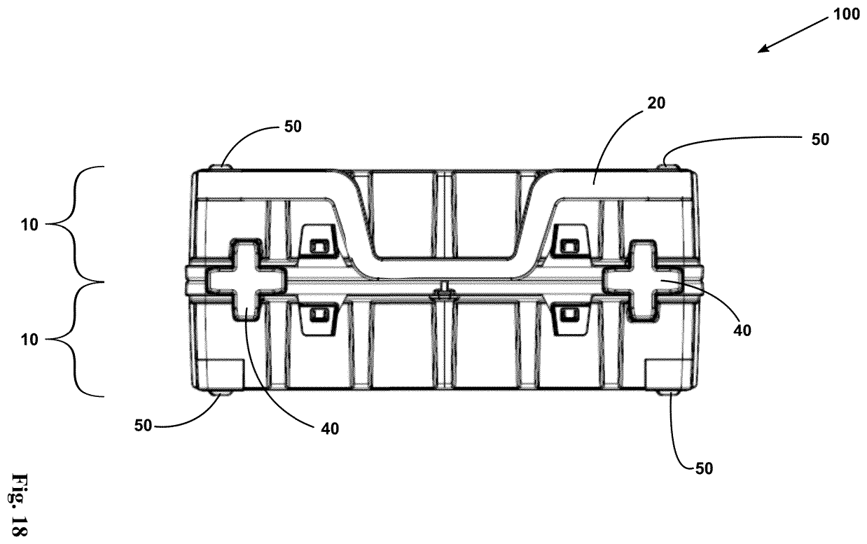

FIG. 17 shows a first aid kit according to the invention, designated 100. The case shells 10 do not contain any phosphorescent material. On the other hand, the handle 20, the insert 30, the closures 40 and the stack centering devices 50 contain phosphorescent material. The design of the handle 20 is expressly not limited to the shape shown in FIG. 1. FIG. 1 clearly shows that the phosphorescent design of individual sections, in particular the phosphorescent design of the attachable parts handle 20, insert 30, closures 40 and stack centering devices 50, already increases the visibility of the case, even if it is mounted, for example, in a wall mount at eye level, which is located on the side of the first aid case facing away from the viewer. The handle 20 is shown here, in abbreviated form, in an S-shape. Other shapes of the handle 20 are also conceivable, such as simple handle grips.

The emergency case can be opened and closed in a particularly simple manner owing to the design of the knob apparatuses 90a, 90b. For this purpose, FIG. 17a shows the emergency case 200 in the state in which the knob apparatuses are just being opened. FIG. 17b is the enlarged circular detail from FIG. 17a.

It shows that each knob apparatus 90a and 90b substantially comprises three components, namely the actual knob 92, which is cross-shaped in the present case. The cross shape has legs of equal length that are at an angle of 90° to one another, of which the lower leg 93a and the upper leg 93b are opposite one another. The knob 92 is flat on the front face of the cross shape and has side walls that are substantially perpendicular to the front flat surface on each leg.

The second component is the hollow shaft 94, which is formed on the leg 93a and is inserted into a flange 95 on the associated case shell.

The third component of the knob apparatus 90a, 90b is a rear-engaging element in the form of a rear-engaging lug 96, which can also be called a rear-engaging hook or a rear-engaging claw. During closing, the rear-engaging lug 96 engages behind the latching lug 98 on the other case shell that is not associated with the knob apparatus. When setting down the case, the front left knob apparatus is associated with the lower case shell, and the front right knob apparatus is associated with the upper case shell. Accordingly, the latching lug 98 behind which the left knob apparatus 90a engages is formed on the upper case shell and a corresponding latching lug 98 behind which the right knob apparatus 90b engages is formed on the lower case shell (the latter not being shown in this figure).

With regard to these latching lugs 98, it should also be noted that they are preferably formed as in FIG. 17b: On the case shell (in FIG. 17b, the upper case shell), the side wall is reinforced (“base plate” for the latching lug), on which there is then also a pair of feet, from which (preferably substantially perpendicularly to the surface of the front wall of the case shell and therefore the “base plate”) a small arm projects which bears the latching lug. The rear-engaging lug 96 is pivotable by means of the arm and, owing to the rounded shape of the feet, the rear-engaging lug 96 can pull the other case shell (the upper case shell in FIG. 17b) slightly downward, such that the emergency case is closed in a sealed manner.

It is noteworthy that, owing to its legs of equal length, the cross shape has its geometric midpoint M exactly in the center, the point of intersection of the two lines forming the cross. According to the arrangement of the hollow shaft 94 on one of the legs, the knob apparatus is therefore eccentrically mounted.

This eccentric mounting has the advantage that the knobs are pivotable to a considerably greater extent than they would be with different mounting. In particular, they are even pivotable, and not just rotatable.

FIG. 17d shows the emergency case 200 in the closed state. The arrows 99a and 99b show the pivot direction in which the knobs 92 of the knob apparatus 90a or 90b have to be pivoted. In a transition to FIG. 17e, the rear-engaging lug 96 pivots behind the latching lug 98 into the open position shown in FIG. 17e. In the latter figure, the emergency case can already be open.

According to further rotation in the direction according to the arrows 99a and 99b, a stowed position, as shown in FIG. 17f, can also be reached. This stowed position has the advantage that the emergency case can be opened more easily and, in particular, the knob apparatuses cause less disruption. This is particularly helpful when the emergency case 200, as set out above, is intended to be divided into two emergency case halves.

In the transition from FIG. 17d to FIG. 17e, i.e. from the closed position into the rest position, the knob is rotated (pivoted) by 90°. In the further transition to the stowed position according to FIG. 17f, a further rotation by another 90° is performed. Overall, from the closed position into the stowed position, the knob is pivoted by 180°.

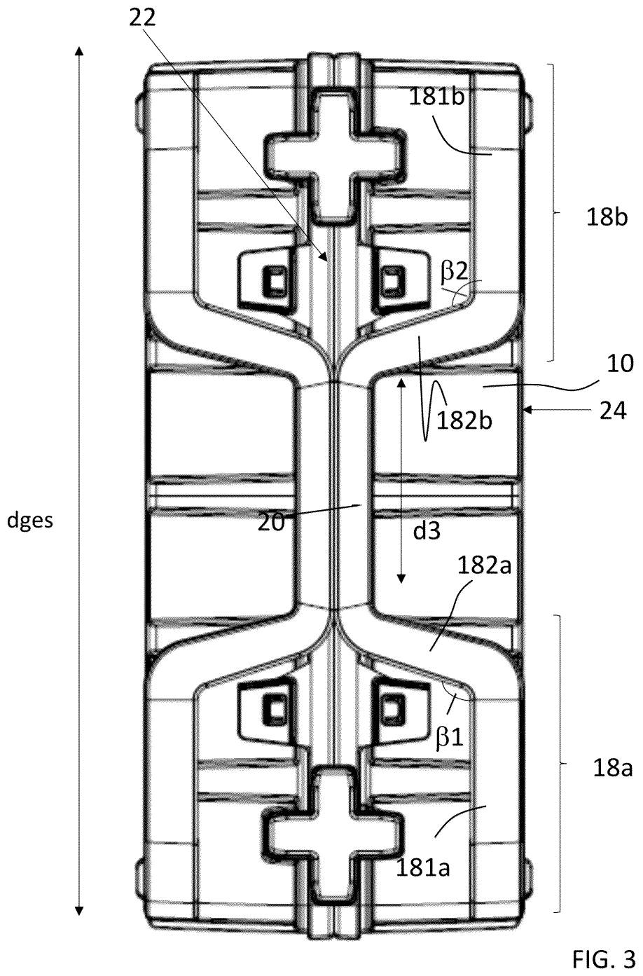

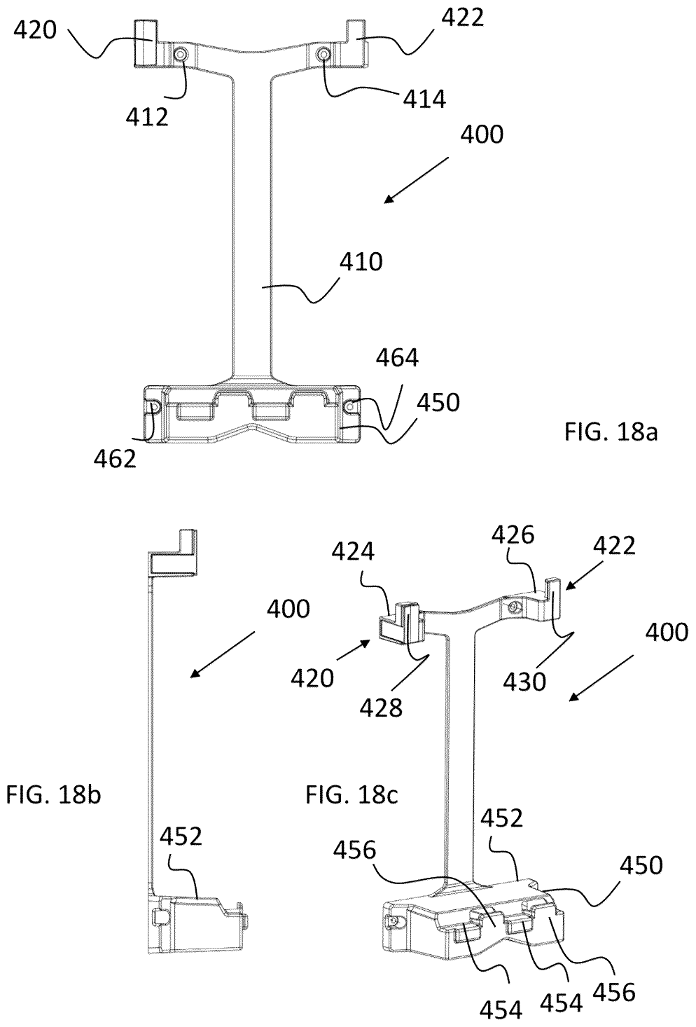

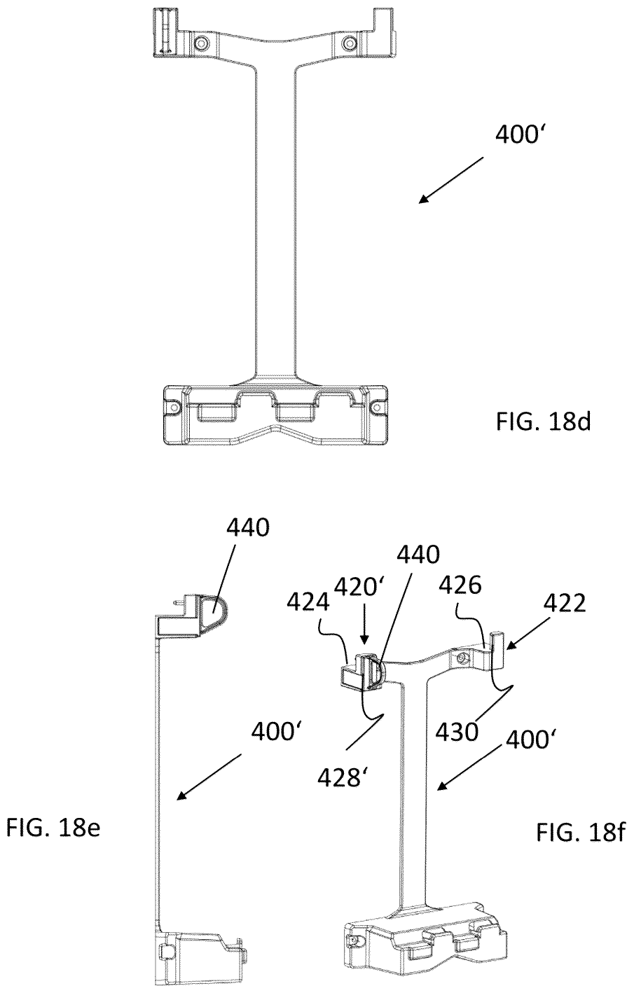

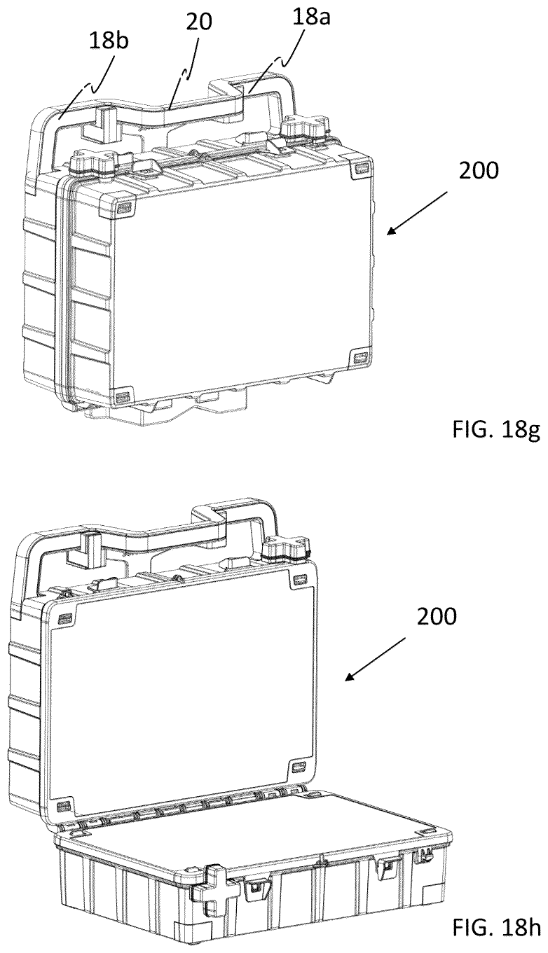

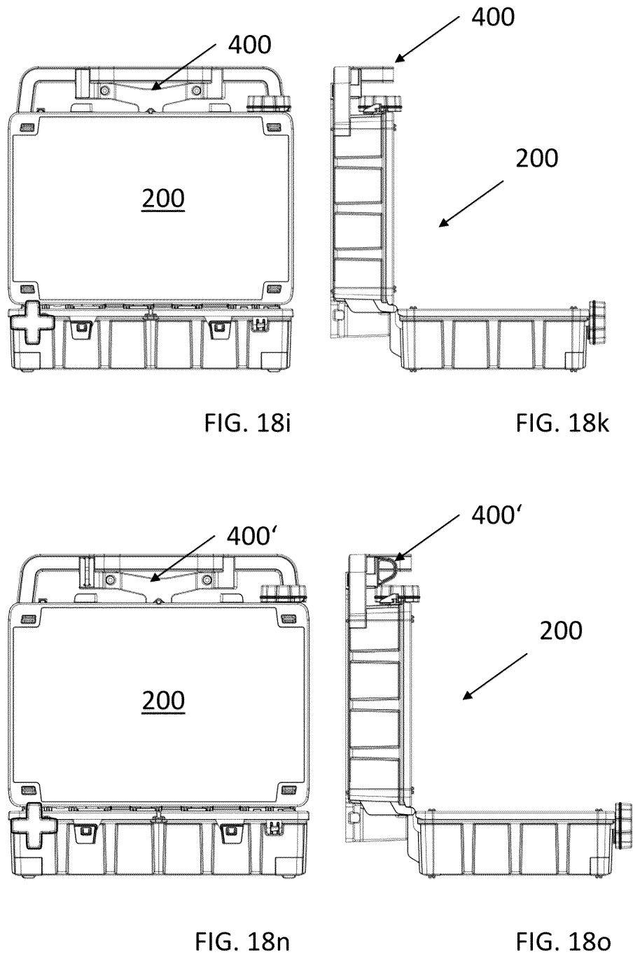

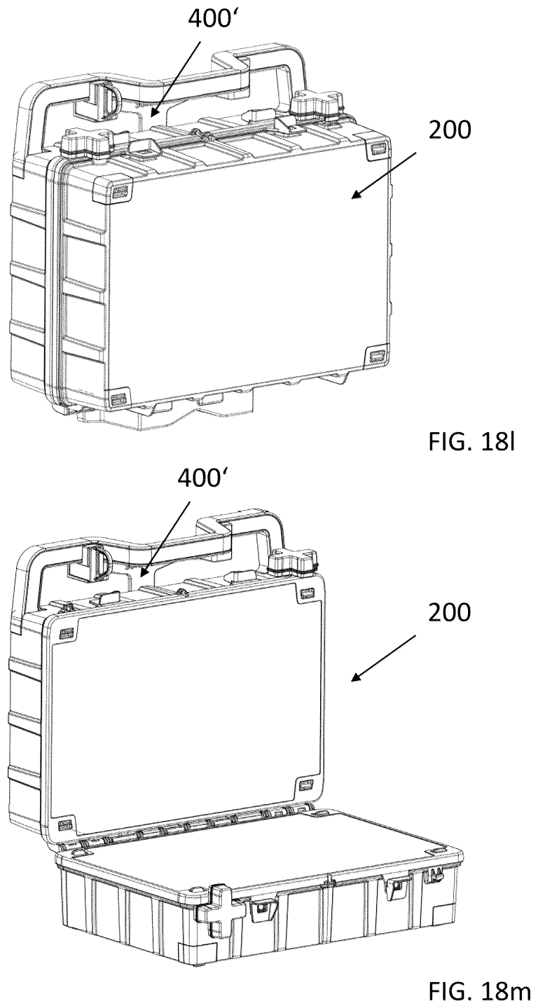

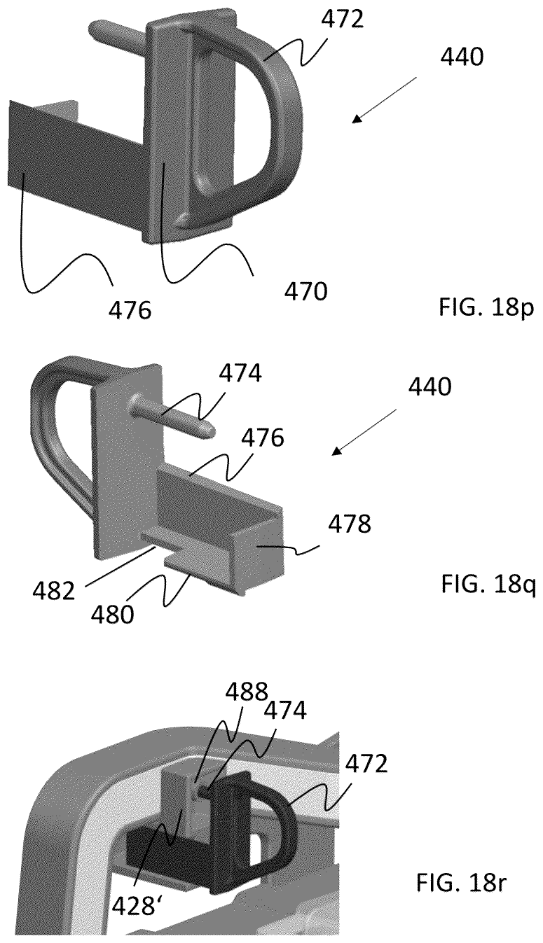

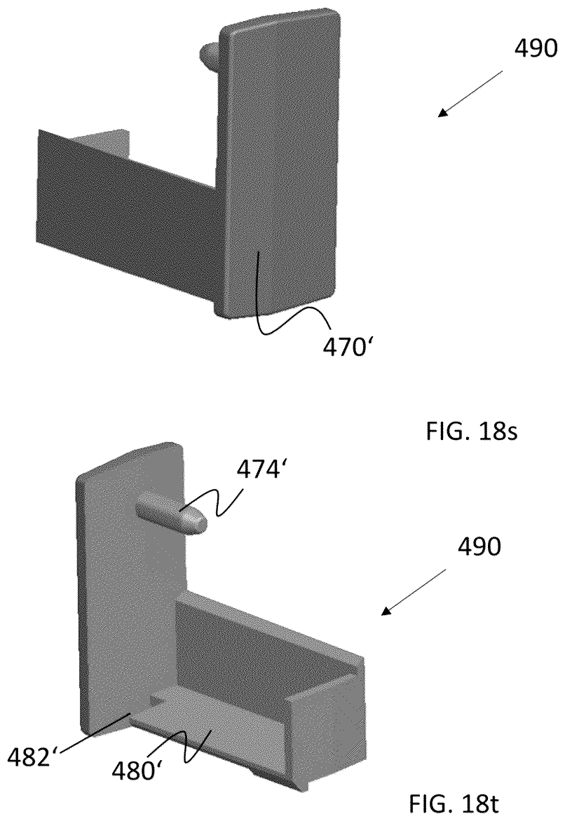

FIG. 18 illustrates from the front view that the handle 20, the closures 40 and also the stack centering devices 50 increase the visibility of the case due to their phosphorescent design, even if it is at eye level on a shelf, for example. The case shells 10 do not contain any phosphorescent material. The insert 30 is not shown in FIG. 2.

FIGS. 18a, 18b, and 18c show a wall bracket 400 that can be used in connection with the above-described emergency cases 100 and 200 or emergency case half thereof, and FIGS. 18d, 18e, and 18f show the same wall bracket, but with a lock.

Except for the configuration of the left finger once with and once without a lock, the configurations of the wall bracket 400 also entirely apply to the wall bracket 400′ with a lock.

The wall bracket 400 comprises a rear bar, which is T-shaped, i.e. comprises a straight bar of substantially the same width, from which two smaller bars branch off, in the present case to be slightly bent upward. At the end of the two smaller bars of the T shape, a thickened shoulder for drill holes 412, 414 is provided, through which threaded bolts can be guided in order to fasten the wall bracket 400 to a wall (not shown). The thickened portions for the drill holes 412, 414 already belong to the retaining arms 420 (left) and 422 (right), which are the same length in the present case, i.e. are configured to be symmetrical to one another. As particularly clearly shown in FIG. 18c, the retaining arm 420 provides a seat 424 for a portion of the emergency case handle, and the retaining arm 422 provides a corresponding seat 426 for another portion of the handle. The retaining arm 420 further comprises the retaining finger 428, and the retaining arm 422 comprises the retaining finger 430.

As shown in FIG. 18g, in particular the region 18a of the handle 12 of the emergency case 200 can be placed onto the seat 426 of the right retaining arm 422 and the second region 18b of the handle can be placed onto the seat 424 of the left retaining arm 420 of the wall bracket 400. Furthermore, FIG. 18g also shows that, owing to the sufficient distance between the fingers 428, 430 of the retaining arms 420 and 422, the third region 20 between the two second regions 18a and 18b of the handle is particularly easily accessible, and this is made easier by the shape of the handle (the third portion extends along the inner longitudinal edge). The entire case can be taken down in one go by grasping the third portion 20. In the case not shown in the figure in which the emergency case 100 comprising two handles is used, the emergency case can of course be grasped by the two third regions 20 together, similar to that described above in FIG. 16a for the first aider.

As also shown in FIGS. 18a, 18b, and 18c, the wall bracket 400 comprises a stop 450 for the emergency case. Drill holes 462, 464 are also provided here. The stop 450 first of all contains a seat 452, on which the rear (i.e. wall-side) emergency case half can sit. In the front region, the stop 450 comprises protrusions 454 and projections 456, of which the dimensions correspond exactly to the ribs on the case shell of the emergency case; see FIG. 6d and the associated description of the ribs 68 matching to the connection apparatuses 66.

In this way, the emergency case can be retained in a particularly effective and stable manner in the wall bracket in the opened-up state, as shown in FIGS. 18h, 18i, and 18k, and can be brought from the non-opened-up state according to FIG. 18g into the opened-up state according to FIG. 18h.

The wall bracket 400′ shown in FIG. 18d, 18e, 18f as well as 181, 18m, 18n, 180 differs from the wall bracket 400 only in that the fastener (lock) 440 is provided on the finer 428′ of the retaining arm 420; see in particular FIG. 18f and FIGS. 18p, 18q, and 18r.

In this embodiment, the emergency case, as shown in FIG. 18l, is secured to the wall bracket by the wall-side, rear emergency case half by means of the fastener/lock 440. Therefore, nobody can remove this emergency case half from the wall without opening the lock. This ensures that corresponding first aid material and the like is available, which is contained in the emergency case half. In the opened-up state according to FIG. 18m, however, the front emergency case half that has been opened downward can be separated from the rear emergency case half in the manner described above with reference to FIGS. 12a to 12d and can later be reattached thereto. Therefore, in the room in which the wall bracket is fastened to the wall, it is ensured that one emergency case half remains in the room in any event, while material urgently required by the first aider can be provided by the other emergency case half. Unlike in FIGS. 18l and 18m, the emergency case 100 can also be used such that the separated emergency case half can be provided with a handle.

The following is to be stated with regard to the fastener 440: the fastener 440 is provided in the form of a fastener cap, which is fitted to the finger 428′ and is shown separately as a whole in FIGS. 18p and 18q.

The fastener cap 440 comprises a front plate 470, on which a pull tab 472 (in the shape of a loop here) is formed. On the rear of the front plate 470 is a pin 474, which is supported to engage in a cut-out (“hole”) at a suitable point on the emergency case. On a preferably outer side in the wall bracket (and only on this side), there is a side plate 476, at the rear there is a rear plate 478 for support on the emergency case, and there is a base plate 480, which reinforces the assembly. A cut-out 482 in the base plate 480 leaves space for the foot of the finger 428′.

As shown in FIG. 18r, the entire fastener cap 440 can be pulled forward in one, in order to thus be able to bring the pin 474 out of engagement with the emergency case and remove said case from the wall bracket.

It is advantageous for a spring (not shown in the drawings) on which the fastener cap 440 is supported and which is supported on the retaining arm 420′ at the other end to pull the fastener cap 440, after being pulled forward, backward (toward the wall) again in a restoring manner, i.e. into the finger 428′ of the wall bracket.

FIG. 18r also illustrates that the pin 474 first engages in a cut-out (through-hole) 488 on the finger 428′ and can engage in a cut-out (not shown) in the emergency case therebehind.

If a fastener is not required, the fastener cap 440 can simply be omitted.

For practical and/or aesthetic reasons, a cover cap 490 can be provided, which is shown separately as a whole in FIGS. 18s and 18t and can be accordingly inserted on the finger 428.

The cover cap 490 differs from the fastener cap 440 substantially only in that a pull tab is not provided on the front plate 470′, and therefore this front plate 470′ has a flat surface, and in that the pin 474′ is shorter than the pin 474, since the pin 474′ is not supposed to engage in a cut-out in the emergency case, but is merely supposed to ensure that the cover cap 490 is retained on the finger 428, i.e. can only be inserted into a cut-out (such as into the cut-out 488 from FIG. 18r). Optionally, the base plate 480′ is also provided with a smaller cut-out 482′ than the cut-out 482 in the fastener cap 440. No clearance needs to be provided for greater movability. The base plate 480′ then has only a small amount of clearance relative to the finger 428; it can be fastened in an interlocking fit where necessary.

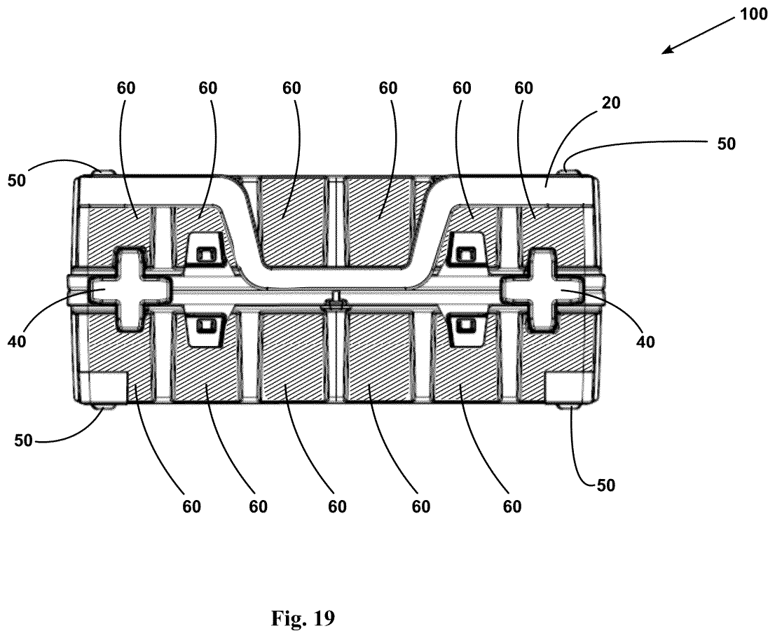

FIG. 19 shows a first aid kit 100 whose handle 20, fasteners 40 and stack centering devices 50 contain phosphorescent material. The insert part 30 cannot be seen in FIG. 3. The metallized surface sections 60 reflect the light emitted by the aforementioned add-on parts to an even greater extent than would be the case with conventional plastic surfaces, thus improving the visibility of the first aid kit 100 in general and the visibility of the add-on parts in particular. It is also conceivable that the surfaces of-preferably raised-elements such as “first aid” or “first aid kit” lettering or symbols such as a first aid cross could be metallized. As a result, these elements stand out visually from the surrounding phosphorescent sections and thus become visible to the first aider, who can more quickly deduce the contents of the case. This is particularly conceivable with phosphorescent case shells, as particularly large lettering or symbol elements with a metallized surface can be implemented here. However, a first-aid cross with a metallized surface could also be arranged in the handle area of the first-aid kit, for example, so that it stands out visually from a phosphorescent wall and is thus easily visible to the first aider treating the patient.

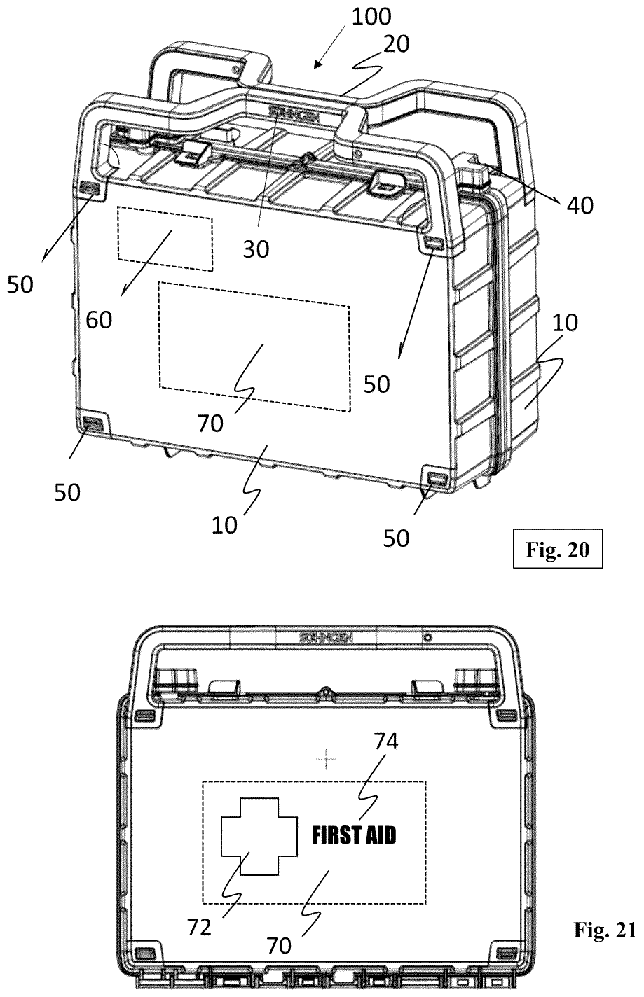

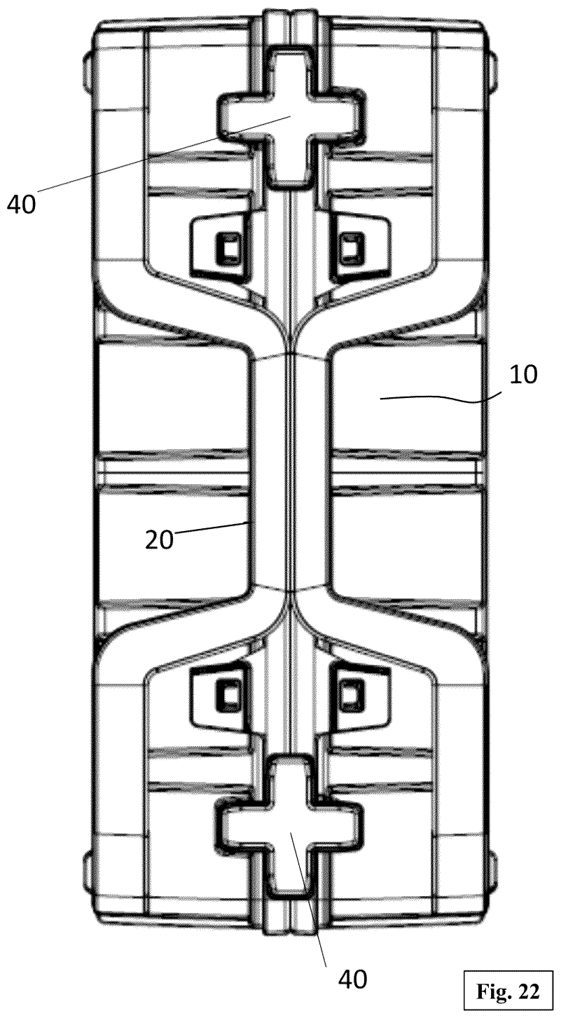

FIG. 20 shows a first aid case according to the invention, denoted by 100 as a whole. The case shells 10 do not contain any phosphorescent material. However, the handle 20, the insert part 30, the closures 40, and the stacking centering apparatuses 50 can contain phosphorescent material. The configuration of the handle 20 is expressly not limited to the shape shown in FIG. 20. It is clear from FIG. 20 that the phosphorescent configuration of individual sections, in particular the phosphorescent configuration of the add-on parts that can be affixed, namely the handle 20, the insert part 30, the closures 40, and the stacking centering apparatuses 50, increases the visibility of the case, even if it is fastened at eye level in a wall bracket which is located on the side of the first aid case facing away from the observer. In this case, the handle 20 is, in short, shown in an S shape. Other shapes of the handle 20 are also conceivable, such as simple loop handles. In the present case, information in the form of a symbol and lettering is provided at least in one area 70 by means of a hot stamping process. This is shown in detail in FIG. 21: a cross 72 is provided here as the symbol and the lettering “First Aid”, denoted by 74, is provided as a second piece of information. Attention is also drawn to the area 60, shown by way of example, in which surface sections are metal-coated. Any surface sections on the case shells and/or add-on parts can be metal-coated.

Beyond the area 70 shown in FIG. 21, add-on parts can also be provided with a phosphorescent material in a hot stamping process. Here, for example, the handle 20 and/or the cross-shaped closures 40 also act as the stacking centering apparatuses.

By way of the plan view, FIG. 22 shows that, in the optional phosphorescent configuration of the handle 20, the closures 40, and also the stacking centering apparatuses 50, the visibility of the case is increased, even if it is on a shelf at eye level, for example. The case shells 10 do not have to contain any phosphorescent material.

Overall, by means of the phosphorescent material that has been applied by means of a hot stamping process, the first aid case is more clearly visible to the first aider, meaning that they can deduce the contents of the case more rapidly.

The hot stamping known per se can, for example, be described using a device such as that described in DE 10 2012 109 315 A1. This document describes how a non-flat surface can be stamped. It goes without saying that, for a stamped flat surface, the process is actually somewhat easier.

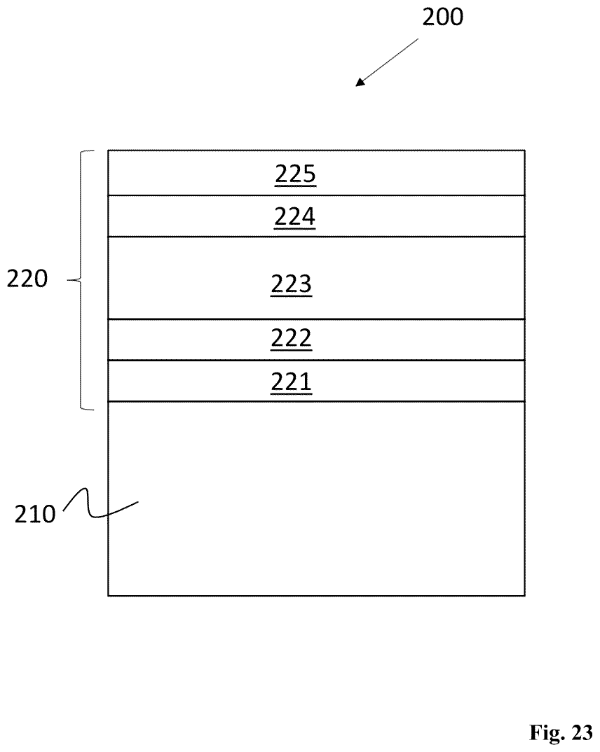

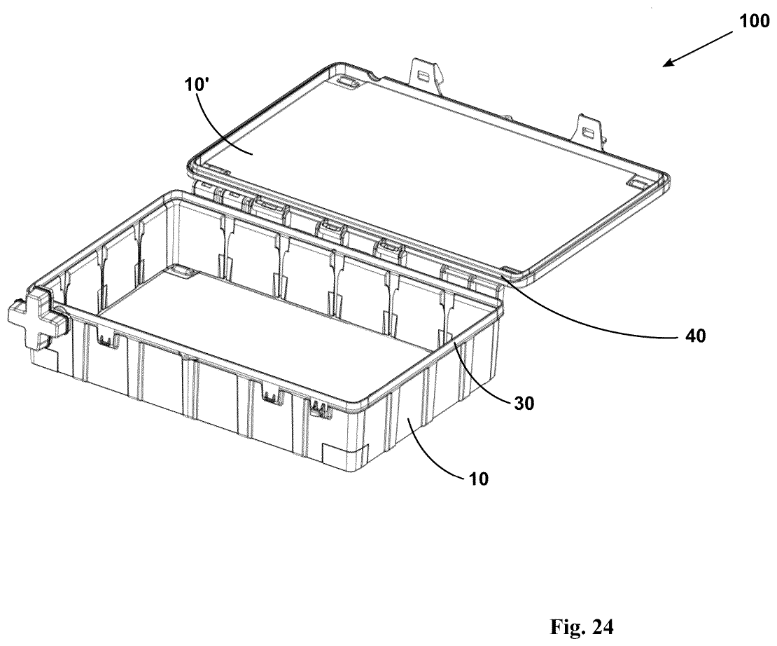

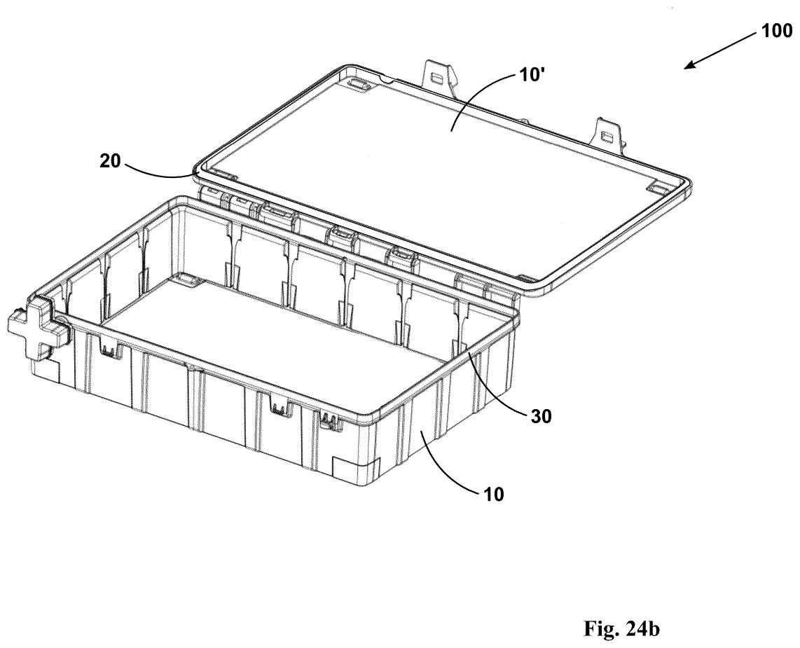

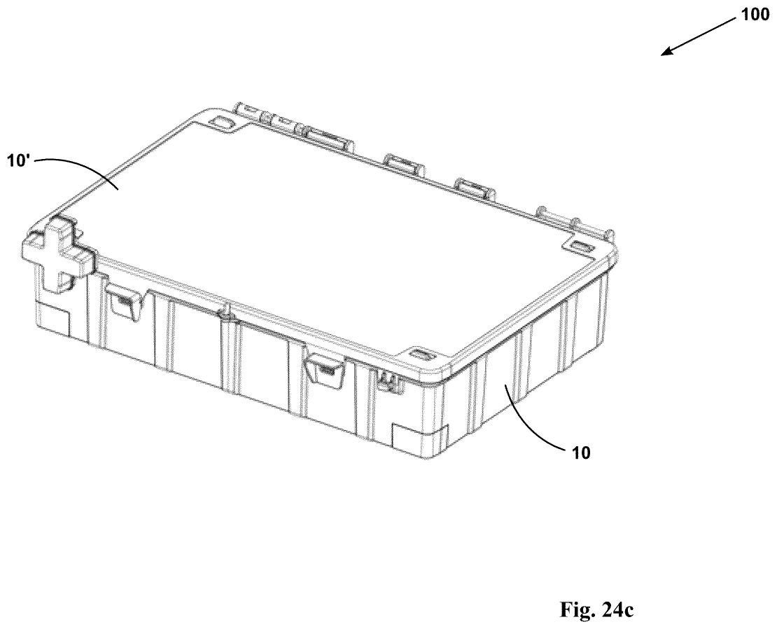

The hot stamping foil used in the invention is shown in FIG. 23, and is denoted by 200 as a whole therein. It comprises a carrier foil 210 and a transfer sheet 220. As far as layers and/or sheets are being discussed in the present case, this can also always be a multi-layered structure which amounts to a layer/sheet overall.

The carrier foil 210 consists of polyester material, e.g. PET, of polypropylene, polystyrene, PVC, PMMA, ABS, and/or polyamide.

The transfer sheet 220 comprises a plurality of sub-sheets or sub-layers, namely first of all the separation layer 221, which consists of wax or wax-containing compounds which melt during the hot stamping and thus ensure that the transfer sheet 220 becomes detached from the carrier foil 210 as a whole at the stamped points, i.e. in a structured manner. There is also a protective coating layer 223, which is provided with phosphorescent particles having a particle diameter d90≤5 μm in the present case. A heat-activated adhesive layer 225 is provided on the side facing away from the carrier foil 210. By means of this heat-activated adhesive layer 225, the transfer sheet adheres to the base according to the structure predetermined by the stamping die, i.e. for example to the case shell 10 in the present case, which can be produced from a thermoplastic material, for example from acrylonitrile butadiene styrene plastics material.

After application, the layer 224 is located below the protective coating layer containing the phosphorescent particles. Here, a transparent material 224 and/or white coating can be provided in order to enhance the effect of the phosphorescence. The layer 224 can also be completely omitted.

The layer 222 can also be a decorative and/or functional layer. This layer 222 can also optionally be omitted.

The above-mentioned metal-coating can also be carried out indirectly in that a metal coating is provided in one or both of the layers 222 and 224. In this respect, the metal coating can also be applied by hot stamping in the above-mentioned region 60. Optionally, in the region 60, a layer can be applied by means of a separate hot stamping process using a further hot stamping foil having a comparable structure, by means of which a metal coating is provided in the layer 223, for example, with the phosphorescent particles being omitted from the further hot stamping foil. Two hot stamping processes are easier to implement than the one hot stamping process and additional steps of metal-coating involving etching, galvanizing, etc. It should be noted that, in general, a metal-coating layer can be applied by hot stamping even to first aid cases that do not have phosphorescent material applied by hot stamping.

Each of the layers 222 and 224 can also comprise a plurality of sub-layers.

The thickness ratios shown in FIG. 4 are not to scale.

The individual layers of the transfer sheet are between 1 nm and 50 μm thick.

FIG. 24 shows an emergency case according to the invention, denoted by 100 as a whole, which comprises two case shells 10 and 10′. For the sake of improved clarity, the seal 20, which an emergency case 100 according to the invention comprises, is not shown in FIG. 24 (cf. FIG. 24b for a corresponding view of the same emergency case 100 with the seal 20). The case shells 10 and 10′ are not identical in the embodiment shown here. The case shell 10′ that is located substantially in the upper half is formed to be flat and serves primarily as a lid. The lower case shell 10 comprises a distinctly pronounced indentation and serves to receive first aid materials and/or first aid devices, such as plasters and dressings, disposable gloves, first aid scissors and tweezers, hygienic face masks, hygienic clothing, emergency showers and eyewash kits, disinfectants and dispensers therefor, defibrillators and accessories, respiratory masks, and the like.

A peripheral receiving groove is arranged along the edge of the upper case shell 10′, which groove serves to receive the seal 20 (not shown in FIG. 24). It is arranged within the receiving groove 40 and is materially bonded to the case shell 10′. In the closed state of the emergency case 100 (cf. FIG. 24c for a corresponding view), both case shells 10 and 10′ are in contact with the seal 20. As a result, the interior of the emergency case 100 is sealed from the external environment, and so the risk of contamination of the contents of the emergency case 100 by germs or dirt particles getting in from the outside, for example, is reduced. In addition, the possibility of moisture getting in from the outside, which can compromise the safe operation of first aid materials or first aid devices, is thus minimized.

In order to increase the strength of the sealing, a pressing element 30 is arranged to run around the edge of the case shell 10, which pressing element exerts mechanical pressure on the seal 20 (not shown in FIG. 24) in the closed state of the emergency case 100. For this purpose, in the embodiment shown here, the pressing element 30 and the receiving groove 40 are formed and arranged relative to one another such that the pressing element 30 projects into the receiving groove 40 in the closed state of the emergency case 100. Unlike the exemplary embodiment shown here, it is likewise conceivable for the seal 20 to project beyond the receiving groove 40 and for the pressing element 30 to accordingly exert mechanical pressure on the seal 20 in the closed state of the emergency case 100 without the pressing element 30 projecting into the receiving groove 40. Owing to the deformation of the seal 20 that this brings about, the contact surface between the seal 20 and the emergency case half 10 is enlarged, meaning that the strength of the sealing of the interior of the emergency case 100 from the external environment is increased. This effect is achieved to a particularly great extent if the pressing element 30, as mentioned above, projects into the receiving groove 40 in the closed state of the emergency case 100.

An exemplary, particularly advantageous approach to producing the material bond of the sealing material to the case shell 10′, with an emergency case 100 according to the invention not being explicitly limited thereto and having been produced using this approach, consists in applying a flowable sealing component to the case shell 10′ using a machine, wherein the flowable sealing component forms a chemical bond with the surface of the case shell 10′ and itself reacts to form the sealing material in order to form a seal 20. This exemplary approach can be attributed to formed-in-place foam gasket technology (FIPFG).

FIG. 24b shows the same emergency case 100 as in FIG. 24, with the view in FIG. 24b showing the seal 20, which is arranged within the receiving groove 40 (not shown in FIG. 24b, since it is concealed by the seal 20) and is materially bonded to the case shell 10′.

It can be seen that the pressing element 30 exerts mechanical pressure on the seal 20 in the closed state of the emergency case 100 by projecting into the seal 20. As a result, in the closed state, the interior of the emergency case 100 is even more strongly sealed from the external environment than would be the case without the pressing element 30, since the seal 20 would only be in slight contact with the case shell 10.

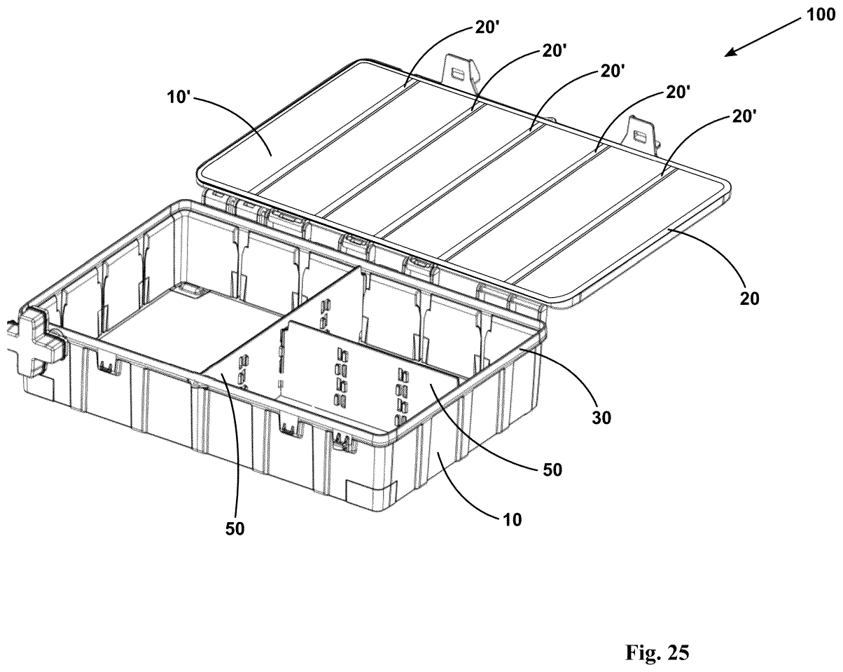

FIG. 24c, which shows the emergency case 100 from FIGS. 24 and 25 in the closed state, shows that the case shells 10 and 10′ have a substantially identical outline, wherein the case shells 10 and 10′ latch to one another by means of clamping tabs and latching lugs. By means of this latching, the mechanical pressure on the seal 20 (not shown in FIG. 24c) is maintained by the pressing element 30 of the case shell 10 and the interior of the emergency case 100 is reliably sealed from the external environment in the closed state.

FIG. 25 shows the option of variably forming sub-sections by means of partition elements 50 that can be inserted into the case shell 10. A plurality of seals 20′ are arranged on the case shell 10′ opposite the insertable partition elements 50, which seals are oriented transversely to the longitudinal axis of the emergency case 100 in this exemplary view. In this way, by appropriately arranging the insertable partition elements 50, a large number of sealed sub-sections can be produced. In this case, the seals 20′ contact the longitudinal edges of the insertable partition elements 50 oriented transversely to the longitudinal axis of the emergency case 100. The sub-sections are thus protected against contamination originating from the outside, such as germs or dirt particles. These sealed sub-sections are also protected against contamination originating from other sections of the emergency case 100. The emergency case 100 also comprises a pressing element 30 on the upper edge of the case shell 10 and a peripheral seal 20 on the case shell 10′. Similarly to the description of FIG. 24, the pressing element 30 accordingly exerts mechanical pressure on the seal 20 in the closed state of the emergency case 100, meaning that the strength of the sealing of the interior of the emergency case 100 from the external environment is increased. Alternatively or additionally to the exemplary embodiment described here, additional seals 20′ can be arranged on the case shell 10′ such that the longitudinal edges of those insertable partition elements 50 that are oriented in parallel with the longitudinal axis of the emergency case 100 are contacted by the seals 20′ once this case is closed.

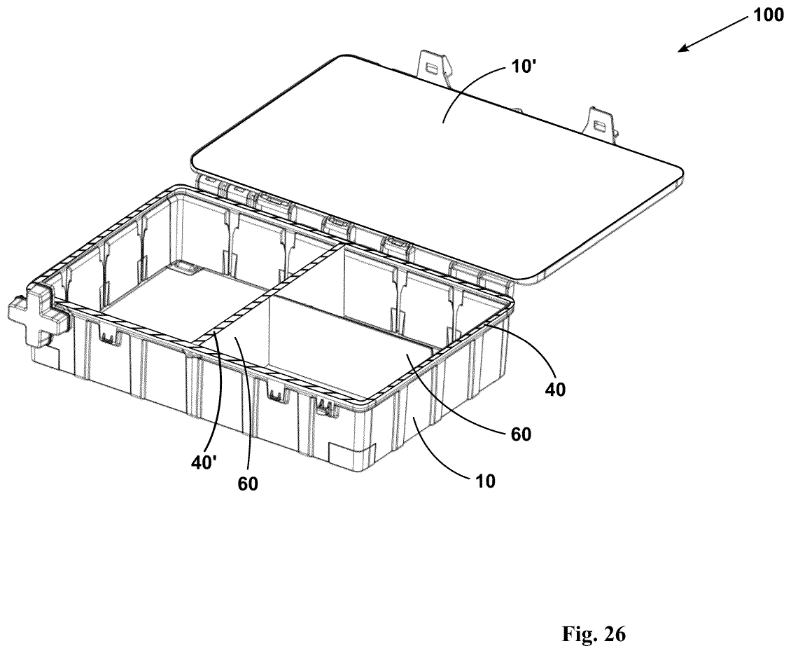

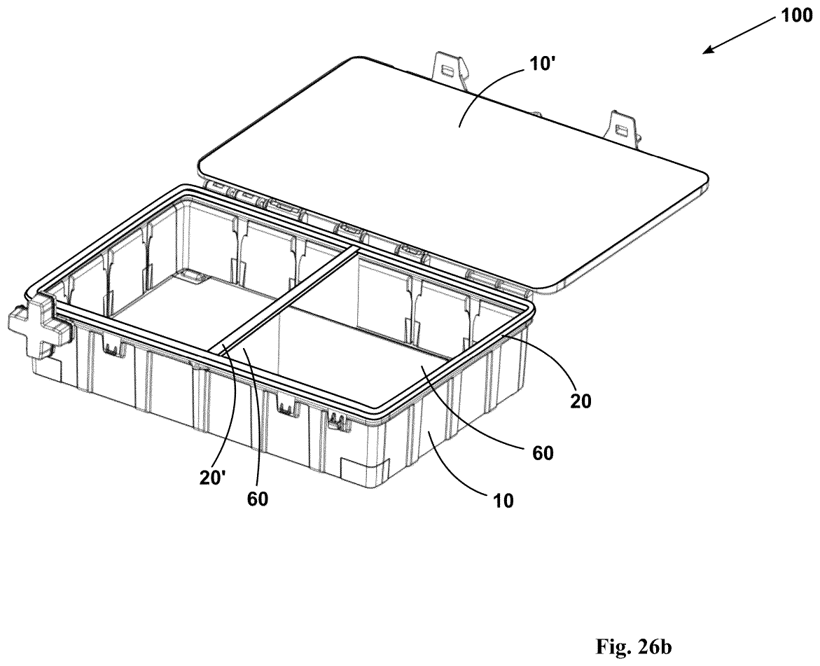

FIG. 26 shows a second embodiment of an emergency case according to the invention, denoted by 100 as a whole, in which fixed partition elements 60 having upwardly pointing longitudinal edges are formed on the case shell 10 for forming fixed sub-sections. For the sake of improved clarity, the seal 20, which an emergency case 100 according to the invention comprises, is not shown in FIG. 26 (cf. FIG. 26b for a corresponding view of the same emergency case 100 with the seal 20). The fixed sub-sections, which are formed by the fixed partition elements 60, serve to separately store first aid materials and/or first aid devices. In addition, the longitudinal edge of one of the fixed partition elements 60 comprises a receiving groove 40′ for receiving a seal 20′ (not shown in FIG. 26). Instead of a pressing element 30, in the embodiment shown here, the case shell 10 comprises a peripheral receiving groove 40 for receiving a seal 20 on the upper edge.

By contrast with the emergency case 100 from FIGS. 24 to 24c, the case shell 10′ shown here also does not comprise a receiving groove 40. The surface of the case shell 10′ pointing toward the interior of the emergency case 100 is instead substantially planar, in order to come into contact with the seals 20, 20′ (not shown in FIG. 26) arranged in the receiving grooves 40 and 40′ in the closed state of the emergency case 100. Alternatively or additionally, analogously to the case shell 10 of the emergency case 100 shown in FIG. 24-25b, the case shell 10′ could comprise one or more pressing elements 30, which project into the receiving grooves 40 and 40′ in the closed state of the emergency case 100. This situation clarifies that the arrangement of the receiving groove 40, 40′ or the pressing element 30 or a plurality of the two is explicitly not linked to a specific case shell 10 or 10′, but instead both elements can be arranged on corresponding case shells 10, 10′ so as to be opposite one another.

FIG. 26b shows the configuration of the embodiment of the emergency case 100 according to the invention, as shown in FIG. 26, with the seal 20 being shown in FIG. 26b. The seal 20 is arranged within the receiving groove 40 (not shown in FIG. 26b) on the upper edge of the case shell 10, is materially bonded to the case shell 10, and projects upward beyond the receiving groove 40. In an analogous manner, the seal 20′ is arranged within the receiving groove 40′ (not shown in FIG. 26b) on the longitudinal edge of one of the fixed partition elements 60, is materially bonded thereto, and projects beyond the receiving groove 40 in a manner analogous to the seal 20. By the seals 20, 20′ projecting upward beyond the receiving grooves 40, 40′, the planar surface of the case shell 10′ comes into contact with the seals 20, 20′ in the closed state of the emergency case 100 and also exerts mechanical pressure thereon, which increases the strength of the sealing of the interior of the emergency case 100 from the external environment. In this way, the additional seal 20′ generates a sub-section of the emergency case 100 that is sealed from the rest of the interior.

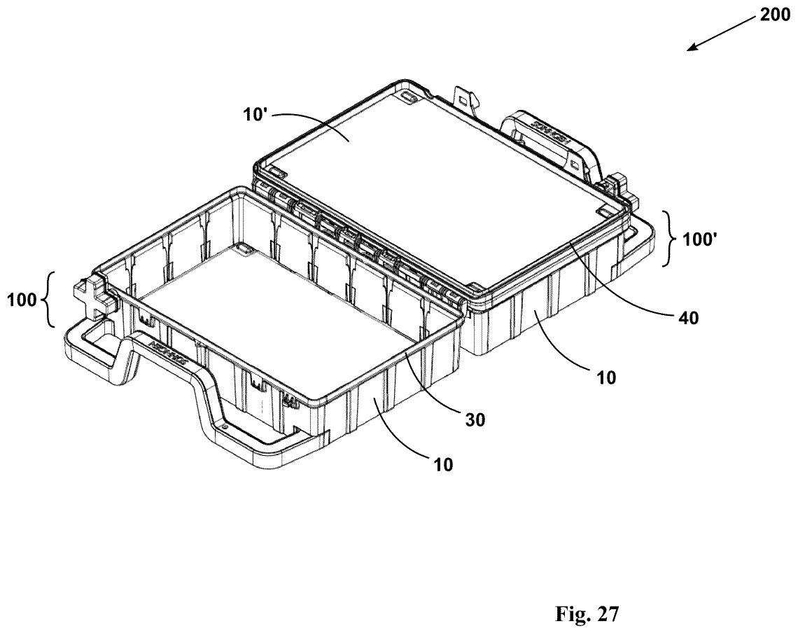

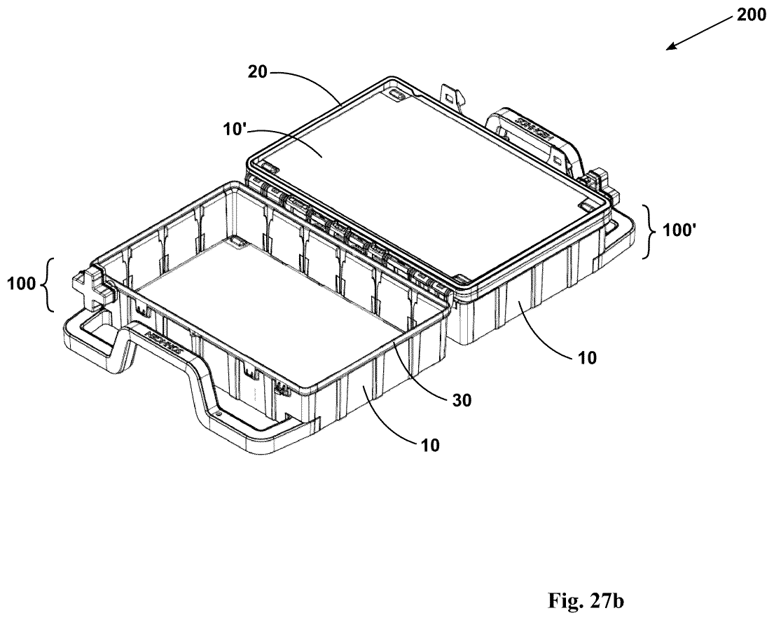

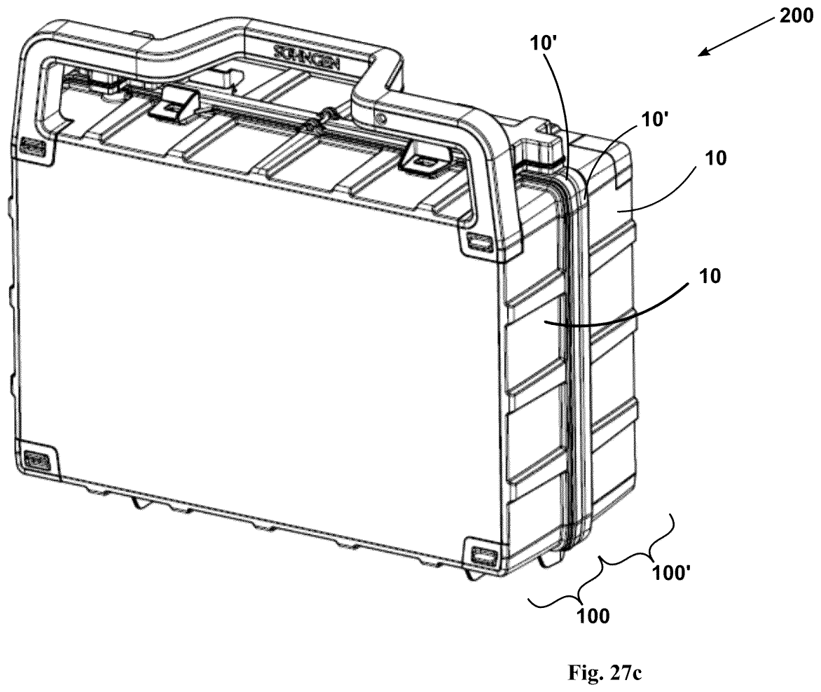

FIG. 27 shows a double emergency case according to the invention, denoted by 200 as a whole, which comprises two emergency cases 100 and 100′ according to the invention. In this case, the emergency cases 100, 100′ each have the same construction as the emergency case 100 shown in FIGS. 24 to 24c, with the seal 20 not being shown for the sake of improved clarity (cf. FIG. 27b for a corresponding view with the seal 20). When the emergency case 100 is open, which is substantially shown in the left half of FIG. 27, the pressing element 30 can be seen on the case shell 10 here.

The receiving groove 40 for receiving the seal 20 (not shown) is arranged on the case shell 10′. The emergency cases 100 and 100′ are detachably connected to one another by means of connection devices that are complementary to one another and are arranged on the upper edge of the side facing the respectively other case shell 100 or 100′. In this case, a case shell 10′ for forming a closed emergency case 100, 100′ can be locked to the corresponding case shell 10 of the same emergency case 100, 100′ in each case.

FIG. 27b shows the configuration of the embodiment of the double emergency case 200 according to the invention from FIG. 27, with the seal 20 being shown in FIG. 27b. The seal 20 is arranged within the receiving groove 40 (not shown in FIG. 27b) running around the edge of the case shell 10′ and is materially bonded to the case shell 10′. In the closed state, the seal 20 is in contact with the case shell 10, wherein the pressing element 30 exerts additional mechanical pressure on the seal 20, and so the strength of the sealing of the respective interiors of the emergency cases 100, 100′ from the external environment is increased. The same applies to the emergency case 100′, the interior construction of which is concealed in FIG. 27b by the opened-up case shell 10′.

FIG. 27c, which shows the double emergency case 200 from FIGS. 27 and 27b in the closed state, clarifies that the emergency cases 100, 100′ have substantially the same configuration. By locking the case shells 10′ to the respectively corresponding case shell 10 in order to produce a closed emergency case 100, 100′, it is ensured that the seals 20 are each in contact with the corresponding case shells 10, wherein a constant additional mechanical pressure acts on the seals 20 (not shown) by means of the pressing elements 30 (not shown), and so the strength of the sealing of the respective interiors of the emergency cases 100, 100′ from the external environment is increased.

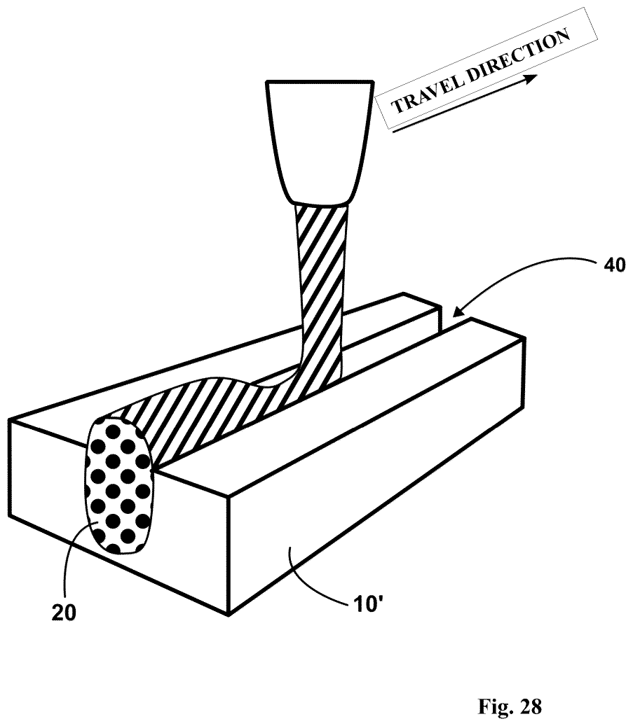

FIG. 28 is a snapshot of the application of the flowable sealing component, as can be carried out according to an embodiment of the method according to the invention. In this case, the application is carried out by means of an applicator, which is shown on the upper edge of FIG. 28. A sealing component flows out of said applicator into a receiving groove 40, which is arranged on the case shell 10′, while the applicator is moved along the receiving groove 40 in the direction indicated by the arrow labeled “movement direction” on the upper edge of FIG. 28. In this case, the flowable sealing component is preferably metered and guided along the receiving groove 40 using a machine. The flowable sealing component forms a chemical bond with the surface of the case shell and reacts, while foaming, to form a sealing material which forms a seal 20 while the applicator is moved further along the receiving groove 40, such that the sealing material does not come into contact with the applicator owing to the time delay of the foaming. In this exemplary view, the seal 20 projects beyond the receiving groove 40.

Although the invention has been described with reference to specific embodiments, it should go without saying that the invention is not limited to these examples and that numerous modifications can be intended and conceived by a person skilled in the art once they have considered the examples.

What is claimed is:

1. First-aid case (100), characterized in that at least one partial area of the first-aid case (100) contains a phosphorescent material.

2. First-aid case (100) according to claim 1, characterized in that the first-aid case (100) has two case shells (10) and the partial regions which contain the phosphorescent material are attachments which can be attached to the case shells (10).

3. First-aid case (100) according to claim 2, characterized in that the attachable add-on parts are selected from at least one of the following elements:
Handle (20),
component of the handle (20), in particular insert part (30),
fastener (40),
component of the fastener (40), in particular actuating element,
stack centering device (50),
decorative strip.

4. First-aid case (100) according to claim 2, characterized in that the handle (20) and/or a case shell (10) contains or contain phosphorescent material.

5. First-aid case (100) according to claim 1, characterized in that the phosphorescent material has an afterglow duration of at least 1 h, preferably at least 2 h, particularly preferably at least 3 h.

6. First-aid case (100) according to claim 1, characterized in that the mass fraction of the phosphorescent material in the partial regions or the case shell (10) is at least 10%, preferably at least 30%.

7. First-aid case (100) according to claim 1, characterized in that the phosphorescent material is a pigment containing particles of SrAl2O4:Eu, Dy, which preferably have mean diameters d50=25+4 μm and d90≤60 μm.

8. First-aid case (100) according to claim 1, characterized in that the first-aid case (100) is filled with at least one of:
plasters and dressing material,
disposable gloves,
first aid scissors and tweezers,
hygienic face mask,
hygienic clothing,
emergency showers and eyewash,
disinfectants and dispensers,
defibrillators and accessories,
respiratory masks.

9. First-aid case (100) according to claim 1, characterized in that at least partial surface regions (60) are metallized.

10. A method of manufacturing a first aid case (100) comprising
providing two case shells (10) and a handle (20);
providing add-on parts which can be attached to the case shells (10) and/or the handle (20) and which contain a phosphorescent material;
connecting the two case shells (10) to form a first aid case (100);
attaching the handle (20) to a case shell (10);
attaching the attachable attachments to the case shells (10) and/or the handle (20).

11. Method according to claim 10, wherein
the case shells (10) and the handle (20) are produced from a thermoplastic material, preferably from an acrylonitrile-butadiene-styrene plastic, in a first injection molding process;
the attachable attachment parts are produced in a second injection molding process from a thermoplastic material, preferably from an acrylonitrile-butadiene-styrene plastic, the phosphorescent material being added to the plastic prior to the injection molding process.

12. Method according to claim 11, wherein phosphorescent material is added to the thermoplastic for the production of the case shells (10) and/or the handle (20) before the first injection molding process.

13. A first aid case (100), characterized in that at least one section of the first aid case (100) contains a phosphorescent material, wherein the phosphorescent material has been applied to an outer surface of the first aid case (100) in one layer as part of a hot stamping process.

14. The first aid case (100) according to claim 13, characterized in that the phosphorescent material is provided in the form of a pigment which contains particles of SrAl₂O₄:Eu, Dy, which preferably have an average diameter d90≤5 μm.

15. The first aid case (100) according to claim 13, characterized in that the layer containing the phosphorescent material has a layer thickness of between 5 and 15 g/m², preferably of between 9 and 13 g/m², particularly preferably of between 10 and 12 g/m².

16. The first aid case (100) according to claim 13, characterized in that the phosphorescent material is provided in one layer, which also contains a protective coating and/or a colored coating.

17. The first aid case (100) according to claim 13, characterized by a further layer, also applied in the hot stamping process, between the layer containing the phosphorescent material and the outer surface, wherein the further layer is transparent or comprises a white coating.

18. The first aid case (100) according to claim 13, characterized in that the layer containing the phosphorescent material provides decoration and/or information, in particular in the form of lettering and/or at least one symbol.

19. The first aid case (100) according to claim 13, characterized in that the first aid case (100) comprises two case shells (10) and the outer surface is provided on an outer wall of one case shell (10).

20. The first aid case (100) according to claim 13, in which the first aid case (100) comprises two case shells (10) and bodies are add-on parts which can be affixed to the case shells (10) in the form of sections which contain the phosphorescent material.

21. The first aid case (100) according to claim 20, characterized in that the add-on parts that can be affixed are selected at least from one of the following elements:
handle (20),
component of the handle (20), in particular insert part (30),
closure (40),
component of the closure (40), in particular actuating element,
stacking centering apparatus (50),
decorative strip.

22. The first aid case (100) according to claim 20, characterized in that the handle (20) and/or a case shell (10), in the form of bodies, contain phosphorescent material.

23. The first aid case (100) according to claim 13, characterized in that the phosphorescent material has a time of persistence of at least 1 hour, preferably at least 2 hours, particularly preferably at least 3 hours.

24. The first aid case (100) according to claim 13, characterized in that at least one body of the first aid case (100) consists of a material that contains phosphorescent material, wherein the mass percentage of the phosphorescent material in the body is at least 10%, preferably at least 30%.

25. The first aid case (100) according to claim 13, characterized in that the first aid case (100) is filled with at least one of:
plasters and dressings,
disposable gloves,
first aid scissors and tweezers,
hygienic face masks,
hygienic clothing,
emergency showers and eyewash kits,
disinfectants and dispensers therefor,
defibrillators and accessories,
respiratory masks.

26. The first aid case (100) according to claim 13, characterized in that at least one surface section (60) is metal-coated.

27. A method for producing a first aid case (100), which comprises:
providing two case shells (10) and a handle (20);
performing at least one hot stamping process, wherein, in the entirety of all the hot stamping processes, a layer containing phosphorescent particles is applied to at least one of the case shells (10) and/or the handle (20);
connecting the two case shells (10) to form a first aid case (100);
affixing the handle (20) to a case shell (10).

28. The method according to claim 27, characterized in that each hot stamping process is performed using a hot stamping die, which is brought to a temperature of between 130° C. and 165° C. and/or is pressed against the case shell (10) or the handle (20) for a duration of between 0.25 seconds and 5 seconds and/or is pressed against the case shell (10) or the handle (20) at a pressure of between 3 and 9 bar, preferably between 5 and 7 bar and/or is pressed against the case shell (10) or the handle (20) by applying a weight of between 600 and 1000 kg, preferably between 720 and 880 kg.

29. The method according to claim 27, characterized in that
the case shells (10) and the handle (20) are produced from a thermoplastic material, preferably from an acrylonitrile butadiene styrene plastics material, in an injection-molding process.

30. The method according to claim 29, wherein phosphorescent material is added to the thermoplastic material for producing the case shells (10) and/or the handle (20) before the injection-molding process.

31. The method according to claim 27, characterized in that, in each hot stamping process, a hot stamping foil is used, which comprises a transfer sheet that contains phosphorescent particles of SrAl₂O₄:Eu, Dy.

32. The method according to claim 31, characterized in that the phosphorescent particles have average diameters d₉₀≤5 μm.

33. The method according to claim 27, comprising the additional step of:
metal-coating at least surface sections (60) of the case shells (10) and/or the handle (20), wherein the metal-coating is preferably implemented by means of the following steps:
etching the surface sections (60) of the first aid case (100) with a solution consisting of chromic acid, sulfuric acid, and water, wherein the etching is carried out at a temperature of at least 60° C., preferably at 70° C.;
activating the surface sections (60) with palladium, wherein the palladium is colloidally dissolved and is surrounded by a protective colloid shell or is present in ionic form;
wetting the surface sections (60) with an accelerator solution, wherein the accelerator solution is selected such that it reduces ionically present palladium or, in the case of colloidally dissolved palladium, removes the protective colloid shell surrounding the colloidal palladium;
chemically depositing nickel on the surface sections (60);
galvanically depositing copper on the nickel-plated surface sections (60);
galvanically depositing nickel on the copper-plated surface sections (60);
galvanically depositing chromium on the surface sections (60) nickel-plated by galvanic deposition;
drying the surface sections (60), preferably at a temperature of 67° C.,
wherein the surface sections (60) are rinsed with water, preferably with deionized water, between the individual steps.
